(19) United States
(12) Patent Application Publication
(10) Pub. No.: US 2021/0189444 A1
(43) Pub. Date: Jun. 24, 2021

(54) METHOD OF PURIFYING CANNABINOIDS FROM YEAST FERMENTATION BROTH

(71) Applicant: Demetrix, Inc., Berkeley, CA (US)

(72) Inventors: Catherine ALVIAR, Berkeley, CA (US); Martinus Johannes Arnoldus DAAS, Berkeley, CA (US)

(21) Appl. No.: 17/127,570

(22) Filed: Dec. 18, 2020

Related U.S. Application Data
(60) Provisional application No. 62/951,877, filed on Dec. 20, 2019.

(51) Int. Cl. C12P 17/06; C12P 7/42; C12N 1/16; B01D 11/02

(52) U.S. Cl. CPC C12P 17/06; B01D 11/02; C12N 1/16; C12P 7/42

(57) ABSTRACT

Provided herein are methods of purifying cannabinoids or cannabinoid derivatives produced using modified host cells and recovering the resulting cannabinoid or cannabinoid derivative preparations. The present methods provide preparations comprising cannabinoids or cannabinoid derivatives having increased purity and amounts of the cannabinoids or cannabinoid derivatives.

### CROSS-REFERENCE TO RELATED APPLICATIONS

This application claims the benefit of U.S. Provisional Application No. 62/951,877, filed Dec. 20, 2019, the contents of which are incorporated herein by reference in its entirety.

### INTRODUCTION

Plants from the genus Cannabis have been used by humans for their medicinal properties for thousands of years. In modern times, the bioactive effects of Cannabis are attributed to a class of compounds termed “cannabinoids,” of which there are hundreds of structural analogs including tetrahydrocannabinol (THC) and cannabidiol (CBD). These molecules and preparations of Cannabis material have recently found application as therapeutics for chronic pain, multiple sclerosis, cancer-associated nausea and vomiting, weight loss, appetite loss, spasticity, seizures, and other conditions.

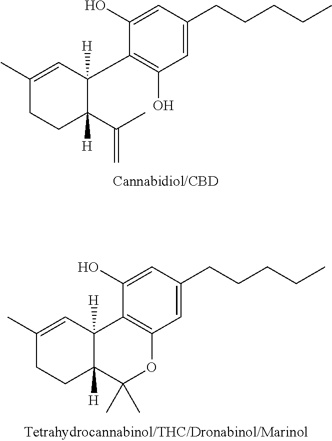

The physiological effects of certain cannabinoids are thought to be mediated by their interaction with two cellular receptors found in humans and other animals. Cannabinoid receptor type 1 (CB1) is common in the brain, the reproductive system, and the eye. Cannabinoid receptor type 2 (CB2) is common in the immune system and mediates therapeutic effects related to inflammation in animal models. The discovery of cannabinoid receptors and their interactions with plant-derived cannabinoids predated the identification of endogenous ligands.

Besides THC and CBD, hundreds of other cannabinoids have been identified in Cannabis. However, many of these compounds exist at low levels and alongside more abundant cannabinoids, making it difficult to obtain pure samples from plants to study their therapeutic potential. Similarly, methods of chemically synthesizing these types of products have been cumbersome and costly, and tend to produce insufficient yield. Accordingly, additional methods of making pure cannabinoids or cannabinoid derivatives (e.g., acidic cannabinoids, acidic cannabinoid derivatives, neutral cannabinoids, and neutral cannabinoid derivatives) are needed.

One possible method is production via fermentation of engineered microbes, such as yeast. By engineering production of the relevant plant enzymes in microbes, it may be possible to achieve conversion of various feedstocks into a range of cannabinoids and cannabinoid derivatives (e.g., acidic cannabinoids, acidic cannabinoid derivatives, neutral cannabinoids, and neutral cannabinoid derivatives) potentially at much lower cost and with much higher purity than what is available from the plant.

### SUMMARY

While cannabinoids are naturally produced by Cannabis plants, using plants for the production of cannabinoids and/or cannabinoid derivatives that will be recovered and purified is challenging. Because the cannabinoids and/or cannabinoid derivatives are retained in the plants (e.g. in the trichome), recovery of a cannabinoid and/or cannabinoid derivative of interest requires breaking down the cells either through, for example, mechanical or chemical means. This releases unwanted plant cell components and contaminants (e.g., chlorophyll, lipids, sterols, fatty acids, salts, sugars, and heavy waxes) into the cannabinoid and/or cannabinoid derivative preparation. Additionally, the removal of these plant cell components and contaminants requires additional steps that themselves may introduce unwanted contaminants (e.g., ethanol, butane, propane, toxic solvents, and/or supercritical carbon dioxide). Recovery of cannabinoids and/or cannabinoid derivatives also requires separating the cannabinoid and/or cannabinoid derivative from these contaminants (e.g., through winterization and filtration, distillation, and/or column chromatography). These various required purification steps increases the time required and the cost for recovery of the cannabinoid and/or cannabinoid derivative, while also reducing overall cannabinoid and/or cannabinoid derivative yield. These problems are solved by the present disclosure.

Extraction of cannabinoids from the Cannabis plant presents a number of challenges, especially if highly pure fractions or single cannabinoid isolates are desired. Mechanical extraction methods such as cold water extraction to make hash, or grinding and sieving to isolate kief can be employed to separate trichomes from the rest of the plant tissue, yielding a crude fraction containing a mixture of cannabinoids, plus terpenes and other plant components. Higher purity can be obtained using solvent extractions, where the input is plant, whole or ground, in the dried or fresh state. Ethanol extraction, e.g. with Soxhlet extractor, purifies cannabinoids and terpenes, but chlorophyll and wax contamination are typical. While there are no health concerns associated with residual ethanol, the use of ethanol at large scale in this process presents a fire hazard. Butane and propane are also used to extract cannabinoids from plant material. Butane hash oil is typically 75% cannabinoid, 2-7% lighter components such as terpenes, solvents and water, and the remaining 18-43% heavier components such as lipids, sterols, chlorophyll, fatty acids, salts, sugars and heavy waxes. Residual solvent is toxic, and must be completely removed before consumer use, and the solvents are a fire hazard. Supercritical CO₂ extraction provides more control over the profile of solubilized compounds, is non-toxic, and does not present a fire hazard. However, a large amount of waxes and fatty acids are typically co-purified. All of the above solvent methods listed yield a mixture of cannabinoids with significant plant matter contamination. To achieve more purified fractions or single cannabinoid isolates, additional purification steps are required. Winterization and filtration can be employed to remove waxes and fatty acids. Distillation can separate the cannabinoid fraction from terpenes and other components yielding 85-95% purity. To purify single cannabinoids, column or other chromatography methods are utilized, but cannabinoids with very similar polarity or hydrophobicity are difficult or impractical to separate commercially.

The present disclosure provides methods of preparing cannabinoid or cannabinoid derivative preparations comprising cannabinoids and/or cannabinoid derivatives (e.g., acidic cannabinoids and/or acidic cannabinoid derivatives or neutral cannabinoids and/or neutral cannabinoid derivatives) produced using modified host cells, such as those disclosed in WO2018/200888 filed Apr. 27, 2018 and WO 2020/069214 filed Sep. 26, 2019, and PCT/US2020/051261 filed Sep. 17, 2020. These methods include a streamlined process that does not require preparatory high-performance liquid chromatography (HPLC). Moreover, the methods disclosed herein do not require steps to lyse or break-down cells, because cannabinoids and/or cannabinoid derivatives (e.g., acidic cannabinoids, acidic cannabinoid derivatives, neutral cannabinoids, and neutral cannabinoid derivatives) are secreted and/or passively diffuse from modified host cells or are associated with the cell wall and/or cell membrane, enabling recovery from the fermentation broth (including whole fermentation broth, fermentation liquid, cell culture (e.g., whole cell culture or whole cell broth), and yeast fermentation broth). Eliminating the need for cell lysis or break-down eases separation of desired cannabinoids and cannabinoid derivatives and the generation of the desired cannabinoids and cannabinoid derivative preparations, as the amount of cell debris is limited and the contents of the cells are not released. In the presence of heat or light, acidic cannabinoids or cannabinoid derivatives may be decarboxylated to afford neutral cannabinoids or neutral cannabinoid derivatives.

The methods of the present disclosure provide cannabinoid or cannabinoid derivative preparations comprising substantially pure cannabinoids or cannabinoid derivatives (e.g., acidic cannabinoids, acidic cannabinoid derivatives, neutral cannabinoids, and neutral cannabinoid derivatives).

In some aspects, the present disclosure provides methods of recovering cannabinoid or cannabinoid derivative preparations comprising cannabinoids or cannabinoid derivatives (e.g., acidic cannabinoids, acidic cannabinoid derivatives, neutral cannabinoids, and neutral cannabinoid derivatives) produced using modified host cells, such as those disclosed in WO2018/200888 filed Apr. 27, 2018 and WO 2020/069214 filed Sep. 26, 2019, and PCT/US2020/051261 filed Sep. 17, 2020.

The disclosure provides cannabinoid or cannabinoid derivative preparations comprising substantially pure cannabinoids or cannabinoid derivatives (e.g., acidic cannabinoids, acidic cannabinoid derivatives, neutral cannabinoids, and neutral cannabinoid derivatives).

Further, the present disclosure provides formulations, including GRAS formulations and pharmaceutical formulations, as well as methods, uses, cannabinoid and cannabinoid derivative preparations for use, medicaments, and formulations (including GRAS formulations and pharmaceutical formulations) for use comprising said cannabinoid and cannabinoid derivative preparations.

In some aspects, the present disclosure provides a cannabinoid or cannabinoid derivative preparation prepared from a fermentation broth, the cannabinoid or cannabinoid derivative preparation comprising at least 85 weight % of a cannabinoid or cannabinoid derivative. In some embodiments, the cannabinoid or cannabinoid derivative preparation comprises at least 90 weight % of a cannabinoid or cannabinoid derivative. In some embodiments, the cannabinoid or cannabinoid derivative preparation comprises at least 95 weight % of a cannabinoid or cannabinoid derivative.

In some embodiments, the cannabinoid or cannabinoid derivative preparation is substantially free of impurities. In some embodiments, the cannabinoid or cannabinoid derivative preparation is substantially free of one or more of pentyldiacetic acid lactone (PDAL), hexanoyl triacetic acid lactone (HTAL), olivetol, olivetolic acid, and hexanoic acid. In some embodiments, the cannabinoid or cannabinoid derivative preparation contains less than 5 weight % total of the combination of one or more of pentyldiacetic acid lactone (PDAL), hexanoyl triacetic acid lactone (HTAL), olivetol, olivetolic acid, and hexanoic acid. In some embodiments, the cannabinoid is a neutral cannabinoid, wherein the neutral cannabinoid is tetrahydrocannabinol (THC), cannabidiol (CBD), or cannabigerol (CBG). In some embodiments, the cannabinoid is an acidic cannabinoid, wherein the acidic cannabinoid is tetrahydrocannabinolic acid (THCA), cannabidiolic acid (CBDA), or cannabigerolic acid (CBGA).

In some embodiments, the cannabinoid or cannabinoid derivative preparation is a white crystalline solid at about 25° C.

In some embodiments, the fermentation broth comprises yeast cells, a culture medium, or both yeast cells and culture medium.

In some aspects, the present disclosure provides a method of preparing a cannabinoid or cannabinoid derivative preparation, said method comprising the steps of: 1) extracting an acidic cannabinoid or acidic cannabinoid derivative from a fermentation broth into an emollient phase; 2) extracting the acidic cannabinoid or acidic cannabinoid derivative in emollient into an aqueous phase; 3) decarboxylating the acidic cannabinoid or acidic cannabinoid derivative in the aqueous phase to afford a neutral cannabinoid or neutral cannabinoid derivative; 4) crystallizing the neutral cannabinoid or neutral cannabinoid derivative; and 5) recovering the resulting cannabinoid or cannabinoid derivative preparation, wherein the cannabinoid or cannabinoid derivative preparation comprises at least 85 weight % of the neutral cannabinoid or neutral cannabinoid derivative. In some embodiments, the method comprises a step of washing the fermentation broth before extracting the acidic cannabinoid or acidic cannabinoid derivative into the emollient phase. In some embodiments, the method comprises a step of solubilizing the crystallized neutral cannabinoid or neutral cannabinoid derivative and recrystallizing the neutral cannabinoid or neutral cannabinoid derivative.

In some aspects, the present disclosure provides a method of preparing a cannabinoid or cannabinoid derivative preparation, said method comprising: extracting an acidic cannabinoid or acidic cannabinoid derivative from a fermentation broth using an emollient phase and recovering the resulting cannabinoid or cannabinoid derivative preparation, wherein the cannabinoid or cannabinoid derivative preparation comprises at least 85 weight % of a cannabinoid or cannabinoid derivative, wherein the cannabinoid or cannabinoid derivative is a neutral cannabinoid, a neutral cannabinoid derivative, the acidic cannabinoid, or the acidic cannabinoid derivative.

In some embodiments, the pH of the fermentation broth is between about 4.0 and about 10.0. In some embodiments, the pH of the fermentation broth is between about 5.0 and about 9.0. In some embodiments, the pH of the fermentation broth is between about 7.0 and about 8.0. In some embodiments, the pH of the fermentation broth is about 7.67.

In some embodiments, the extraction of the acidic cannabinoid or acidic cannabinoid derivative from the fermentation broth into the emollient phase is performed at a temperature between about 20° C. and about 50° C. In some embodiments, the extraction of the acidic cannabinoid or acidic cannabinoid derivative from the fermentation broth into the emollient phase is performed at a temperature between about 30° C. and about 50° C. In some embodiments, the extraction of the acidic cannabinoid or acidic cannabinoid derivative from the fermentation broth into the emollient phase is performed at a temperature of about 30° C.

In some embodiments, the extraction of the acidic cannabinoid or acidic cannabinoid derivative from the fermentation broth into the emollient phase is performed using about 10% v/v to about 20% v/v emollient. In some embodiments, at least about 50% of the acidic cannabinoid or acidic cannabinoid derivative present in the fermentation broth is extracted into the emollient phase. In some embodiments, at least about 90% of the acidic cannabinoid or acidic cannabinoid derivative present in the fermentation broth is extracted into the emollient phase. In some embodiments, at least about 95% of the acidic cannabinoid or acidic cannabinoid derivative present in the fermentation broth is extracted into the emollient phase. In some embodiments, one or more impurities present in the fermentation broth are not significantly extracted into the emollient phase with the acidic cannabinoid or acidic cannabinoid derivative. In some embodiments, the one or more impurities comprise olivetolic acid, PDAL, or HTAL, or a combination of any of the foregoing.

In some embodiments, the method comprises a step of extracting the acidic cannabinoid or acidic cannabinoid derivative in an emollient phase into an aqueous phase. In some embodiments, the extraction of the acidic cannabinoid or acidic cannabinoid derivative in the emollient phase into the aqueous phase is performed at a ratio of water:emollient phase between about 1:1 and about 5:1. In some embodiments, the extraction of the acidic cannabinoid or acidic cannabinoid derivative in the emollient phase into the aqueous phase is performed at a ratio of water:emollient phase of about 4:1. In some embodiments, the acidic cannabinoid or acidic cannabinoid derivative in the emollient phase is extracted into the aqueous phase at a pH of about 11.5.

In some embodiments, the extraction of the acidic cannabinoid or acidic cannabinoid derivative in the emollient phase into the aqueous phase is performed by: a) mild agitation; or b) vigorous mixing to hydrolyze the emollient phase.

In some embodiments, at least about 50% of the acidic cannabinoid or acidic cannabinoid derivative present in the emollient phase is extracted into the aqueous phase. In some embodiments, at least 90% of the acidic cannabinoid or acidic cannabinoid derivative present in the emollient phase is extracted into the aqueous phase. In some embodiments, the emollient phase comprises an oil, solvent, toluene, methyl isobutyl ketone (MIBK), heptanes, ethanol, methanol, isopropanol, isopropyl myristate (IPM), or any combination thereof. In some embodiments, the emollient phase comprises IPM.

In some embodiments, the cannabinoid or cannabinoid derivative is the neutral cannabinoid or neutral cannabinoid derivative.

In some embodiments, the method comprises a step of decarboxylating the acidic cannabinoid or acidic cannabinoid derivative to afford the neutral cannabinoid or neutral cannabinoid derivative. In some embodiments, decarboxylation of the acidic cannabinoid or acidic cannabinoid derivative is performed at temperatures greater than about 70° C. In some embodiments, decarboxylation of the acidic cannabinoid or acidic cannabinoid derivative is performed at a temperature between about 80° C. to about 140° C. In some embodiments, decarboxylation of the acidic cannabinoid or acidic cannabinoid derivative is performed at a temperature between about 90° C. to about 130° C. In some embodiments, decarboxylation of the acidic cannabinoid or acidic cannabinoid derivative is performed for about 5 minutes to about 20 hours. In some embodiments, decarboxylation of the acidic cannabinoid or acidic cannabinoid derivative is performed for about 14 hours. In some embodiments, decarboxylation of the acidic cannabinoid or acidic cannabinoid derivative is performed for about 20 minutes.

In some embodiments, the yield of the neutral cannabinoid or neutral cannabinoid derivative afforded by decarboxylation of the acidic cannabinoid or acidic cannabinoid derivative is at least about 50%. In some embodiments, the yield of the neutral cannabinoid or neutral cannabinoid derivative afforded by decarboxylation of the acidic cannabinoid or acidic cannabinoid derivative is at least about 60%. In some embodiments, the yield of the neutral cannabinoid or neutral cannabinoid derivative afforded by decarboxylation of the acidic cannabinoid or acidic cannabinoid derivative is between about 60% and about 100%.

In some embodiments, the method comprises a step of crystallizing the neutral cannabinoid or neutral cannabinoid derivative. In some embodiments, crystallization of the neutral cannabinoid or neutral cannabinoid derivative is performed by slowly cooling an aqueous phase comprising the neutral cannabinoid or neutral cannabinoid derivative afforded by decarboxylation of the acidic cannabinoid or acidic cannabinoid derivative to about 25° C. or below. In some embodiments, crystallization of the neutral cannabinoid or neutral cannabinoid derivative occurs at a pH of about 1.0 to about 10.0. In some embodiments, crystallization of the neutral cannabinoid or neutral cannabinoid derivative occurs at a pH of below about 12. In some embodiments, crystallization of the neutral cannabinoid or neutral cannabinoid derivative occurs at a pH of below about 2. In some embodiments, the yield of the cannabinoid or cannabinoid derivative preparation recovered after crystallization of the neutral cannabinoid or neutral cannabinoid derivative is at least 50%. In some embodiments, the yield of the cannabinoid or cannabinoid derivative preparation recovered after crystallization of the neutral cannabinoid or neutral cannabinoid derivative is at least 95%. In some embodiments, the neutral cannabinoid is tetrahydrocannabinol (THC), cannabidiol (CBD), or cannabigerol (CBG).

In some embodiments, the cannabinoid or cannabinoid derivative is the acidic cannabinoid or acidic cannabinoid derivative. In some embodiments, the method comprises a step of crystallizing the acidic cannabinoid or acidic cannabinoid derivative. In some embodiments, the yield of the cannabinoid or cannabinoid derivative preparation recovered after crystallization of the acidic cannabinoid or acidic cannabinoid derivative is at least 50%. In some embodiments, the yield of the cannabinoid or cannabinoid derivative is at least 95%. In some embodiments, the acidic cannabinoid is tetrahydrocannabinolic acid (THCA), cannabidiolic acid (CBDA), or cannabigerolic acid (CBGA).

In some embodiments, the fermentation broth comprises yeast cells, a culture medium, or both yeast cells and a culture medium.

In some aspects, the present disclosure provides a cannabinoid or cannabinoid derivative preparation preparable by the methods disclosed herein.

In some embodiments, the present disclosure provides a cannabinoid or cannabinoid derivative preparation prepared by the methods disclosed herein.

### BRIEF DESCRIPTION OF THE DRAWINGS

FIG. 1 shows an exemplary system comprising the downstream processing and purification steps disclosed herein. These steps may be performed in any appropriate order. One of more of these steps may be omitted or repeated (for example twice, three times, etc.)

FIG. 2 shows the amounts of cannabigerolic acid (CBGA), hexanoyl triacetic acid lactone (HTAL), olivetolic acid, pentyl diacetic acid lactone (PDAL), and olivetol washed out of the fermentation broth at pH 5.0 and pH 7.67. At pH 7.67, 10% of the CBGA, 85% of the HTAL and olivetolic acid, 75% of PDAL, and 25% olivetol was lost.

FIG. 3 shows the amounts CBGA and olivetolic acid extracted using isopropyl myristate (IPM) overlay at pH 5, pH 6.33, pH 7.67, and pH 9. At pH 7.67, 95% of the CBGA and olivetol was extracted, while the levels of olivetolic acid, PDAL, and HTAL extracted are below the limit of quantification (LOQ).

FIG. 4 shows an exemplary decarboxylation profile for CBGA and solubility curves for CBGA and cannabigerol (CBG). The difference in solubility of the two compounds provides the basis of their purification in the downstream process disclosed herein.

FIGS. 5A-B show decarboxylation of CBGA to CBG. Decarboxylation occurred in aqueous phase in closed vials at different temperatures (60° C. to 130° C. increased in 10° C. increments) for 0-24 hours.

FIG. 6 shows decarboxylation at 90° C. over time.

FIG. 7 shows the extraction of CBGA, olivetol, and olivetolic acid from IPM into an aqueous phase at pH 11.5. The pH of the aqueous phase was adjusted by either 25% KOH or 25% NaOH, which have similar extraction yields. However, under identical mixing conditions, a fluffy interface layer is seen using the 25% NaOH condition which hinders clean phase separation.

FIG. 8 shows CBGA extraction from IPM into a fresh aqueous layer: Optimizing IPM to aqueous layer ratio. Ratios of IPM to water of 1:10, 1:3, and 1:1 at pH 11.5 were tested. Most of the olivetol remains in the IPM after extraction, while olivetolic acid follows CBGA into the aqueous layer. The data in the examples disclosed herein were obtained using a 1:4 IPM to water ratio.

FIG. 9 shows CBGA extraction from IPM into a fresh aqueous layer: IPM wash before extraction. Two washes were performed at pH 7.67 (each had duration of 1 hour). The final extraction was performed at pH 11.5. The first two washes did not get rid of any impurities and/or byproducts, and the extraction of CBGA is maximized after about 20 minutes. As this step did not remove a significant amount of impurities and/or byproducts, it is an optional step in the downstream processing methods disclosed herein.

FIG. 10 shows precipitation of CBGA from aqueous layer via acidification. To precipitate CBGA, the pH of the solution is adjusted to below 2.0. The impurities and/or byproducts also precipitate out of solution, resulting in a brown sticky residue. This is an optional step, and the downstream processing methods disclosed herein may be simplified by proceeding to decarboxylation instead of acidifying the aqueous solution.

### DETAILED DESCRIPTION

Synthetic biology allows for the engineering of industrial host organisms—e.g., microbes—to convert simple sugar feedstocks into medicines and target molecules of interest. This approach includes identifying genes that produce the target molecules and optimizing their activities in the industrial host. Microbial production can be significantly cost-advantaged over agriculture and chemical synthesis, less variable, and allow tailoring of the target molecule. The present disclosure provides methods of preparing preparations comprising cannabinoids or cannabinoid derivatives produced using modified host cells, such as those disclosed in WO2018/200888 filed Apr. 27, 2018 and WO 2020/069214 filed Sep. 26, 2019, and PCT/US2020/051261 filed Sep. 17, 2020.

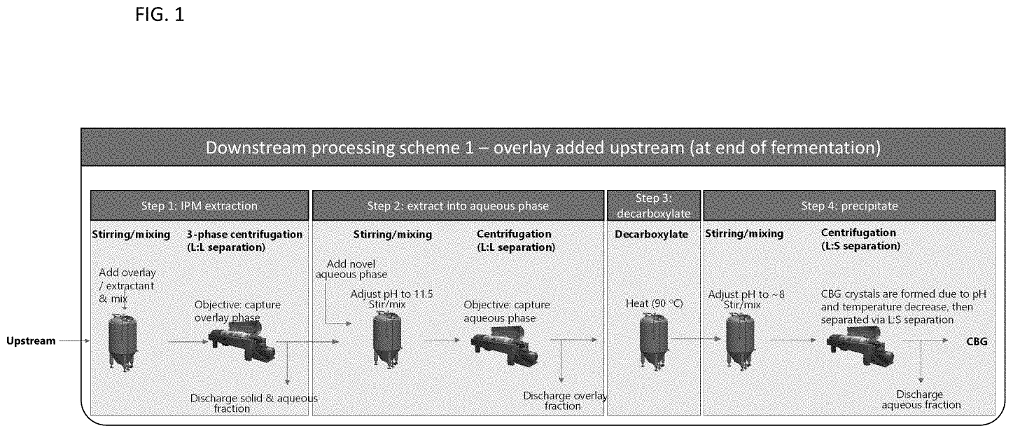

Disclosed herein are methods of preparing a preparation comprising cannabinoids or a cannabinoid derivatives produced using a modified host cell. The cannabinoid may be a neutral or an acidic cannabinoid and the cannabinoid derivative may be a neutral or an acidic cannabinoid derivative. Importantly, these methods do not require the use of preparative HPLC or other forms of chromatography. The methods disclosed herein may include the steps as depicted in FIG. 1 in any combination or order: 1) modified host cells in fermentation broth are washed to solubilize and separate impurities from the fermentation broth; 2) the fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) optionally, if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative is precipitated from an aqueous layer; and/or 7) the cannabinoid or cannabinoid derivative is crystallized. These steps may be performed in any combination or order. One or more of these steps may be omitted (e.g. decarboxylation of the acidic cannabinoid or the acidic cannabinoid derivative) or performed multiple times (e.g., twice, three times). In some embodiments, the methods disclosed herein do not include the steps of washing the modified host cells in fermentation broth and/or precipitating the cannabinoid or cannabinoid derivative. In some aspects of the present disclosure, acidic cannabinoids are crystallized in step 7, and thus do not require decarboxylation (e.g. step 5).

Notably, the methods of preparing a preparation comprising cannabinoids or cannabinoid derivatives of the present disclosure provide cannabinoid or cannabinoid derivative preparations comprising substantially pure cannabinoids or cannabinoid derivatives (e.g., acidic cannabinoids, acidic cannabinoid derivatives, neutral cannabinoids, and neutral cannabinoid derivatives).

The disclosure also provides cannabinoid or cannabinoid derivative preparations comprising substantially pure cannabinoids or cannabinoid derivatives (e.g., acidic cannabinoids, acidic cannabinoid derivatives, neutral cannabinoids, and neutral cannabinoid derivatives). Further, the present disclosure provides formulations, including GRAS formulations and pharmaceutical formulations, as well as methods, uses, cannabinoid and cannabinoid derivative preparations for use, medicaments, and formulations (including GRAS formulations and pharmaceutical formulations) for use comprising said cannabinoid or cannabinoid derivative preparations.

### General Information

In certain aspects, the practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature: “Molecular Cloning: A Laboratory Manual,” second edition (Sambrook et al., 1989); “Oligonucleotide Synthesis” (M. J. Gait, ed., 1984); “Animal Cell Culture” (R. I. Freshney, ed., 1987); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., eds., 1994). Singleton et al., Dictionary of Microbiology and Molecular Biology 2nd ed., J. Wiley & Sons (New York, N.Y. 1994), and March, Advanced Organic Chemistry Reactions, Mechanisms and Structure 4th ed., John Wiley & Sons (New York, N.Y. 1992), provide one skilled in the art with a general guide to many of the terms used in the present application. “Cannabinoid” or “cannabinoid compound” as used herein may refer to a member of a class of unique meroterpenoids found until now only in Cannabis sativa. Cannabinoids may include, but are not limited to, cannabichromene (CBC) type (e.g., cannabichromenic acid), cannabigerol (CBG) type (e.g., cannabigerolic acid), cannabidiol (CBD) type (e.g., cannabidiolic acid), Δ⁹-trans-tetrahydrocannabinol (Δ⁹-THC) type (e.g., Δ⁹-tetrahydrocannabinolic acid), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol (CBT) type, cannabigerolic acid (CBGA), cannabigerolic acid monomethylether (CBGAM), cannabigerol (CBG), cannabigerol monomethylether (CBGM), cannabigerovarinic acid (CBGVA), cannabigerovarin (CBGV), cannabichromenic acid (CBCA), cannabichromene (CBC), cannabichromevarinic acid (CBCVA), cannabichromevarin (CBCV), cannabidiolic acid (CBDA), cannabidiol (CBD), cannabidiol monomethylether (CBDM), cannabidiol-C₄ (CBD-C₄), cannabidiolic acid-C₄ (CBDA-C₄), cannabidivarinic acid (CBDVA), cannabidivarin (CBDV), cannabidiorcol (CBD-C₁), Δ⁹-tetrahydrocannabinolic acid A (THCA-A), Δ⁹-tetrahydrocannabinolic acid B (THCA-B), Δ⁹-tetrahydrocannabinol (THC), Δ⁹-tetrahydrocannabinolic acid-C₄ (THCA-C₄), Δ⁹-tetrahydrocannabinol-C₄ (THC-C₄), Δ⁹-tetrahydrocannabivarinic acid (THCVA), Δ⁹-tetrahydrocannabivarin (THCV), Δ⁹-tetrahydrocannabiorcolic acid (THCA-C₁), Δ⁹-tetrahydrocannabiorcol (THC-C₁), Δ⁷-cis-iso-tetrahydrocannabivarin, Δ⁸-tetrahydrocannabinolic acid (Δ⁸-THCA), Δ⁸-tetrahydrocannabinol (Δ⁸-THC), cannabicyclolic acid (CBLA), cannabicyclol (CBL), cannabicyclovarin (CBLV), cannabielsoic acid A (CBEA-A), cannabielsoic acid B (CBEA-B), cannabielsoin (CBE), cannabielsoinic acid, cannabicitranic acid, cannabinolic acid (CBNA), cannabinol (CBN), cannabinol methylether (CBNM), cannabinol-C₄, (CBN-C₄), cannabivarin (CBV), cannabinol-C₂ (CNB-C₂), cannabiorcol (CBN-C₁), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTV), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), and trihydroxy-delta-9-tetrahydrocannabinol (triOH-THC). Cannabinoids may be acidic or neutral depending on whether or not they have a carboxylic acid group present on the phenyl ring core of the cannabinoid. For example, THCA and CBDA are acidic cannabinoids, but THC and CBD are neutral cannabinoids. Herein, acidic cannabinoids may be referred to as an “acidic cannabinoid”, “cannabinoid acid”, “cannabinoid acid compound”, “acidic cannabinoid compound”, or “acid cannabinoid”. A cannabinoid lacking the carboxylic acid group on the phenyl ring core of the cannabinoid may be referred to as a “neutral cannabinoid” or “neutral cannabinoid compound”. In general, where the cannabinoids or cannabinoid derivatives can form salts, such salt forms are intended to be included in references to the cannabinoids or cannabinoid derivatives, unless otherwise specified or dictated by context.

As used herein, a “cannabinoid preparation” or “cannabinoid derivative preparation” may refer to the purified product (e.g., cannabinoid or cannabinoid derivative) obtained using the methods of the disclosure. The preparation may comprise any other components, but these components are not limited to excipients or carriers. A cannabinoid preparation may comprise an acidic cannabinoid, a neutral cannabinoid, or both an acidic cannabinoid and a neutral cannabinoid. A cannabinoid derivative preparation may comprise an acidic cannabinoid derivative, a neutral cannabinoid derivative, or both an acidic cannabinoid derivative and a neutral cannabinoid derivative. In some embodiments, the preparation contains one cannabinoid or cannabinoid derivative (e.g. the cannabinoid or cannabinoid derivative of interest). In some embodiments, the preparation is substantially free, or free of one or more other cannabinoids or cannabinoid derivatives. In some embodiments, the preparation contains a significant amount of one or more other cannabinoids or cannabinoid derivatives. In some embodiments, the preparation contains equal amounts of the cannabinoid or cannabinoid derivative of interest and one or more other cannabinoids or cannabinoid derivatives. In some embodiments, the preparation contains greater amounts of the cannabinoid or cannabinoid derivative of interest compared to the amounts of one or more other cannabinoids or cannabinoid derivatives.

As used herein, “impurity” or “impurities” may refer to components to be removed from the cannabinoid or cannabinoid derivative preparation. These include, without limitation, byproducts, cellular debris, cytoplasmic components, polypeptides, RNA, mRNA, membranes, cell wall components, media components, solvents, salts (e.g., Ca, Na, K, P, etc.), feed (e.g. hexanoic acid, pentanoic acid, butanoic acid), and carbon sources (e.g. sucrose, glucose, galactose).

As used herein, “byproducts” may refer to an undesired composition, component, compound, or molecule produced by the modified host cell (i.e., not the desired cannabinoid or cannabinoid derivative). These include, without limitation, hexanoyl triacetic acid lactone (HTAL), pentyl diacetic acid lactone (PDAL), olivetol, and olivetolic acid. In some embodiments, the byproduct may be produced through the action of one or more enzymes of the cannabinoid biosynthesis pathway expressed by the modified host cell.

An acyl-CoA compound as detailed herein may include compounds with the following structure:

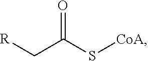

wherein R may be an unsubstituted fatty acid side chain or a fatty acid side chain substituted with or comprising one or more functional and/or reactive groups as disclosed herein (i.e., an acyl-CoA compound derivative).

As used herein, a hexanoyl CoA derivative, an acyl-CoA compound derivative, a cannabinoid derivative, an acidic cannabinoid derivative, a neutral cannabinoid derivative, or an olivetolic acid derivative may refer to hexanoyl CoA, an acyl-CoA compound, a cannabinoid, an acidic cannabinoid, a neutral cannabinoid, or olivetolic acid substituted with or comprising one or more functional and/or reactive groups. Functional groups may include, but are not limited to, azido, halo (e.g., chloride, bromide, iodide, fluorine), methyl, alkyl (including branched and straight chain alkyl groups), alkynyl, alkenyl, methoxy, alkoxy, acetyl, amino, carboxyl, carbonyl, oxo, ester, hydroxyl, thio (e.g., thiol), cyano, aryl, heteroaryl, cycloalkyl, cycloalkenyl, cycloalkylalkenyl, cycloalkylalkynyl, cycloalkenylalkyl, cycloalkenylalkenyl, cycloalkenylalkynyl, heterocyclylalkenyl, heterocyclylalkynyl, heteroarylalkenyl, heteroarylalkynyl, arylalkenyl, arylalkynyl, heterocyclyl, spirocyclyl, heterospirocyclyl, thioalkyl (or alkylthio), arylthio, heteroarylthio, sulfone, sulfonyl, sulfoxide, amido, alkylamino, dialkylamino, arylamino, alkylarylamino, diarylamino, N-oxide, imide, enamine, imine, oxime, hydrazone, nitrile, aralkyl, cycloalkylalkyl, haloalkyl, heterocyclylalkyl, heteroarylalkyl, nitro, thioxo, and the like. Suitable reactive groups may include, but are not necessarily limited to, azide, carboxyl, carbonyl, amine (e.g., alkyl amine (e.g., lower alkyl amine), aryl amine), halide, ester (e.g., alkyl ester (e.g., lower alkyl ester, benzyl ester), aryl ester, substituted aryl ester), cyano, thioester, thioether, sulfonyl halide, alcohol, thiol, succinimidyl ester, isothiocyanate, iodoacetamide, maleimide, hydrazine, alkynyl, alkenyl, and the like. A reactive group may facilitate covalent attachment of a molecule of interest. Suitable molecules of interest may include, but are not limited to, a detectable label; imaging agents; a toxin (including cytotoxins); a linker; a peptide; a drug (e.g., small molecule drugs); a member of a specific binding pair; an epitope tag; ligands for binding by a target receptor; tags to aid in purification; molecules that increase solubility; molecules that enhance bioavailability; molecules that increase in vivo half-life; molecules that target to a particular cell type; molecules that target to a particular tissue; molecules that provide for crossing the blood-brain barrier; molecules to facilitate selective attachment to a surface; and the like. Functional and reactive groups may be unsubstituted or substituted with one or more functional or reactive groups.

A cannabinoid derivative, an acidic cannabinoid derivative, a neutral cannabinoid derivative, or olivetolic acid derivative may also refer to a compound lacking one or more chemical moieties found in naturally-occurring cannabinoids, acidic cannabinoids, neutral cannabinoids, or olivetolic acid, yet retains the core structural features (e.g., cyclic core) of a naturally-occurring cannabinoid, acidic cannabinoid, neutral cannabinoid, or olivetolic acid. Such chemical moieties may include, but are not limited to, methyl, alkyl, alkenyl, methoxy, alkoxy, acetyl, carboxyl, carbonyl, oxo, ester, hydroxyl, and the like. In some embodiments, a cannabinoid derivative, an acidic cannabinoid derivative, a neutral cannabinoid derivative, or an olivetolic acid derivative may also comprise one or more of any of the functional and/or reactive groups described herein. Functional and reactive groups may be unsubstituted or substituted with one or more functional or reactive groups.

The term “nucleic acid” or “nucleic acids” used herein, may refer to a polymeric form of nucleotides of any length, either ribonucleotides or deoxynucleotides. Thus, this term may include, but is not limited to, single-, double-, or multi-stranded DNA or RNA, genomic DNA, cDNA, genes, synthetic DNA or RNA, DNA-RNA hybrids, or a polymer comprising purine and pyrimidine bases or other naturally-occurring, chemically or biochemically modified, non-naturally-occurring, or derivatized nucleotide bases.

The terms “peptide,” “polypeptide,” and “protein” may be used interchangeably herein, and may refer to a polymeric form of amino acids of any length, which can include coded and non-coded amino acids and chemically or biochemically modified or derivatized amino acids. The polypeptides disclosed herein may include full-length polypeptides, fragments of polypeptides, truncated polypeptides, fusion polypeptides, or polypeptides having modified peptide backbones. The polypeptides disclosed herein may also be variants differing from a specifically recited “reference” polypeptide (e.g., a wild-type polypeptide) by amino acid insertions, deletions, mutations, and/or substitutions.

As used herein, the term “heterologous” may refer to what is not normally found in nature. The term “heterologous nucleotide sequence” or the term “heterologous nucleic acid” may refer to a nucleic acid or nucleotide sequence not normally found in a given cell in nature. A heterologous nucleotide sequence may be: (a) foreign to its host cell (i.e., is “exogenous” to the cell); (b) naturally found in the host cell (i.e., “endogenous”) but present at an unnatural quantity in the cell (i.e., greater or lesser quantity than naturally found in the host cell); (c) be naturally found in the host cell but positioned outside of its natural locus; or (d) be naturally found in the host cell, but with introns removed or added. A heterologous nucleic acid may be: (a) foreign to its host cell (i.e., is “exogenous” to the cell); (b) naturally found in the host cell (i.e., “endogenous”) but present at an unnatural quantity in the cell (i.e., greater or lesser quantity than naturally found in the host cell); or (c) be naturally found in the host cell but positioned outside of its natural locus. In some embodiments, a heterologous nucleic acid may comprise a codon-optimized nucleotide sequence. A codon-optimized nucleotide sequence may be an example of a heterologous nucleotide sequence.

The term “heterologous enzyme” or “heterologous polypeptide” may refer to an enzyme or polypeptide that is not normally found in a given cell in nature. The term encompasses an enzyme or polypeptide that is: (a) exogenous to a given cell (i.e., encoded by a nucleic acid that is not naturally present in the host cell or not naturally present in a given context in the host cell); or (b) naturally found in the host cell (e.g., the enzyme or polypeptide is encoded by a nucleic acid that is endogenous to the cell) but that is produced in an unnatural amount (e.g., greater or lesser than that naturally found) in the host cell. For example, a heterologous polypeptide may include a mutated version of a polypeptide naturally occurring in a host cell.

As used herein, “substantially free” or “substantially purified” may refer to a cannabinoid or cannabinoid derivative preparation in which at least about 90% free of impurities and/or byproducts. In some embodiments, a “substantially free” cannabinoid or cannabinoid derivative preparation contains less than about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, about 0.1%, about 0.09%, about 0.08%, about 0.07%. about 0.06%, about 0.05%, about 0.04%, about 0.03%, about 0.02%, or about 0.01% impurities and/or byproducts.

In some embodiments, the relative amount and/or percent yield of the cannabinoid or cannabinoid derivative present before and/or after the processes or steps disclosed herein are measured using any appropriate method. In some embodiments, the relative amount and/or percent yield of the cannabinoid or cannabinoid derivative present before and/or after the processes or steps disclosed herein are measured using techniques including but not limited to, gas chromatography, mass spectrometry, gas chromatography combined with mass spectrometry (GC-MS), high performance liquid chromatography (HPLC), high performance liquid chromatography with UV or MS detection, or high pressure liquid chromatography (HPLC) with a diode array detector (DAD), or LC-MS. In some embodiments, the relative amount and/or percent yield of the cannabinoid or cannabinoid derivative present before and/or after the processes or steps disclosed herein is determined by LC-MS analysis. In certain such embodiments, each cannabinoid or cannabinoid derivative is identified by retention time, determined from an authentic standard, and multiple reaction monitoring (MRM) transition.

A “modified host cell” (also may be referred to as a “recombinant host cell”) may refer to a host cell into which has been introduced a nucleic acid (e.g., a heterologous nucleic acid), e.g., an expression vector or construct. For example, a modified eukaryotic host cell may be produced through introduction into a suitable eukaryotic host cell of a nucleic acid (e.g., a heterologous nucleic acid). The modified host cells described and used in the present disclosure may refer to the modified host cells disclosed in WO2018/200888 filed Apr. 27, 2018 and WO 2020/069214 filed Sep. 26, 2019, and PCT/US2020/051261 filed Sep. 17, 2020.

In some embodiments, conservative substitutions may be made in the amino acid sequence of a polypeptide without disrupting the three-dimensional structure or function of the polypeptide. Conservative substitutions may be accomplished by the skilled artisan by substituting amino acids with similar hydrophobicity, polarity, and R-chain length for one another. Additionally, by comparing aligned sequences of homologous proteins from different species, conservative substitutions may be identified by locating amino acid residues that have been mutated between species without altering the basic functions of the encoded proteins. The term “conservative amino acid substitution” may refer to the interchangeability in proteins of amino acid residues having similar side chains. For example, a group of amino acids having aliphatic side chains may consist of glycine, alanine, valine, leucine, and isoleucine; a group of amino acids having aliphatic-hydroxyl side chains may consist of serine and threonine; a group of amino acids having amide containing side chains may consist of asparagine and glutamine; a group of amino acids having aromatic side chains may consist of phenylalanine, tyrosine, and tryptophan; a group of amino acids having basic side chains may consist of lysine, arginine, and histidine; a group of amino acids having acidic side chains may consist of glutamate and aspartate; and a group of amino acids having sulfur containing side chains may consist of cysteine and methionine. Exemplary conservative amino acid substitution groups are: valine-leucine-isoleucine, phenylalanine-tyrosine, lysine-arginine, alanine-valine, and asparagine-glutamine.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

It must be noted that as used herein and in the appended claims, the singular forms “a,” “an,” and “the” may include plural referents unless the context clearly dictates otherwise. Thus, for example, reference to “a cannabinoid compound” or “cannabinoid” may include a plurality of such compounds and reference to “the modified host cell” may include reference to one or more modified host cells and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### Methods of Preparing Cannabinoid or Cannabinoid Derivative Preparations

Disclosed herein are methods of preparing a preparation comprising cannabinoid or cannabinoid derivative produced using a modified host cell (e.g., modified host cells such as disclosed in WO2018/200888 filed Apr. 27, 2018 and WO 2020/069214 filed Sep. 26, 2019, and PCT/US2020/051261 filed Sep. 17, 2020). The cannabinoid may be a neutral and/or an acidic cannabinoid and the cannabinoid derivative may be a neutral and/or an acidic cannabinoid derivative. In some embodiments, the cannabinoid is a neutral cannabinoid. In some embodiments, the cannabinoid is an acidic cannabinoid. In some embodiments, the cannabinoid derivative is a neutral cannabinoid derivative. In some embodiments, the cannabinoid derivative is an acidic cannabinoid derivative. In some embodiments, the disclosure provides for a method of preparing a cannabinoid preparation. In some embodiments, the disclosure provides for a method of preparing a cannabinoid derivative preparation. In some embodiments, the disclosure provides for a method of preparing an acidic cannabinoid preparation. In some embodiments, the disclosure provides for a method of preparing an acidic cannabinoid derivative preparation. In some embodiments, the disclosure provides for a method of preparing a neutral cannabinoid preparation. In some embodiments, the disclosure provides for a method of preparing a neutral cannabinoid derivative preparation.

The methods for preparing a cannabinoid or cannabinoid derivative preparation of the disclosure may include the steps in any combination or order as depicted in FIG. 1: 1) modified host cells in fermentation broth are washed to solubilize and separate impurities from the fermentation broth; 2) the fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) optionally, if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative is precipitated from an aqueous layer; and/or 7) the cannabinoid or cannabinoid derivative is crystallized. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative in the aqueous phase is subjected to chromatography (e.g. HPLC); and/or 6) the cannabinoid or cannabinoid derivative is precipitated from an aqueous layer. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative in the aqueous phase is subjected to chromatography (e.g. HPLC); 6) the cannabinoid or cannabinoid derivative is precipitated from an aqueous layer; and/or 7) the cannabinoid or cannabinoid derivative is crystallized. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative in the aqueous phase is subjected to chromatography (e.g. HPLC); and/or 6) the cannabinoid or cannabinoid derivative is crystallized. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative is precipitated from an aqueous layer; and/or 6) the cannabinoid or cannabinoid derivative is crystallized. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; and/or 6) the cannabinoid or cannabinoid derivative is precipitated from an aqueous layer. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; and/or 5) the cannabinoid or cannabinoid derivative is crystallized. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative in the aqueous phase is subjected to chromatography (e.g. HPLC); and/or 7) the cannabinoid or cannabinoid derivative is precipitated from an aqueous layer. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative in the aqueous phase is subjected to chromatography (e.g. HPLC); 7) the cannabinoid or cannabinoid derivative is precipitated from an aqueous layer; and/or 8) the cannabinoid or cannabinoid derivative is crystallized. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative in the aqueous phase is subjected to chromatography (e.g. HPLC); and/or 7) the cannabinoid or cannabinoid derivative is crystallized. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative in the aqueous phase is subjected to chromatography (e.g. HPLC); and/or 5) the cannabinoid or cannabinoid derivative is precipitated from an aqueous layer. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative in the aqueous phase is subjected to chromatography (e.g. HPLC); 5) the cannabinoid or cannabinoid derivative is precipitated from an aqueous layer; and/or 6) the cannabinoid or cannabinoid derivative is crystallized. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative in the aqueous phase is subjected to chromatography (e.g. HPLC); and/or 5) the cannabinoid or cannabinoid derivative is crystallized. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative is precipitated from an aqueous layer; and/or 5) the cannabinoid or cannabinoid derivative is crystallized. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; and/or 4) the cannabinoid or cannabinoid derivative is precipitated from an aqueous layer. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; and/or 4) the cannabinoid or cannabinoid derivative is crystallized. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 5) the cannabinoid or cannabinoid derivative in the aqueous phase is subjected to chromatography (e.g. HPLC); and/or 6) the cannabinoid or cannabinoid derivative is precipitated from an aqueous layer. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 5) the cannabinoid or cannabinoid derivative in the aqueous phase is subjected to chromatography (e.g. HPLC); 6) the cannabinoid or cannabinoid derivative is precipitated from an aqueous layer; and/or 7) the cannabinoid or cannabinoid derivative is crystallized. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 5) the cannabinoid or cannabinoid derivative in the aqueous phase is subjected to chromatography (e.g. HPLC); and/or 6) the cannabinoid or cannabinoid derivative is crystallized. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative in the aqueous phase is subjected to chromatography (e.g. HPLC); and/or 6) the cannabinoid or cannabinoid derivative is precipitated from an aqueous layer. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative in the aqueous phase is subjected to chromatography (e.g. HPLC); 6) the cannabinoid or cannabinoid derivative is precipitated from an aqueous layer; and/or 7) the cannabinoid or cannabinoid derivative is crystallized; and 8) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative in the aqueous phase is subjected to chromatography (e.g. HPLC); and/or 6) the cannabinoid or cannabinoid derivative is crystallized; and 7) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative is precipitated from an aqueous layer; and/or 6) the cannabinoid or cannabinoid derivative is crystallized; and 7) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; and/or 5) the cannabinoid or cannabinoid derivative is precipitated from an aqueous layer; and 6) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; and/or 5) the cannabinoid or cannabinoid derivative is crystallized; and 6) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative in the aqueous phase is subjected to chromatography (e.g. HPLC); and/or 7) the cannabinoid or cannabinoid derivative is precipitated from an aqueous layer; and 8) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative in the aqueous phase is subjected to chromatography (e.g. HPLC); 7) the cannabinoid or cannabinoid derivative is precipitated from an aqueous layer; and/or 8) the cannabinoid or cannabinoid derivative is crystallized; and 9) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative in the aqueous phase is subjected to chromatography (e.g. HPLC); and/or 7) the cannabinoid or cannabinoid derivative is crystallized; and 8) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative in the aqueous phase is subjected to chromatography (e.g. HPLC); and/or 5) the cannabinoid or cannabinoid derivative is precipitated from an aqueous layer; and 6) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative in the aqueous phase is subjected to chromatography (e.g. HPLC); 5) the cannabinoid or cannabinoid derivative is precipitated from an aqueous layer; and/or 6) the cannabinoid or cannabinoid derivative is crystallized; and 7) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative in the aqueous phase is subjected to chromatography (e.g. HPLC); and/or 5) the cannabinoid or cannabinoid derivative is crystallized; and 6) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative is precipitated from an aqueous layer; and/or 5) the cannabinoid or cannabinoid derivative is crystallized; and 6) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; and/or 4) the cannabinoid or cannabinoid derivative is precipitated from an aqueous layer; and 5) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; and/or 4) the cannabinoid or cannabinoid derivative is crystallized; and 5) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 5) the cannabinoid or cannabinoid derivative in the aqueous phase is subjected to chromatography (e.g. HPLC); and/or 6) the cannabinoid or cannabinoid derivative is precipitated from an aqueous layer; and 7) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 5) the cannabinoid or cannabinoid derivative in the aqueous phase is subjected to chromatography (e.g. HPLC); 6) the cannabinoid or cannabinoid derivative is precipitated from an aqueous layer; and/or 7) the cannabinoid or cannabinoid derivative is crystallized; and 8) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the steps as depicted in FIG. 1 are performed in any order: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 5) the cannabinoid or cannabinoid derivative in the aqueous phase is subjected to chromatography (e.g. HPLC); and/or 6) the cannabinoid or cannabinoid derivative is crystallized; and 7) recovering the resulting cannabinoid or cannabinoid derivative preparation, wherein the cannabinoid or cannabinoid derivative preparation comprises at least 85 weight % of the neutral cannabinoid or neutral cannabinoid derivative. These steps may be performed in any combination or order. One or more of these steps may be omitted or may be performed multiple times (e.g., twice, three times). In some embodiments, the steps include a measurement of purification and/or yield before any one step. In some embodiments, the steps include a measurement of purification and/or yield before one or more steps. In some embodiments, the steps include a measurement of purification and/or yield before each step.

In some embodiments, the disclosure provides for a method of preparing a cannabinoid or cannabinoid derivative preparation, said method comprising the steps of: 1) extracting an acidic cannabinoid or acidic cannabinoid derivative from a fermentation broth into an emollient phase; 2) extracting the acidic cannabinoid or acidic cannabinoid derivative in emollient into an aqueous phase; 3) decarboxylating the acidic cannabinoid or acidic cannabinoid derivative in the aqueous phase to afford a neutral cannabinoid or neutral cannabinoid derivative; 4) crystallizing the neutral cannabinoid or neutral cannabinoid derivative; and 5) recovering the resulting cannabinoid or cannabinoid derivative preparation, wherein the cannabinoid or cannabinoid derivative preparation comprises at least 85 weight % of the neutral cannabinoid or neutral cannabinoid derivative. In some embodiments, the method comprises a step of washing the fermentation broth before extracting the acidic cannabinoid or acidic cannabinoid derivative into the emollient phase. In some embodiments, the method comprises a step of solubilizing the crystallized neutral cannabinoid or neutral cannabinoid derivative and recrystallizing the neutral cannabinoid or neutral cannabinoid derivative.

In certain embodiments, the disclosure provides for a method preparing a cannabinoid or cannabinoid derivative preparation, said method comprising: extracting an acidic cannabinoid or acidic cannabinoid derivative from a fermentation broth using an emollient phase and recovering the resulting cannabinoid or cannabinoid derivative preparation, wherein the cannabinoid or cannabinoid derivative preparation comprises at least 85 weight % of cannabinoid or cannabinoid derivative, wherein the cannabinoid or cannabinoid derivative is a neutral cannabinoid, a neutral cannabinoid derivative, the acidic cannabinoid, or the acidic cannabinoid derivative.

In some embodiments, the disclosure provides for a method of preparing a cannabinoid or cannabinoid derivative preparation, said method comprising the steps of: 1) extracting a neutral cannabinoid or neutral cannabinoid derivative from a fermentation broth into an emollient phase; 2) extracting the neutral cannabinoid or neutral cannabinoid derivative in emollient into an aqueous phase; 3) crystallizing the neutral cannabinoid or neutral cannabinoid derivative; and 4) recovering the resulting cannabinoid or cannabinoid derivative preparation, wherein the cannabinoid or cannabinoid derivative preparation comprises at least 85 weight % of the neutral cannabinoid or neutral cannabinoid derivative. In some embodiments, the method comprises a step of washing the fermentation broth before extracting the neutral cannabinoid or neutral cannabinoid derivative into the emollient phase. In some embodiments, the method comprises a step of solubilizing the crystallized neutral cannabinoid or neutral cannabinoid derivative and recrystallizing the neutral cannabinoid or neutral cannabinoid derivative.

In certain embodiments, the disclosure provides for a method preparing a cannabinoid or cannabinoid derivative preparation, said method comprising: extracting a neutral cannabinoid or neutral cannabinoid derivative from a fermentation broth using an emollient phase and recovering the resulting cannabinoid or cannabinoid derivative preparation, wherein the cannabinoid or cannabinoid derivative preparation comprises at least 85 weight % of cannabinoid or cannabinoid derivative, wherein the cannabinoid or cannabinoid derivative is a neutral cannabinoid or a neutral cannabinoid derivative.

As used herein, fermentation broth may refer to the fermentation broth, whole fermentation broth, cell culture (e.g., whole cell culture or whole cell broth), fermentation liquid, or yeast fermentation broth. In some embodiments, fermentation broth comprises modified host cells, a culture medium, or both modified host cells and culture medium. In some embodiments, fermentation broth comprises modified host cells. In some embodiments, fermentation broth comprises a culture medium. In some embodiments, fermentation broth comprises both modified host cells and culture medium. In some embodiments, fermentation broth comprises modified host cells; a culture medium; modified host cell lysate; both modified host cells and culture medium; both modified host cell lysate and culture medium; or modified host cells, modified host cell lysate, and culture medium. In some embodiments, fermentation broth comprises both modified host cell lysate and culture medium. In some embodiments, fermentation broth comprises modified host cells, modified host cell lysate, and culture medium. In some embodiments, the modified host cells are yeast cells.

In some embodiments of the disclosure, the modified host cells producing the cannabinoids or cannabinoid derivatives (e.g. an acidic cannabinoid, acidic cannabinoid derivative, neutral cannabinoid, or neutral cannabinoid derivative) are those described in WO2018/200888 filed Apr. 27, 2018 and WO 2020/069214 filed Sep. 26, 2019, and PCT/US2020/051261 filed Sep. 17, 2020, the contents of which are incorporated by reference in their entirety for all purposes. The fermentation culture (fermentation broth) of these cells may contain the cannabinoid or cannabinoid derivative (e.g., an acidic cannabinoid, acidic cannabinoid derivative, neutral cannabinoid, or neutral cannabinoid derivative) along with certain impurities, including but not limited to, cellular debris, salts, components of the fermentation culture, and unwanted byproducts, such as feed (e.g. hexanoic acid, pentanoic acid, butanoic acid), carbon sources (e.g. sucrose, glucose, galactose), hexanoyl triacetic acid lactone (HTAL), pentyl diacetic acid lactone (PDAL), olivetol, and olivetolic acid. The modified host cells may be modified to maximize the production of the cannabinoid or cannabinoid derivative of interest (e.g., an acidic cannabinoid, acidic cannabinoid derivative, neutral cannabinoid, or neutral cannabinoid derivative), and to minimize the production of unwanted byproducts. In some embodiments, the cannabinoid or cannabinoid derivative recovered in the methods or preparations of the disclosure is in the form of a salt. In certain such embodiments, the salt is a pharmaceutically acceptable salt. In some embodiments, the salt is an acceptable salt. In some embodiments, the cannabinoid or cannabinoid derivative recovered in the methods or preparations of the disclosure is not in the form of a salt.

In some embodiments, the fermentation broth contains about 1% to about 50% w/v of the cannabinoid or cannabinoid derivative (e.g. an acidic cannabinoid, acidic cannabinoid derivative, neutral cannabinoid, or neutral cannabinoid derivative). In some embodiments, the fermentation broth contains about 50%, about 49%, about 48%, about 47%, about 46%, about 45%, about 44%, about 43%, about 42%, about 41%, about 40%, about 39%, about 38%, about 37%, about 36%, about 35%, about 34%, about 33%, about 32%, about 31%, about 30%, about 29%, about 28%, about 27%, about 26%, about 25%, about 24%, about 23%, about 22%, about 21%, about 20%, about 19%, about 18%, about 17%, about 16%, about 15%, about 14%, about 13%, about 12%, about 11%, about 10%, about 9%, about 8%, about 7%, about 6%, about 5%, about 4%, about 3%, about 2%, or about 1% w/v of the cannabinoid or cannabinoid derivative (e.g. an acidic cannabinoid, acidic cannabinoid derivative, neutral cannabinoid, or neutral cannabinoid derivative).

In some embodiments, the cannabinoid or cannabinoid derivative is present in the fermentation broth, whole fermentation broth, or preparation after any of the downstream processing steps disclosed herein at a concentration greater than about 0.1 mg/L. In some embodiments, the cannabinoid or cannabinoid derivative is present in the fermentation broth, whole fermentation broth, or preparation after any of the downstream processing steps disclosed herein at a concentration of about 0.1 mg/L to about 500 g/L. In some embodiments, the cannabinoid or cannabinoid derivative is present in the fermentation broth, whole fermentation broth, or preparation after any of the downstream processing steps disclosed herein at a concentration of greater than about 500 g/L. In some embodiments, the cannabinoid or cannabinoid derivative is present in the fermentation broth, whole fermentation broth, or preparation after any of the downstream processing steps disclosed herein at a concentration of about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.1 mg/L, about 1.2 mg/L, about 1.3 mg/L, about 1.4 mg/L, about 1.5 mg/L, about 1.6 mg/L, about 1.7 mg/L, about 1.8 mg/L, about 1.9 mg/L, about 2.0 mg/L, about 3 mg/L, about 4.0 mg/L, about 5.0 mg/L, about 6.0 mg/L, about 7.0 mg/L, about 8.0 mg/L, about 9.0 mg/L, about 10 mg/L, about 11 mg/L, about 12 mg/L, about 13 mg/L, about 14 mg/L, about 15 mg/L, about 16 mg/L, about 17 mg/L, about 18 mg/L, about 19 mg/L, about 20 mg/L, about 21 mg/L, about 22 mg/L, about 23 mg/L, about 24 mg/L, about 25 mg/L, about 26 mg/L, about 27 mg/L, about 28 mg/L, about 29 mg/L, about 30 mg/L, about 31 mg/L, about 32 mg/L, about 33 mg/L, about 34 mg/L, about 35 mg/L, about 36 mg/L, about 37 mg/L, about 38 mg/L, about 39 mg/L, about 40 mg/L, about 41 mg/L, about 42 mg/L, about 43 mg/L, about 44 mg/L, about 45 mg/L, about 46 mg/L, about 47 mg/L, about 48 mg/L, about 49 mg/L, about 50 mg/L, about 51 mg/L, about 52 mg/L, about 53 mg/L, about 54 mg/L, about 55 mg/L, about 56 mg/L, about 57 mg/L, about 58 mg/L, about 59 mg/L, about 60 mg/L, about 61 mg/L, about 62 mg/L, about 63 mg/L, about 64 mg/L, about 65 mg/L, about 66 mg/L, about 67 mg/L, about 68 mg/L, about 69 mg/L, about 70 mg/L, about 71 mg/L, about 72 mg/L, about 73 mg/L, about 74 mg/L, about 75 mg/L, about 76 mg/L, about 77 mg/L, about 78 mg/L, about 79 mg/L, about 80 mg/L, about 81 mg/L, about 82 mg/L, about 83 mg/L, about 84 mg/L, about 85 mg/L, about 86 mg/L, about 87 mg/L, about 88 mg/L, about 89 mg/L, about 90 mg/L, about 91 mg/L, about 92 mg/L, about 93 mg/L, about 94 mg/L, about 95 mg/L, about 96 mg/L, about 97 mg/L, about 98 mg/L, about 99 mg/L, about 100 mg/L, about 200 mg/L, about 300 mg/L, about 400 mg/L, about 500 mg/L, about 600 mg/L, about 700 mg/L, about 800 mg/L, about 900 mg/L, about lg/L, about 5 g/L, about 10 g/L, about 15 g/L, about 20 g/L, about 30 g/L, about 40 g/L, about 50 g/L, about 60 g/L, about 70 g/L, about 80 g/L, about 90 g/L, about 100 g/L, about 200 g/L, about 300 g/L, about 400 g/L, about 500 g/L, or greater than about 500 g/L.

In some embodiments, the cannabinoid or cannabinoid derivative is present in the fermentation broth, whole fermentation broth, or preparation after any of the downstream processing steps disclosed herein at a concentration of about 0.1 mg/L to about 500 g/L and is free of impurities and byproducts. In some embodiments, the cannabinoid or cannabinoid derivative is present in the fermentation broth, whole fermentation broth, or preparation after any of the downstream processing steps disclosed herein at a concentration of about 0.1 mg/L to about 500 g/L and is substantially free of impurities and byproducts. In some embodiments, the cannabinoid or cannabinoid derivative is present in the fermentation broth, whole fermentation broth, or preparation after any of the downstream processing steps disclosed herein at a concentration of about 0.1 mg/L to about 500 g/L and contains less than 5% impurities and byproducts. In some embodiments, the cannabinoid or cannabinoid derivative is present in the fermentation broth, whole fermentation broth, or preparation after any of the downstream processing steps disclosed herein at a concentration of greater than about 500 g/L and is free of impurities and byproducts. In some embodiments, the cannabinoid or cannabinoid derivative is present in the fermentation broth, whole fermentation broth, or preparation after any of the downstream processing steps disclosed herein at a concentration of greater than about 500 g/L and is substantially free of impurities and byproducts. In some embodiments, the cannabinoid or cannabinoid derivative is present in the fermentation broth, whole fermentation broth, or preparation after any of the downstream processing steps disclosed herein at a concentration of greater than about 500 g/L and contains less than 5% impurities and byproducts. In some embodiments, the cannabinoid or cannabinoid derivative is present in the fermentation broth, whole fermentation broth, or preparation after any of the downstream processing steps disclosed herein at a concentration of about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.1 mg/L, about 1.2 mg/L, about 1.3 mg/L, about 1.4 mg/L, about 1.5 mg/L, about 1.6 mg/L, about 1.7 mg/L, about 1.8 mg/L, about 1.9 mg/L, about 2.0 mg/L, about 3 mg/L, about 4.0 mg/L, about 5.0 mg/L, about 6.0 mg/L, about 7.0 mg/L, about 8.0 mg/L, about 9.0 mg/L, about 10 mg/L, about 11 mg/L, about 12 mg/L, about 13 mg/L, about 14 mg/L, about 15 mg/L, about 16 mg/L, about 17 mg/L, about 18 mg/L, about 19 mg/L, about 20 mg/L, about 21 mg/L, about 22 mg/L, about 23 mg/L, about 24 mg/L, about 25 mg/L, about 26 mg/L, about 27 mg/L, about 28 mg/L, about 29 mg/L, about 30 mg/L, about 31 mg/L, about 32 mg/L, about 33 mg/L, about 34 mg/L, about 35 mg/L, about 36 mg/L, about 37 mg/L, about 38 mg/L, about 39 mg/L, about 40 mg/L, about 41 mg/L, about 42 mg/L, about 43 mg/L, about 44 mg/L, about 45 mg/L, about 46 mg/L, about 47 mg/L, about 48 mg/L, about 49 mg/L, about 50 mg/L, about 51 mg/L, about 52 mg/L, about 53 mg/L, about 54 mg/L, about 55 mg/L, about 56 mg/L, about 57 mg/L, about 58 mg/L, about 59 mg/L, about 60 mg/L, about 61 mg/L, about 62 mg/L, about 63 mg/L, about 64 mg/L, about 65 mg/L, about 66 mg/L, about 67 mg/L, about 68 mg/L, about 69 mg/L, about 70 mg/L, about 71 mg/L, about 72 mg/L, about 73 mg/L, about 74 mg/L, about 75 mg/L, about 76 mg/L, about 77 mg/L, about 78 mg/L, about 79 mg/L, about 80 mg/L, about 81 mg/L, about 82 mg/L, about 83 mg/L, about 84 mg/L, about 85 mg/L, about 86 mg/L, about 87 mg/L, about 88 mg/L, about 89 mg/L, about 90 mg/L, about 91 mg/L, about 92 mg/L, about 93 mg/L, about 94 mg/L, about 95 mg/L, about 96 mg/L, about 97 mg/L, about 98 mg/L, about 99 mg/L, about 100 mg/L, about 200 mg/L, about 300 mg/L, about 400 mg/L, about 500 mg/L, about 600 mg/L, about 700 mg/L, about 800 mg/L, about 900 mg/L, about lg/L, about 5 g/L, about 10 g/L, about 15 g/L, about 20 g/L, about 30 g/L, about 40 g/L, about 50 g/L, about 60 g/L, about 70 g/L, about 80 g/L, about 90 g/L, about 100 g/L, about 200 g/L, about 300 g/L, about 400 g/L, about 500 g/L, or greater than about 500 g/L and contains about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% impurities and byproducts. In some embodiments, the byproducts and/or impurities may include, but are not limited to, hexanoyl triacetic acid lactone (HTAL), pentyl diacetic acid lactone (PDAL), olivetol, olivetolic acid, and hexanoic acid.

In some embodiments, the fermentation broth, whole fermentation broth, or preparation after any of the downstream processing steps disclosed herein contains byproducts measured by weight percentage relative to the cannabinoid or cannabinoid derivative. In some embodiments, the total amount of byproducts in the fermentation broth is between about 80% to about 1% weight relative to the cannabinoid or cannabinoid derivative (e.g., an acidic cannabinoid, acidic cannabinoid derivative, neutral cannabinoid, or neutral cannabinoid derivative). In some embodiments, the byproducts and/or impurities in the fermentation broth, whole fermentation broth, or preparation after any of the downstream processing steps disclosed herein may include, but are not limited to hexanoyl triacetic acid lactone (HTAL), pentyl diacetic acid lactone (PDAL), olivetol, olivetolic acid, and hexanoic acid. In some embodiments, the amount of HTAL in the fermentation broth, whole fermentation broth, or preparation after any of the downstream processing steps disclosed herein is between about 0.01% and about 30% weight relative to the cannabinoid or cannabinoid derivative (e.g., an acidic cannabinoid, acidic cannabinoid derivative, neutral cannabinoid, or neutral cannabinoid derivative). In some embodiments, the amount of HTAL in the fermentation broth, whole fermentation broth, or preparation after any of the downstream processing steps disclosed herein is less than about 5.0% weight relative to the cannabinoid or cannabinoid derivative (e.g., an acidic cannabinoid, acidic cannabinoid derivative, neutral cannabinoid, or neutral cannabinoid derivative). In some embodiments, the amount of PDAL in the fermentation broth, whole fermentation broth, or preparation after any of the downstream processing steps disclosed herein is between about 0.01% and about 30% weight relative to the cannabinoid or cannabinoid derivative (e.g., an acidic cannabinoid, acidic cannabinoid derivative, neutral cannabinoid, or neutral cannabinoid derivative). In some embodiments, the amount of PDAL in the fermentation broth, whole fermentation broth, or preparation after any of the downstream processing steps disclosed herein is less than about 5% weight relative to the cannabinoid or cannabinoid derivative (e.g., an acidic cannabinoid, acidic cannabinoid derivative, neutral cannabinoid, or neutral cannabinoid derivative). In some embodiments, the amount of olivetol in the fermentation broth, whole fermentation broth, or preparation after any of the downstream processing steps disclosed herein is between about 1.0% to about 50% relative to the cannabinoid or cannabinoid derivative (e.g., an acidic cannabinoid, acidic cannabinoid derivative, neutral cannabinoid, or neutral cannabinoid derivative). In some embodiments, the amount of olivetol in the fermentation broth, whole fermentation broth, or preparation after any of the downstream processing steps disclosed herein is between about 20% to about 40% relative to the cannabinoid or cannabinoid derivative (e.g., an acidic cannabinoid, acidic cannabinoid derivative, neutral cannabinoid, or neutral cannabinoid derivative). In some embodiments, the amount of olivetol in the fermentation broth, whole fermentation broth, or preparation after any of the downstream processing steps disclosed herein is between about 25% to about 30% relative to the cannabinoid or cannabinoid derivative (e.g., an acidic cannabinoid, acidic cannabinoid derivative, neutral cannabinoid, or neutral cannabinoid derivative). In some embodiments, the amount of olivetolic acid in the fermentation broth, whole fermentation broth, or preparation after any of the downstream processing steps disclosed herein is between about 1.0% to about 60% relative to the cannabinoid or cannabinoid derivative (e.g., an acidic cannabinoid, acidic cannabinoid derivative, neutral cannabinoid, or neutral cannabinoid derivative). In some embodiments, the amount of olivetolic acid in the fermentation broth, whole fermentation broth, or preparation after any of the downstream processing steps disclosed herein is between about 50% to about 60% relative to the cannabinoid or cannabinoid derivative (e.g., an acidic cannabinoid, acidic cannabinoid derivative, neutral cannabinoid, or neutral cannabinoid derivative). In some embodiments, the amount of hexanoic acid in the fermentation broth, whole fermentation broth, or preparation after any of the downstream processing steps disclosed herein is between about 1.0% to about 60% relative to the cannabinoid or cannabinoid derivative (e.g., an acidic cannabinoid, acidic cannabinoid derivative, neutral cannabinoid, or neutral cannabinoid derivative). In some embodiments, the amount of hexanoic acid in the fermentation broth, whole fermentation broth, or preparation after any of the downstream processing steps disclosed herein is between about 30% to about 60% relative to the cannabinoid or cannabinoid derivative (e.g., an acidic cannabinoid, acidic cannabinoid derivative, neutral cannabinoid, or neutral cannabinoid derivative). In some embodiments, the amount of cannabigerovarinic acid (CBGVA) in the fermentation broth, whole fermentation broth, or preparation after any of the downstream processing steps disclosed herein is between about 1.0% to about 60% relative to the cannabinoid or cannabinoid derivative (e.g., an acidic cannabinoid, acidic cannabinoid derivative, neutral cannabinoid, or neutral cannabinoid derivative). In some embodiments, the amount of CBGVA in the fermentation broth, whole fermentation broth, or preparation after any of the downstream processing steps disclosed herein is between about 30% to about 60% relative to the cannabinoid or cannabinoid derivative (e.g., an acidic cannabinoid, acidic cannabinoid derivative, neutral cannabinoid, or neutral cannabinoid derivative).

### Fermentation Broth Washing

In some embodiments, the produced cannabinoid or cannabinoid derivative (e.g., an acidic cannabinoid, acidic cannabinoid derivative, neutral cannabinoid, or neutral cannabinoid derivative) may be secreted and/or passively diffuse into the fermentation broth (e.g., whole fermentation broth, culture media, or fermentation media), or the produced cannabinoid or cannabinoid derivative may be associated with the cell wall and/or cell membrane. As the cannabinoid or cannabinoid derivative (e.g., an acidic cannabinoid, acidic cannabinoid derivative, neutral cannabinoid, or neutral cannabinoid derivative) is thus in the fermentation broth or adhered to the cell, this avoids the requirement to lyse the modified host cells to extract or purify the cannabinoid or cannabinoid derivative (e.g., an acidic cannabinoid, acidic cannabinoid derivative, neutral cannabinoid, or neutral cannabinoid derivative), however a certain percentage of the modified host cells may lyse during the extraction/purification process. In some embodiments, the modified host cells are lysed to extract/purify the cannabinoid or cannabinoid derivative (e.g., an acidic cannabinoid, acidic cannabinoid derivative, neutral cannabinoid, or neutral cannabinoid derivative).

In some aspects of the disclosure, the fermentation broth (e.g., whole fermentation broth, cell culture, or fermentation broth) is washed to solubilize and separate impurities and/or byproducts from the fermentation broth. In some embodiments, the fermentation broth is washed from zero to 10 times. In some embodiments, the fermentation broth is washed one time. In some embodiments, the fermentation broth is washed two times, in some embodiments, the fermentation broth is washed three times. In some embodiments, the fermentation broth is washed four times. In some embodiments, the fermentation broth is washed five times. In some embodiments, the fermentation broth is washed six times. In some embodiments, the fermentation broth is washed seven times. In some embodiments, the fermentation broth is washed eight times. In some embodiments, the fermentation broth is washed nine times. In some embodiments, the fermentation broth is washed 10 times. In some embodiments, the fermentation broth is not washed.

Any appropriate washing media may be used. In some embodiments, the washing media is an aqueous phase. In some embodiments, the washing media is water, distilled water, saline, or phosphate buffered saline (PBS). In some embodiments, the washing media is at about 25° C. In some embodiments, the washing media is ice cold. In some embodiments, the temperature of the washing media is between about 4° C. to about 40° C.

In some embodiments, the fermentation broth washing is performed at a pH between about 4.0 and about 10.0. In some embodiments, the fermentation broth washing is performed at a pH between about 5.0 and about 9.0. In some embodiments, the fermentation broth washing is performed at a pH of about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, or about 10.0. In some embodiments, the fermentation broth washing is performed at a pH between about 7.0 and about 8.0. In some embodiments, the fermentation broth washing is performed at a pH of about 7.1, about 7.2, about 7.3, about 7.4, about 7.5, about 7.6, about 7.7, about 7.8, about 7.9, about 7.10, about 7.11, about 7.12, about 7.13, about 7.14, about 7.15, about 7.16, about 7.17, about 7.18, about 7.19, about 7.20, about 7.21, about 7.22, about 7.23, about 7.24, about 7.25, about 7.26, about 7.27, about 7.28, about 7.29, about 7.30, about 7.31, about 7.32, about 7.33, about 7.34, about 7.35, about 7.36, about 7.37, about 7.38, about 7.39, about 7.40, about 7.41, about 7.42, about 7.43, about 7.44, about 7.45, about 7.46, about 7.47, about 7.48, about 7.49, about 7.50, about 7.51, about 7.52, about 7.53, about 7.54, about 7.55, about 7.56, about 7.57, about 7.58, about 7.59, about 7.60, about 7.61, about 7.62, about 7.63, about 7.64, about 7.65, about 7.66, about 7.67, about 7.68, about 7.69, about 7.70, about 7.71, about 7.72, about 7.73, about 7.74, about 7.75, about 7.76, about 7.77, about 7.78, about 7.79, about 7.80, about 7.81, about 7.82, about 7.83, about 7.84, about 7.85, about 7.86, about 7.87, about 7.88, about 7.89, about 7.80, about 7.81, about 7.82, about 7.83, about 7.84, about 7.85, about 7.86, about 7.87, about 7.88, about 7.89, about 7.90, about 7.91, about 7.92, about 7.93, about 7.94, about 7.95, about 7.96, about 7.97, about 7.98, about 7.99, or about 8.0.

In some embodiments, the fermentation broth washing includes centrifugation to separate the modified host cell biomass from the media. In some embodiments, the fermentation broth is centrifuged for about 5 minutes to about 40 minutes. In some embodiments, the fermentation broth is centrifuged for about 5 minutes, about 10 minutes, about 15 minutes, about 20 minutes, about 25 minutes, about 30 minutes, about 35 minutes, or about 40 minutes. In some embodiments, the cell pellet is resuspended in a fresh aqueous media. The suspension of the washed modified host cells in the fresh aqueous media may be centrifuged again, or may be used in another step of the downstream processing system disclosed herein.

In some embodiments, the fermentation broth washing removes impurities and/or byproducts. In some embodiments, the fermentation broth washing removes impurities and/or byproducts without losing significant amounts of the desired cannabinoid or cannabinoid derivative (e.g., an acidic cannabinoid, acidic cannabinoid derivative, neutral cannabinoid, or neutral cannabinoid derivative). In some embodiments, the fermentation broth washing removes about 50% to about 100% of CBGVA weight relative to the cannabinoid or cannabinoid derivative (e.g., an acidic cannabinoid, acidic cannabinoid derivative, neutral cannabinoid, or neutral cannabinoid derivative). In some embodiments, the fermentation broth washing removes about 50% to about 100% of HTAL weight relative to the cannabinoid or cannabinoid derivative (e.g., an acidic cannabinoid, acidic cannabinoid derivative, neutral cannabinoid, or neutral cannabinoid derivative). In some embodiments, the fermentation broth washing removes about 50% to about 100% olivetolic acid weight relative to the cannabinoid or cannabinoid derivative (e.g., an acidic cannabinoid, acidic cannabinoid derivative, neutral cannabinoid, or neutral cannabinoid derivative). In some embodiments, the fermentation broth washing removes about 50% to about 100% PDAL weight relative to the cannabinoid or cannabinoid derivative (e.g., an acidic cannabinoid, acidic cannabinoid derivative, neutral cannabinoid, or neutral cannabinoid derivative). In some embodiments, the fermentation broth washing removes about 10% to about 50% olivetol weight relative to the cannabinoid or cannabinoid derivative (e.g., an acidic cannabinoid, acidic cannabinoid derivative, neutral cannabinoid, or neutral cannabinoid derivative). In some embodiments, the fermentation broth washing removes about 0% to about 20% of the cannabinoid or cannabinoid derivative in the fermentation broth (e.g., an acidic cannabinoid, acidic cannabinoid derivative, neutral cannabinoid, or neutral cannabinoid derivative).

In some embodiments, the washed fermentation broth retains at least 40% of the cannabinoid or cannabinoid derivative (e.g., an acidic cannabinoid, acidic cannabinoid derivative, neutral cannabinoid, or neutral cannabinoid derivative). In some embodiments, the washed fermentation broth retains about 40% to about 100% of the cannabinoid or cannabinoid derivative (e.g., an acidic cannabinoid, acidic cannabinoid derivative, neutral cannabinoid, or neutral cannabinoid derivative). In some embodiments, the washed fermentation broth retains about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, or about 100% of the cannabinoid or cannabinoid derivative (e.g., an acidic cannabinoid, acidic cannabinoid derivative, neutral cannabinoid, or neutral cannabinoid derivative). In some embodiments, the cannabinoid or cannabinoid derivative is an acidic cannabinoid or acidic cannabinoid derivative. In some embodiments, the acidic cannabinoid or acidic cannabinoid derivative is cannabigerolic acid (CBGA), tetrahydrocannabinolic acid (THCA), or cannabidiolic acid (CBDA).

In some embodiments, the fermentation broth is washed at a pH of 7.67 and removes less than or about 10% of the cannabinoid or cannabinoid derivative (e.g., an acidic cannabinoid, acidic cannabinoid derivative, neutral cannabinoid, or neutral cannabinoid derivative), about 85% of the HTAL and olivetolic acid, about 75% of the PDAL, and about 25% of the olivetol. In some embodiments, the cannabinoid or cannabinoid derivative is an acidic cannabinoid or acidic cannabinoid derivative. In some embodiments, the acidic cannabinoid or acidic cannabinoid derivative is cannabigerolic acid (CBGA).

### Extraction of Cannabinoids or Cannabinoid Derivatives

In some aspects of the present disclosure, the cannabinoid or cannabinoid derivative (e.g., an acidic cannabinoid, acidic cannabinoid derivative, neutral cannabinoid, or neutral cannabinoid derivative) is extracted from the fermentation broth if the washing step is omitted (e.g., whole fermentation broth) or the washed fermentation broth if the washing step is included, into an emollient phase. In some embodiments, the emollient is a polar emollient. This extraction leaves behind most of the impurities and/or byproducts from the fermentation broth or the washed fermentation broth while extracting all or most of the cannabinoid or cannabinoid derivative (e.g., an acidic cannabinoid, acidic cannabinoid derivative, neutral cannabinoid, or neutral cannabinoid derivative) into the emollient phase.

As used herein, “emollient” may refer to a natural or synthetic substance in which the desired cannabinoid or cannabinoid derivative (e.g., an acidic cannabinoid, acidic cannabinoid derivative, neutral cannabinoid, or neutral cannabinoid derivative) is solubilized. The emollient may be any appropriate substance. In some embodiments, the emollient may be approved for administration to a subject. In some embodiments, the emollient is an oil. In some embodiments, the emollient is a polar emollient. In some embodiments, the emollient is a non-polar emollient. In some embodiments, the emollient is selected from, but not limited to, medium-chain triglycerides (MCT), oils, essential oils (e.g., R-limonene, D-limonene, neem oil, lavender oil, peppermint oil, anise oil, rosemary oil, sage oil, hibiscus oil, berries oil (any type), menthol, capsaicin, grape seed oil, pumpkin oil, hemp oil, terpenes, or terpene-less), mineral oil, paraffinic oils, phospholipids, polar lipids (squalenes, spingomelines), waxes (e.g., animal or plant waxes such as carnauba wax or beeswax), vegetable oils (e.g., olive oil, soybean oil, canola oil, cotton oil, palmolein, sunflower oil, corn oil, rapeseed oil, grape seeds oil, hemp oil, pomegranate oil, avocado oil, peppermint oil, tomato oil), triglycerides, glycerides, fatty acids and esters of fatty acids (e.g., isopropyl myristate, oleyl lactate, coco caprylocaprate, hexyl laurate, oleic acid, linoleic acid, ethyl oleate, ethyl laurate, ethyl hexyl laurate, ethyl hexyl oleate, lauryl acetate, lauryl lactate), fatty alcohols (e.g., oleyl alcohol, linoleyl alcohol, cetyl alcohol), fatty amines (e.g., oleyl amine), liquid hydrocarbons, or solvents (e.g. petroleum ether, naphtha, ethyl acetate, butyl acetate, butyl butyrate, methyl tert-butyl ether (MBTE), hexane, heptanes, nonane, decane, dodecane, toluene, methyl isobutyl ketone (MIBK), ethanol, methanol, isopropanol, benzyl alcohol, glycerol, triacetin), and mixtures thereof. In some embodiments, the emollient is selected from, but not limited to, olive oil, soybean oil, canola oil, cotton oil, palmolein, sunflower oil, corn oil, rapeseed oil, grape seeds oil, hemp oil, pomegranate oil, avocado oil, peppermint oil, tomato oil, isopropyl myristate, oleyl lactate, coco caprylocaprate, hexyl laurate, oleyl amine, oleic acid, oleyl alcohol, linoleic acid, linoleyl alcohol, ethyl oleate, hexane, heptanes, nonane, decane, dodecane, R-limonene, D-limonene, neem oil, lavender oil, peppermint oil, anise oil, rosemary oil, sage oil, hibiscus oil, berries oil (any type), menthol, capsaicin, grape seed oil, pumpkin oil, hemp oil, toluene, methyl isobutyl ketone (MIBK), ethanol, methanol, isopropanol, glycerol, triacetin, ethyl laurate, ethyl hexyl laurate, ethyl hexyl oleate, benzyl alcohol, lauryl acetate, lauryl lactate, cetyl alcohol, petroleum ether, naphtha, ethyl acetate, butyl acetate, butyl butyrate, methyl tert-butyl ether (MBTE), and mixtures thereof. In some embodiments, the emollient comprises isopropyl myristate. In some embodiments, the emollient comprises oil, solvent, toluene, methyl isobutyl ketone (MIBK), heptanes, ethanol, methanol, isopropanol, isopropyl myristate (IPM), or any combination thereof. A particular emollient, or combination of emollients, may be selected for the methods of the disclosure because it is non-irritating to the skin. IPM is an example of such an emollient.

In some embodiments, the extraction of the cannabinoid or cannabinoid derivative (e.g., an acidic cannabinoid, acidic cannabinoid derivative, neutral cannabinoid, or neutral cannabinoid derivative) into the emollient phase is performed at a pH between about 4.0 and about 10.0 (i.e., the pH of the fermentation broth or washed fermentation broth is about 4.0 and about 10.0). In some embodiments, the extraction of the cannabinoid or cannabinoid derivative (e.g., an acidic cannabinoid, acidic cannabinoid derivative, neutral cannabinoid, or neutral cannabinoid derivative) into the emollient phase is performed at a pH between about 5.0 and about 9.0 (i.e., the pH of the fermentation broth or washed fermentation broth is about 5.0 and about 9.0). In some embodiments, the extraction of the cannabinoid or cannabinoid derivative (e.g., an acidic cannabinoid, acidic cannabinoid derivative, neutral cannabinoid, or neutral cannabinoid derivative) into the emollient phase is performed at a pH of about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, or about 10.0. In some embodiments, the extraction of the cannabinoid or cannabinoid derivative (e.g., an acidic cannabinoid, acidic cannabinoid derivative, neutral cannabinoid, or neutral cannabinoid derivative) into the emollient phase is performed at a pH between about 7.0 and about 8.0 (i.e., the pH of the fermentation broth or washed fermentation broth is about 7.0 and about 8.0). In some embodiments, the extraction of the cannabinoid or the cannabinoid derivative (e.g., an acidic cannabinoid, acidic cannabinoid derivative, neutral cannabinoid, or neutral cannabinoid derivative) into the emollient phase is performed at a pH of about 7.1, about 7.2, about 7.3, about 7.4, about 7.5, about 7.6, about 7.7, about 7.8, about 7.9, about 7.10, about 7.11, about 7.12, about 7.13, about 7.14, about 7.15, about 7.16, about 7.17, about 7.18, about 7.19, about 7.20, about 7.21, about 7.22, about 7.23, about 7.24, about 7.25, about 7.26, about 7.27, about 7.28, about 7.29, about 7.30, about 7.31, about 7.32, about 7.33, about 7.34, about 7.35, about 7.36, about 7.37, about 7.38, about 7.39, about 7.40, about 7.41, about 7.42, about 7.43, about 7.44, about 7.45, about 7.46, about 7.47, about 7.48, about 7.49, about 7.50, about 7.51, about 7.52, about 7.53, about 7.54, about 7.55, about 7.56, about 7.57, about 7.58, about 7.59, about 7.60, about 7.61, about 7.62, about 7.63, about 7.64, about 7.65, about 7.66, about 7.67, about 7.68, about 7.69, about 7.70, about 7.71, about 7.72, about 7.73, about 7.74, about 7.75, about 7.76, about 7.77, about 7.78, about 7.79, about 7.80, about 7.81, about 7.82, about 7.83, about 7.84, about 7.85, about 7.86, about 7.87, about 7.88, about 7.89, about 7.80, about 7.81, about 7.82, about 7.83, about 7.84, about 7.85, about 7.86, about 7.87, about 7.88, about 7.89, about 7.90, about 7.91, about 7.92, about 7.93, about 7.94, about 7.95, about 7.96, about 7.97, about 7.98, about 7.99, or about 8.0. In some embodiments, the extraction of the cannabinoid or the cannabinoid derivative (e.g., an acidic cannabinoid, acidic cannabinoid derivative, neutral cannabinoid, or neutral cannabinoid derivative) into the emollient phase is performed at a pH about 7.67 (i.e., the pH of the fermentation broth or washed fermentation broth is about 7.67). In some embodiments, the emollient phase comprises oil, solvent, toluene, methyl isobutyl ketone (MIBK), heptanes, ethanol, methanol, isopropanol, isopropyl myristate (IPM), or any combination thereof. In some embodiments, the emollient phase comprises IPM. In some embodiments, the cannabinoid or cannabinoid derivative is an acidic cannabinoid or acidic cannabinoid derivative. In some embodiments, the acidic cannabinoid or acidic cannabinoid derivative is cannabigerolic acid (CBGA), tetrahydrocannabinolic acid (THCA), or cannabidiolic acid (CBDA).

In some embodiments, the extraction of the cannabinoid or cannabinoid derivative (e.g., an acidic cannabinoid, acidic cannabinoid derivative, neutral cannabinoid, or neutral cannabinoid derivative) into the emollient phase is performed at a temperature between about 20° C. to about 50° C. In some embodiments, the extraction of the cannabinoid or cannabinoid derivative (e.g., an acidic cannabinoid, acidic cannabinoid derivative, neutral cannabinoid, or neutral cannabinoid derivative) into the emollient phase is performed at a temperature between about 30° C. to about 50° C. In some embodiments, the extraction of the cannabinoid or cannabinoid derivative (e.g., an acidic cannabinoid, acidic cannabinoid derivative, neutral cannabinoid, or neutral cannabinoid derivative) into the emollient phase is performed at a temperature of about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., about 40° C., about 41° C., about 42° C., about 43° C., about 44° C., about 45° C., about 46° C., about 47° C., about 48° C., about 49° C., or about 50° C. In some embodiments, the extraction of the cannabinoid or cannabinoid derivative (e.g., an acidic cannabinoid, acidic cannabinoid derivative, neutral cannabinoid, or neutral cannabinoid derivative) into the emollient phase is performed at a temperature of about 30° C.

In some aspects of the present disclosure, the cannabinoid or cannabinoid derivative (e.g., an acidic cannabinoid, acidic cannabinoid derivative, neutral cannabinoid, or neutral cannabinoid derivative) is extracted into an emollient mixture overlay. In some embodiments, the cannabinoid or cannabinoid derivative (e.g., an acidic cannabinoid, acidic cannabinoid derivative, neutral cannabinoid, or neutral cannabinoid derivative) is extracted into the emollient mixture that is at a ratio of about 1:1 v/v to about 1:20 v/v emollient phase: fermentation broth or washed fermentation broth. In some embodiments, the ratio of emollient phase to fermentation broth or washed fermentation broth is about 1:1 v/v, about 1:2 v/v, about 1:3 v/v, about 1:4 v/v, about 1:5 v/v, about 1:6 v/v, about 1:7 v/v, about 1:8 v/v, about 1:9 v/v, about 1:10 v/v, about 1:11 v/v, about 1:12 v/v, about 1:13 v/v, about 1:14 v/v, about 1:15 v/v, about 1:16 v/v, about 1:17 v/v, about 1:18 v/v, about 1:19 v/v, or about 1:20 v/v emollient phase: fermentation broth or washed fermentation broth. In some embodiments, the emollient phase comprises IPM. In some embodiments, the ratio of IPM to fermentation broth or washed fermentation broth is about 1:10.

In some aspects of the present disclosure, the cannabinoid or cannabinoid derivative (e.g., an acidic cannabinoid, acidic cannabinoid derivative, neutral cannabinoid, or neutral cannabinoid derivative) is extracted into an emollient phase overlay. In some embodiments, the cannabinoid or cannabinoid derivative (e.g., an acidic cannabinoid, acidic cannabinoid derivative, neutral cannabinoid, or neutral cannabinoid derivative) is extracted into an emollient phase that is about 1% to about 50% emollient in fermentation broth or washed fermentation broth. In some embodiments, the cannabinoid or cannabinoid derivative (e.g., an acidic cannabinoid, acidic cannabinoid derivative, neutral cannabinoid, or neutral cannabinoid derivative) is extracted into an emollient phase that is about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 40%, about 41%, about 42%, about 43%, about 44%, about 45%, about 46%, about 47%, about 48%, about 49%, or about 50% emollient in fermentation broth or washed fermentation broth. In some embodiments, the extraction of the cannabinoid or cannabinoid derivative (e.g., an acidic cannabinoid, acidic cannabinoid derivative, neutral cannabinoid, or neutral cannabinoid derivative) into the emollient phase is performed using about 10% v/v to about 20% v/v emollient. In some embodiments, the emollient is IPM. In some embodiments, the emollient phase is about 10% IPM in fermentation broth or washed fermentation broth.

In some embodiments, the cannabinoid or cannabinoid derivative (e.g., an acidic cannabinoid, acidic cannabinoid derivative, neutral cannabinoid, or neutral cannabinoid derivative) is extracted into an emollient phase for about 5 minutes to about 150 minutes. In some embodiments, the cannabinoid or cannabinoid derivative (e.g., an acidic cannabinoid, acidic cannabinoid derivative, neutral cannabinoid, or neutral cannabinoid derivative) is extracted into an emollient phase for about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 26 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, about 75 minutes, about 76 minutes, about 77 minutes, about 78 minutes, about 79 minutes, about 80 minutes, about 81 minutes, about 82 minutes, about 83 minutes, about 84 minutes, about 85 minutes, about 86 minutes, about 87 minutes, about 88 minutes, about 89 minutes, about 90 minutes, about 91 minutes, about 92 minutes, about 93 minutes, about 94 minutes, about 95 minutes, about 96 minutes, about 97 minutes, about 98 minutes, about 99 minutes, about 100 minutes, about 101 minutes, about 102 minutes, about 103 minutes, about 104 minutes, about 105 minutes, about 106 minutes, about 107 minutes, about 108 minutes, about 109 minutes, about 110 minutes, about 111 minutes, about 112 minutes, about 113 minutes, about 114 minutes, about 115 minutes, about 116 minutes, about 117 minutes, about 118 minutes, about 119 minutes, about 120 minutes, about 121 minutes, about 122 minutes, about 123 minutes, about 124 minutes, about 125 minutes, about 126 minutes, about 127 minutes, about 128 minutes, about 129 minutes, about 130 minutes, about 140 minutes, about 141 minutes, about 142 minutes, about 143 minutes, about 144 minutes, about 145 minutes, about 146 minutes, about 147 minutes, about 148 minutes, about 149 minutes, or about 150 minutes.

In some embodiments, the cannabinoid or cannabinoid derivative (e.g., an acidic cannabinoid, acidic cannabinoid derivative, neutral cannabinoid, or neutral cannabinoid derivative) is extracted into an emollient phase with agitation for about 5 minutes to about 150 minutes. In some embodiments, the cannabinoid or cannabinoid derivative (e.g., an acidic cannabinoid, acidic cannabinoid derivative, neutral cannabinoid, or neutral cannabinoid derivative) is extracted into an emollient phase with agitation for about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 26 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, about 75 minutes, about 76 minutes, about 77 minutes, about 78 minutes, about 79 minutes, about 80 minutes, about 81 minutes, about 82 minutes, about 83 minutes, about 84 minutes, about 85 minutes, about 86 minutes, about 87 minutes, about 88 minutes, about 89 minutes, about 90 minutes, about 91 minutes, about 92 minutes, about 93 minutes, about 94 minutes, about 95 minutes, about 96 minutes, about 97 minutes, about 98 minutes, about 99 minutes, about 100 minutes, about 101 minutes, about 102 minutes, about 103 minutes, about 104 minutes, about 105 minutes, about 106 minutes, about 107 minutes, about 108 minutes, about 109 minutes, about 110 minutes, about 111 minutes, about 112 minutes, about 113 minutes, about 114 minutes, about 115 minutes, about 116 minutes, about 117 minutes, about 118 minutes, about 119 minutes, about 120 minutes, about 121 minutes, about 122 minutes, about 123 minutes, about 124 minutes, about 125 minutes, about 126 minutes, about 127 minutes, about 128 minutes, about 129 minutes, about 130 minutes, about 140 minutes, about 141 minutes, about 142 minutes, about 143 minutes, about 144 minutes, about 145 minutes, about 146 minutes, about 147 minutes, about 148 minutes, about 149 minutes, or about 150 minutes. In some embodiments, the agitation is low agitation. In some embodiments, low agitation is achieved by centrifuging the two phases (e.g., the fermentation broth or washed fermentation broth and emollient). In some embodiments the two phases are centrifuged at about 500 to about 5000 rpm. In some embodiments, the two phases are centrifuged at about 500 rpm, about 600 rpm, about 700 rpm, about 800 rpm, about 900 rpm, about 1000 rpm, about 1100 rpm, about 1200 rpm, about 1300 rpm, about 1400 rpm, about 1500 rpm, about 1600 rpm, about 1700 rpm, about 1800 rpm, about 1900 rpm, about 2000 rpm, about 2100 rpm, about 2200 rpm, about 2300 rpm, about 2400 rpm, about 2500 rpm, about 2600 rpm, about 2700 rpm, about 2800 rpm, about 2900 rpm, about 3000 rpm, about 3100 rpm, about 3200 rpm, about 3300 rpm, about 3400 rpm, about 3500 rpm, about 3600 rpm, about 3700 rpm, about 3800 rpm, about 3900 rpm, about 4000 rpm, about 4100 rpm, about 4200 rpm, about 4300 rpm, about 4400 rpm, about 4500 rpm, about 4600 rpm, about 4700 rpm, about 4800 rpm, about 4900 rpm, or about 5000 rpm.

In some aspects of the present disclosure, the cannabinoid or cannabinoid derivative (e.g., an acidic cannabinoid, acidic cannabinoid derivative, neutral cannabinoid, or neutral cannabinoid derivative) is extracted into an emollient phase with low agitation for about 5 minutes to about 150 minutes. In some embodiments, the two phases are centrifuged at about 500 rpm, about 600 rpm, about 700 rpm, about 800 rpm, about 900 rpm, about 1000 rpm, about 1100 rpm, about 1200 rpm, about 1300 rpm, about 1400 rpm, about 1500 rpm, about 1600 rpm, about 1700 rpm, about 1800 rpm, about 1900 rpm, about 2000 rpm, about 2100 rpm, about 2200 rpm, about 2300 rpm, about 2400 rpm, about 2500 rpm, about 2600 rpm, about 2700 rpm, about 2800 rpm, about 2900 rpm, about 3000 rpm, about 3100 rpm, about 3200 rpm, about 3300 rpm, about 3400 rpm, about 3500 rpm, about 3600 rpm, about 3700 rpm, about 3800 rpm, about 3900 rpm, about 4000 rpm, about 4100 rpm, about 4200 rpm, about 4300 rpm, about 4400 rpm, about 4500 rpm, about 4600 rpm, about 4700 rpm, about 4800 rpm, about 4900 rpm, or about 5000 rpm for about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, about 30 minutes, about 31 minutes, about 32 minutes, about 33 minutes, about 34 minutes, about 35 minutes, about 26 minutes, about 37 minutes, about 38 minutes, about 39 minutes, about 40 minutes, about 41 minutes, about 42 minutes, about 43 minutes, about 44 minutes, about 45 minutes, about 46 minutes, about 47 minutes, about 48 minutes, about 49 minutes, about 50 minutes, about 51 minutes, about 52 minutes, about 53 minutes, about 54 minutes, about 55 minutes, about 56 minutes, about 57 minutes, about 58 minutes, about 59 minutes, about 60 minutes, about 61 minutes, about 62 minutes, about 63 minutes, about 64 minutes, about 65 minutes, about 66 minutes, about 67 minutes, about 68 minutes, about 69 minutes, about 70 minutes, about 71 minutes, about 72 minutes, about 73 minutes, about 74 minutes, about 75 minutes, about 76 minutes, about 77 minutes, about 78 minutes, about 79 minutes, about 80 minutes, about 81 minutes, about 82 minutes, about 83 minutes, about 84 minutes, about 85 minutes, about 86 minutes, about 87 minutes, about 88 minutes, about 89 minutes, about 90 minutes, about 91 minutes, about 92 minutes, about 93 minutes, about 94 minutes, about 95 minutes, about 96 minutes, about 97 minutes, about 98 minutes, about 99 minutes, about 100 minutes, about 101 minutes, about 102 minutes, about 103 minutes, about 104 minutes, about 105 minutes, about 106 minutes, about 107 minutes, about 108 minutes, about 109 minutes, about 110 minutes, about 111 minutes, about 112 minutes, about 113 minutes, about 114 minutes, about 115 minutes, about 116 minutes, about 117 minutes, about 118 minutes, about 119 minutes, about 120 minutes, about 121 minutes, about 122 minutes, about 123 minutes, about 124 minutes, about 125 minutes, about 126 minutes, about 127 minutes, about 128 minutes, about 129 minutes, about 130 minutes, about 140 minutes, about 141 minutes, about 142 minutes, about 143 minutes, about 144 minutes, about 145 minutes, about 146 minutes, about 147 minutes, about 148 minutes, about 149 minutes, or about 150 minutes. In some embodiments, the cannabinoid or cannabinoid derivative (e.g., an acidic cannabinoid, acidic cannabinoid derivative, neutral cannabinoid, or neutral cannabinoid derivative) is extracted into an emollient phase by adding 10% IPM to the fermentation broth or washed fermentation broth and centrifuging at 1000 rpm for 30 minutes at 30° C. In some embodiments, the cannabinoid or cannabinoid derivative is an acidic cannabinoid or acidic cannabinoid derivative. In some embodiments, the acidic cannabinoid or acidic cannabinoid derivative is cannabigerolic acid (CBGA), tetrahydrocannabinolic acid (THCA), or cannabidiolic acid (CBDA).

In some aspects of the present disclosure, after mixing the fermentation broth or washed fermentation broth with an emollient, the mixture is run through a three-phase centrifugation, separating the insoluble fraction (e.g., cells and cellular debris) from the emollient and aqueous phase and the emollient from the aqueous phase. The solid and aqueous fractions are discarded, and the emollient phase continues to the next step of the downstream processing disclosed herein.

In some embodiments, after extraction into an emollient phase, at least 50% of the cannabinoid or cannabinoid derivative (e.g., an acidic cannabinoid, acidic cannabinoid derivative, neutral cannabinoid, or neutral cannabinoid derivative) is retained in the emollient phase. In some embodiments, after extraction into an emollient phase, at least 70% of the cannabinoid or cannabinoid derivative (e.g., an acidic cannabinoid, acidic cannabinoid derivative, neutral cannabinoid, or neutral cannabinoid derivative) is retained in the emollient phase. In some embodiments, about 70% to about 100% of the cannabinoid or cannabinoid derivative (e.g., an acidic cannabinoid, acidic cannabinoid derivative, neutral cannabinoid, or neutral cannabinoid derivative) is retained in the emollient phase. In some embodiments, about 70%, about 71%, about 72%, about 73%, about 74%, about 75%, about, 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the cannabinoid or cannabinoid derivative (e.g., an acidic cannabinoid, acidic cannabinoid derivative, neutral cannabinoid, or neutral cannabinoid derivative) is retained in the emollient phase. In some embodiments, after extraction into an emollient phase, at least 90% of the cannabinoid or cannabinoid derivative (e.g., an acidic cannabinoid, acidic cannabinoid derivative, neutral cannabinoid, or neutral cannabinoid derivative) is retained in the emollient phase. In some embodiments, after extraction into an emollient phase, at least 95% of the cannabinoid or cannabinoid derivative (e.g., an acidic cannabinoid, acidic cannabinoid derivative, neutral cannabinoid, or neutral cannabinoid derivative) is retained in the emollient phase.

In some embodiments, extraction of the cannabinoid or cannabinoid derivative (e.g., an acidic cannabinoid, acidic cannabinoid derivative, neutral cannabinoid, or neutral cannabinoid derivative) into an emollient phase separates all or substantially all of the impurities and/or byproducts (e.g., olivetol, olivetolic acid, HTAL, PDAL, hexanoic acid) from the cannabinoid or cannabinoid derivative (e.g., an acidic cannabinoid, acidic cannabinoid derivative, neutral cannabinoid, or neutral cannabinoid derivative). In some embodiments, extraction of the cannabinoid or cannabinoid derivative (e.g., an acidic cannabinoid, acidic cannabinoid derivative, neutral cannabinoid, or neutral cannabinoid derivative) into an emollient phase removes at least 95% of the impurities and/or byproducts from the cannabinoid or cannabinoid derivative (e.g., an acidic cannabinoid, acidic cannabinoid derivative, neutral cannabinoid, or neutral cannabinoid derivative). In some embodiments, extraction of the cannabinoid or cannabinoid derivative (e.g., an acidic cannabinoid, acidic cannabinoid derivative, neutral cannabinoid, or neutral cannabinoid derivative) into an emollient phase removes about 50% to about 100% of the impurities and/or byproducts from the cannabinoid or cannabinoid derivative (e.g., an acidic cannabinoid, acidic cannabinoid derivative, neutral cannabinoid, or neutral cannabinoid derivative). In some embodiments, extraction of the cannabinoid or cannabinoid derivative (e.g., an acidic cannabinoid, acidic cannabinoid derivative, neutral cannabinoid, or neutral cannabinoid derivative) into an emollient phase removes about 50% to about 100% of the impurities and/or byproducts from the cannabinoid or cannabinoid derivative (e.g., an acidic cannabinoid, acidic cannabinoid derivative, neutral cannabinoid, or neutral cannabinoid derivative). In some embodiments, extraction of the cannabinoid or cannabinoid derivative (e.g., an acidic cannabinoid, acidic cannabinoid derivative, neutral cannabinoid, or neutral cannabinoid derivative) into an emollient phase removes about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the impurities and/or byproducts from the cannabinoid or cannabinoid derivative (e.g., an acidic cannabinoid, acidic cannabinoid derivative, neutral cannabinoid, or neutral cannabinoid derivative). In some embodiments, one or more impurities and/or byproducts present in the fermentation broth or washed fermentation broth are not significantly extracted into the emollient phase with the cannabinoid or cannabinoid derivative (e.g., an acidic cannabinoid, acidic cannabinoid derivative, neutral cannabinoid, or neutral cannabinoid derivative). In certain such embodiments, the one or more impurities and/or byproducts comprise olivetolic acid, PDAL, or HTAL, or a combination of any of the foregoing.

In some embodiments, extraction of the cannabinoid or cannabinoid derivative (e.g., an acidic cannabinoid, acidic cannabinoid derivative, neutral cannabinoid, or neutral cannabinoid derivative) into an emollient phase removes about 50% to 80% of the olivetol and substantially most (e.g., <5%) of the olivetolic acid, PDAL, and HTAL, while retaining about 95% of the cannabinoid or cannabinoid derivative (e.g., an acidic cannabinoid, acidic cannabinoid derivative, neutral cannabinoid, or neutral cannabinoid derivative) in the emollient phase. In some embodiments, the cannabinoid or cannabinoid derivative is an acidic cannabinoid or acidic cannabinoid derivative. In some embodiments, the acidic cannabinoid or acidic cannabinoid derivative is cannabigerolic acid (CBGA), tetrahydrocannabinolic acid (THCA), or cannabidiolic acid (CBDA).

Extraction of the Cannabinoid or Cannabinoid Derivative into an Aqueous Phase

In some embodiments, after the cannabinoid or cannabinoid derivative (e.g., an acidic cannabinoid, acidic cannabinoid derivative, neutral cannabinoid, or neutral cannabinoid derivative) is extracted into an emollient phase, it is then extracted into a fresh aqueous phase. To perform this extraction, water is added to the emollient phase and the pH adjusted. The change in pH alters the solubility of the cannabinoid or cannabinoid derivative (e.g., an acidic cannabinoid, acidic cannabinoid derivative, neutral cannabinoid, or neutral cannabinoid derivative) allowing it to migrate into the aqueous phase. After this extraction of the cannabinoid or cannabinoid derivative (e.g., an acidic cannabinoid, acidic cannabinoid derivative, neutral cannabinoid, or neutral cannabinoid derivative) into the aqueous phase, the phases are separated. In some embodiments, an optional chromatography step is performed to obtain a greater reduction in byproducts and/or impurities.

Water is added to the emollient phase to reach a ratio of emollient phase to aqueous phase of between about 1:1 v/v to about 1:20 v/v. In some embodiments the ratio of emollient phase to aqueous phase is about 1:1 v/v, about 1:2 v/v, about 1:3 v/v, about 1:4 v/v, about 1:5 v/v, about 1:6 v/v, about 1:7 v/v, about 1:8 v/v, about 1:9 v/v, about 1:10 v/v, about 1:11 v/v, about 1:12 v/v, about 1:13 v/v, about 1:14 v/v, about 1:15 v/v, about 1:16 v/v, about 1:17 v/v, about 1:18 v/v, about 1:19 v/v, or about 1:20 v/v emollient phase to aqueous phase. In some embodiments, the ratio of emollient phase to aqueous phase is between about 1:1 and 1:5 emollient phase:aqueous phase. In some embodiments, the ratio of emollient phase to aqueous phase is about 1:4 emollient phase:aqueous phase. In some embodiments, the emollient phase comprises IPM.

In some embodiments, the pH of the mixture to extract the cannabinoid or cannabinoid derivative (e.g., an acidic cannabinoid, acidic cannabinoid derivative, neutral cannabinoid, or neutral cannabinoid derivative) is adjusted by adding hydroxide ions. In some embodiments, the hydroxide solution is used at a concentration of about 10% to about 80% hydroxide. In some embodiments, the concentration of the hydroxide solution is about 10%, about 15%, about 20%, about 25%, about 30%, about 35%, about 40%, about 45%, about 50%, about 55%, about 60%, about 65%, about 70%, about 75%, or about 80%. In some embodiments, addition of potassium hydroxide or sodium hydroxide adjusts the pH of the extraction mixture. In some embodiments, the concentration potassium hydroxide or sodium hydroxide is about 25%. In some embodiments, 25% potassium hydroxide is used to adjust the pH of the extraction mixture. In some embodiments, 45% potassium hydroxide is used to adjust the pH of the extraction mixture.

In some embodiments, the extraction of the cannabinoid or cannabinoid derivative (e.g., an acidic cannabinoid, acidic cannabinoid derivative, neutral cannabinoid, or neutral cannabinoid derivative) into a fresh aqueous phase is performed at a pH between about 2.0 and about 14.0. In some embodiments, the extraction of the cannabinoid or cannabinoid derivative (e.g., an acidic cannabinoid, acidic cannabinoid derivative, neutral cannabinoid, or neutral cannabinoid derivative) into a fresh aqueous phase is performed at a pH between about 7.0 and about 11.0. In some embodiments, the extraction of the cannabinoid or cannabinoid derivative (e.g., an acidic cannabinoid, acidic cannabinoid derivative, neutral cannabinoid, or neutral cannabinoid derivative) into a fresh aqueous phase is performed at a pH of about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, about 12.0, about 12.1, about 12.2, about 12.3, about 12.4, about 12.5, about 12.6, about 12.7, about 12.8, about 12.9, about 13.0, about 13.1, about 13.2, about 13.3, about 13.4, about 13.5, about 13.6, about 13.7, about 13.8, about 13.9, or about 14.0. In some embodiments, the extraction of the cannabinoid or cannabinoid derivative (e.g., an acidic cannabinoid, acidic cannabinoid derivative, neutral cannabinoid, or neutral cannabinoid derivative) into a fresh aqueous phase is performed at a pH of about 11.5.

In some embodiments, the extraction of the cannabinoid or cannabinoid derivative (e.g., an acidic cannabinoid, acidic cannabinoid derivative, neutral cannabinoid, or neutral cannabinoid derivative) into a fresh aqueous phase is performed at a ratio of emollient phase to aqueous phase of between about 1:1 v/v to about 1:20 v/v at a pH between about 4.0 to about 12.0. In some embodiments the ratio of emollient phase to aqueous phase is about 1:1 v/v, about 1:2 v/v, about 1:3 v/v, about 1:4 v/v, about 1:5 v/v, about 1:6 v/v, about 1:7 v/v, about 1:8 v/v, about 1:9 v/v, about 1:10 v/v, about 1:11 v/v, about 1:12 v/v, about 1:13 v/v, about 1:14 v/v, about 1:15 v/v, about 1:16 v/v, about 1:17 v/v, about 1:18 v/v, about 1:19 v/v, or about 1:20 v/v emollient to water, at a pH of about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, or about 12.0.

In some embodiments, extraction of the cannabinoid or cannabinoid derivative (e.g., an acidic cannabinoid, acidic cannabinoid derivative, neutral cannabinoid, or neutral cannabinoid derivative) is performed by mild agitation which avoids emulsification of the emollient phase and the aqueous phase. In some embodiments, after mild agitation the phases are separated using a liquid:liquid separator to obtain an aqueous phase containing the cannabinoid or cannabinoid derivative (e.g., an acidic cannabinoid, acidic cannabinoid derivative, neutral cannabinoid, or neutral cannabinoid derivative) without residual emollient.

In some embodiments, the cannabinoid or cannabinoid derivative (e.g., an acidic cannabinoid, acidic cannabinoid derivative, neutral cannabinoid, or neutral cannabinoid derivative) is extracted into a fresh aqueous phase over about zero to about 24 hours. In some embodiments, the cannabinoid or cannabinoid derivative (e.g., an acidic cannabinoid, acidic cannabinoid derivative, neutral cannabinoid, or neutral cannabinoid derivative) is extracted into a fresh aqueous phase for about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5 about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, or about 24 hours.

In some embodiments, the cannabinoid or cannabinoid derivative (e.g., an acidic cannabinoid, acidic cannabinoid derivative, neutral cannabinoid, or neutral cannabinoid derivative) is present in the emollient phase at a concentration greater than about 0.1 mg/L. In some embodiments, the cannabinoid or cannabinoid derivative (e.g., an acidic cannabinoid, acidic cannabinoid derivative, neutral cannabinoid, or neutral cannabinoid derivative) is present in the emollient phase at a concentration of about 0.1 mg/L to about 500 g/L. In some embodiments, the cannabinoid or cannabinoid derivative (e.g., an acidic cannabinoid, acidic cannabinoid derivative, neutral cannabinoid, or neutral cannabinoid derivative) is present in the emollient phase at a concentration greater than about 500 g/L. In some embodiments, the cannabinoid or cannabinoid derivative (e.g., an acidic cannabinoid, acidic cannabinoid derivative, neutral cannabinoid, or neutral cannabinoid derivative) is present in the emollient phase at a concentration of about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.1 mg/L, about 1.2 mg/L, about 1.3 mg/L, about 1.4 mg/L, about 1.5 mg/L, about 1.6 mg/L, about 1.7 mg/L, about 1.8 mg/L, about 1.9 mg/L, about 2.0 mg/L, about 3 mg/L, about 4.0 mg/L, about 5.0 mg/L, about 6.0 mg/L, about 7.0 mg/L, about 8.0 mg/L, about 9.0 mg/L, about 10 mg/L, about 11 mg/L, about 12 mg/L, about 13 mg/L, about 14 mg/L, about 15 mg/L, about 16 mg/L, about 17 mg/L, about 18 mg/L, about 19 mg/L, about 20 mg/L, about 21 mg/L, about 22 mg/L, about 23 mg/L, about 24 mg/L, about 25 mg/L, about 26 mg/L, about 27 mg/L, about 28 mg/L, about 29 mg/L, about 30 mg/L, about 31 mg/L, about 32 mg/L, about 33 mg/L, about 34 mg/L, about 35 mg/L, about 36 mg/L, about 37 mg/L, about 38 mg/L, about 39 mg/L, about 40 mg/L, about 41 mg/L, about 42 mg/L, about 43 mg/L, about 44 mg/L, about 45 mg/L, about 46 mg/L, about 47 mg/L, about 48 mg/L, about 49 mg/L, about 50 mg/L, about 51 mg/L, about 52 mg/L, about 53 mg/L, about 54 mg/L, about 55 mg/L, about 56 mg/L, about 57 mg/L, about 58 mg/L, about 59 mg/L, about 60 mg/L, about 61 mg/L, about 62 mg/L, about 63 mg/L, about 64 mg/L, about 65 mg/L, about 66 mg/L, about 67 mg/L, about 68 mg/L, about 69 mg/L, about 70 mg/L, about 71 mg/L, about 72 mg/L, about 73 mg/L, about 74 mg/L, about 75 mg/L, about 76 mg/L, about 77 mg/L, about 78 mg/L, about 79 mg/L, about 80 mg/L, about 81 mg/L, about 82 mg/L, about 83 mg/L, about 84 mg/L, about 85 mg/L, about 86 mg/L, about 87 mg/L, about 88 mg/L, about 89 mg/L, about 90 mg/L, about 91 mg/L, about 92 mg/L, about 93 mg/L, about 94 mg/L, about 95 mg/L, about 96 mg/L, about 97 mg/L, about 98 mg/L, about 99 mg/L, about 100 mg/L, about 200 mg/L, about 300 mg/L, about 400 mg/L, about 500 mg/L, about 600 mg/L, about 700 mg/L, about 800 mg/L, about 900 mg/L, about lg/L, about 5 g/L, about, 10 g/L, about 15 g/L, about 20 g/L, about 30 g/L, about 40 g/L, about 50 g/L, about 60 g/L, about 70 g/L, about 80 g/L, about 90 g/L, about 100 g/L, about 200 g/L, about 300 g/L, about 400 g/L, about 500 g/L, or greater than about 500 g/L.

In some embodiments, the cannabinoid or cannabinoid derivative (e.g., an acidic cannabinoid, acidic cannabinoid derivative, neutral cannabinoid, or neutral cannabinoid derivative) is present in the emollient phase at a concentration of about 0.1 mg/L to about 500 g/L at about zero to about 24 hours. In some embodiments, the cannabinoid or cannabinoid derivative (e.g., an acidic cannabinoid, acidic cannabinoid derivative, neutral cannabinoid, or neutral cannabinoid derivative) is present in the emollient phase at a concentration of greater than about 500 g/L at about zero to about 24 hours. In some embodiments, the cannabinoid or cannabinoid derivative (e.g., an acidic cannabinoid, acidic cannabinoid derivative, neutral cannabinoid, or neutral cannabinoid derivative) is present in the emollient phase at a concentration of about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.1 mg/L, about 1.2 mg/L, about 1.3 mg/L, about 1.4 mg/L, about 1.5 mg/L, about 1.6 mg/L, about 1.7 mg/L, about 1.8 mg/L, about 1.9 mg/L, about 2.0 mg/L, about 3 mg/L, about 4.0 mg/L, about 5.0 mg/L, about 6.0 mg/L, about 7.0 mg/L, about 8.0 mg/L, about 9.0 mg/L, about 10 mg/L, about 11 mg/L, about 12 mg/L, about 13 mg/L, about 14 mg/L, about 15 mg/L, about 16 mg/L, about 17 mg/L, about 18 mg/L, about 19 mg/L, about 20 mg/L, about 21 mg/L, about 22 mg/L, about 23 mg/L, about 24 mg/L, about 25 mg/L, about 26 mg/L, about 27 mg/L, about 28 mg/L, about 29 mg/L, about 30 mg/L, about 31 mg/L, about 32 mg/L, about 33 mg/L, about 34 mg/L, about 35 mg/L, about 36 mg/L, about 37 mg/L, about 38 mg/L, about 39 mg/L, about 40 mg/L, about 41 mg/L, about 42 mg/L, about 43 mg/L, about 44 mg/L, about 45 mg/L, about 46 mg/L, about 47 mg/L, about 48 mg/L, about 49 mg/L, about 50 mg/L, about 51 mg/L, about 52 mg/L, about 53 mg/L, about 54 mg/L, about 55 mg/L, about 56 mg/L, about 57 mg/L, about 58 mg/L, about 59 mg/L, about 60 mg/L, about 61 mg/L, about 62 mg/L, about 63 mg/L, about 64 mg/L, about 65 mg/L, about 66 mg/L, about 67 mg/L, about 68 mg/L, about 69 mg/L, about 70 mg/L, about 71 mg/L, about 72 mg/L, about 73 mg/L, about 74 mg/L, about 75 mg/L, about 76 mg/L, about 77 mg/L, about 78 mg/L, about 79 mg/L, about 80 mg/L, about 81 mg/L, about 82 mg/L, about 83 mg/L, about 84 mg/L, about 85 mg/L, about 86 mg/L, about 87 mg/L, about 88 mg/L, about 89 mg/L, about 90 mg/L, about 91 mg/L, about 92 mg/L, about 93 mg/L, about 94 mg/L, about 95 mg/L, about 96 mg/L, about 97 mg/L, about 98 mg/L, about 99 mg/L, about 100 mg/L, about 200 mg/L, about 300 mg/L, about 400 mg/L, about 500 mg/L, about 600 mg/L, about 700 mg/L, about 800 mg/L, about 900 mg/L, about lg/L, about 5 g/L, about, 10 g/L, about 15 g/L, about 20 g/L, about 30 g/L, about 40 g/L, about 50 g/L, about 60 g/L, about 70 g/L, about 80 g/L, about 90 g/L, about 100 g/L, about 200 g/L, about 300 g/L, about 400 g/L, about 500 g/L, or greater than about 500 g/L at about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5 about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, or about 24 hours.

In some embodiments, extraction of the cannabinoid or cannabinoid derivative (e.g., an acidic cannabinoid, acidic cannabinoid derivative, neutral cannabinoid, or neutral cannabinoid derivative) into a fresh aqueous phase separates all or substantially all of the byproducts and/or impurities (e.g., olivetol, olivetolic acid, HTAL, PDAL, hexanoic acid) from the cannabinoid or cannabinoid derivative (e.g., an acidic cannabinoid, acidic cannabinoid derivative, neutral cannabinoid, or neutral cannabinoid derivative). In some embodiments, extraction of the cannabinoid or cannabinoid derivative (e.g., an acidic cannabinoid, acidic cannabinoid derivative, neutral cannabinoid, or neutral cannabinoid derivative) into a fresh aqueous phase removes at least 95% of the byproducts and/or impurities from the cannabinoid or cannabinoid derivative (e.g., an acidic cannabinoid, acidic cannabinoid derivative, neutral cannabinoid, or neutral cannabinoid derivative). In some embodiments, extraction of the cannabinoid or cannabinoid derivative (e.g., an acidic cannabinoid, acidic cannabinoid derivative, neutral cannabinoid, or neutral cannabinoid derivative) into a fresh aqueous phase removes about 50% to about 100% of the byproducts and/or impurities from the cannabinoid or cannabinoid derivative (e.g., an acidic cannabinoid, acidic cannabinoid derivative, neutral cannabinoid, or neutral cannabinoid derivative). In some embodiments, extraction of the cannabinoid or cannabinoid derivative (e.g., an acidic cannabinoid, acidic cannabinoid derivative, neutral cannabinoid, or neutral cannabinoid derivative) into a fresh aqueous phase removes about 50% to about 100% of the byproducts and/or impurities from the cannabinoid or cannabinoid derivative (e.g., an acidic cannabinoid, acidic cannabinoid derivative, neutral cannabinoid, or neutral cannabinoid derivative). In some embodiments, extraction of the cannabinoid or cannabinoid derivative (e.g., an acidic cannabinoid, acidic cannabinoid derivative, neutral cannabinoid, or neutral cannabinoid derivative) into a fresh aqueous phase removes about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the byproducts and/or impurities from the cannabinoid or cannabinoid derivative (e.g., an acidic cannabinoid, acidic cannabinoid derivative, neutral cannabinoid, or neutral cannabinoid derivative).

In some embodiments, extraction of the cannabinoid or cannabinoid derivative (e.g., an acidic cannabinoid, acidic cannabinoid derivative, neutral cannabinoid, or neutral cannabinoid derivative) into a fresh aqueous phase removes about 50% to about 80% of the olivetol and substantially most of the olivetolic acid, PDAL, and HTAL, while retaining about 95% of the cannabinoid or cannabinoid derivative (e.g., an acidic cannabinoid, acidic cannabinoid derivative, neutral cannabinoid, or neutral cannabinoid derivative) in the aqueous phase. In some embodiments, the cannabinoid or cannabinoid derivative is an acidic cannabinoid or acidic cannabinoid derivative. In some embodiments, the acidic cannabinoid or acidic cannabinoid derivative is cannabigerolic acid (CBGA), tetrahydrocannabinolic acid (THCA), or cannabidiolic acid (CBDA).

In some embodiments, at least about 50% of the cannabinoid or cannabinoid derivative (e.g., an acidic cannabinoid, acidic cannabinoid derivative, neutral cannabinoid, or neutral cannabinoid derivative) present in the emollient phase is extracted into the aqueous phase. In some embodiments, at least about 90% of the cannabinoid or cannabinoid derivative (e.g., an acidic cannabinoid, acidic cannabinoid derivative, neutral cannabinoid, or neutral cannabinoid derivative) present in the emollient phase is extracted into the aqueous phase.

In some embodiments, extraction of the cannabinoid or cannabinoid derivative (e.g., an acidic cannabinoid, acidic cannabinoid derivative, neutral cannabinoid, or neutral cannabinoid derivative) into the aqueous phase is performed by vigorous mixing resulting in hydrolysis of the emollient phase. In some embodiments, after vigorous mixing, the phases are separated using centrifugation followed by pumping out the aqueous phase leaving behind the hydrolyzed emollient layers.

In some aspects, the aqueous phase containing the cannabinoid or cannabinoid derivative (e.g., an acidic cannabinoid, acidic cannabinoid derivative, neutral cannabinoid, or neutral cannabinoid derivative) may optionally be subjected to chromatography. This step may reduce the amount of impurities and/or byproducts while recovering the cannabinoid or cannabinoid derivative (e.g., an acidic cannabinoid, acidic cannabinoid derivative, neutral cannabinoid, or neutral cannabinoid derivative) in the aqueous phase. Any appropriate chromatography method may be used, including but not limited to, reverse phase chromatography high performance liquid chromatography, ion exchange chromatography, gel electrophoresis, flash chromatography using silica gel (C8 or C18), preparative HPLC using silica gel column (C8 or C18), supercritical CO₂ chromatography, and affinity chromatography. In some embodiments, affinity chromatography is used.

In some aspects, the emollient phase containing the cannabinoid or cannabinoid derivative (e.g., an acidic cannabinoid, acidic cannabinoid derivative, neutral cannabinoid, or neutral cannabinoid derivative) may optionally be subjected to washing before extraction into a fresh aqueous phase. This optional wash may be performed at a pH between about 4.0 to about 12.0. In some embodiments, the emollient phase is washed at a pH of about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, or about 12.0.

In some embodiments, the optional wash may be repeated. In some embodiments, the optional wash is repeated 2-10 times. In some embodiments, the optional wash is repeated 2, 3, 4, 5, 6, 7, 8, 9, or 10 times. In some embodiments, the duration of the one or more optional washes is between about 5 minutes and about 2 hours. In some embodiments, the duration of the one or more optional washes is about 5 minutes, about 10 minutes, about 15 minutes, about 20 minutes, about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 115 minutes, or about 120 minutes. In some embodiments, the duration of the one or more optional washes is about 20 minutes. If more than one optional wash is performed, each wash may be the same duration. In some embodiments, the one or more optional washes have different durations.

In some embodiments, the one or more optional washes removes additional impurities and/or byproducts without losing significant amounts of the cannabinoid or cannabinoid derivative (e.g., an acidic cannabinoid, acidic cannabinoid derivative, neutral cannabinoid, or neutral cannabinoid derivative). In some embodiments, the cannabinoid or cannabinoid derivative is an acidic cannabinoid or cannabinoid derivative. In some embodiments, the acidic cannabinoid or acidic cannabinoid derivative is cannabigerolic acid (CBGA), tetrahydrocannabinolic acid (THCA), or cannabidiolic acid (CBDA).

### Decarboxylation of Acidic Cannabinoids or Acidic Cannabinoid Derivatives

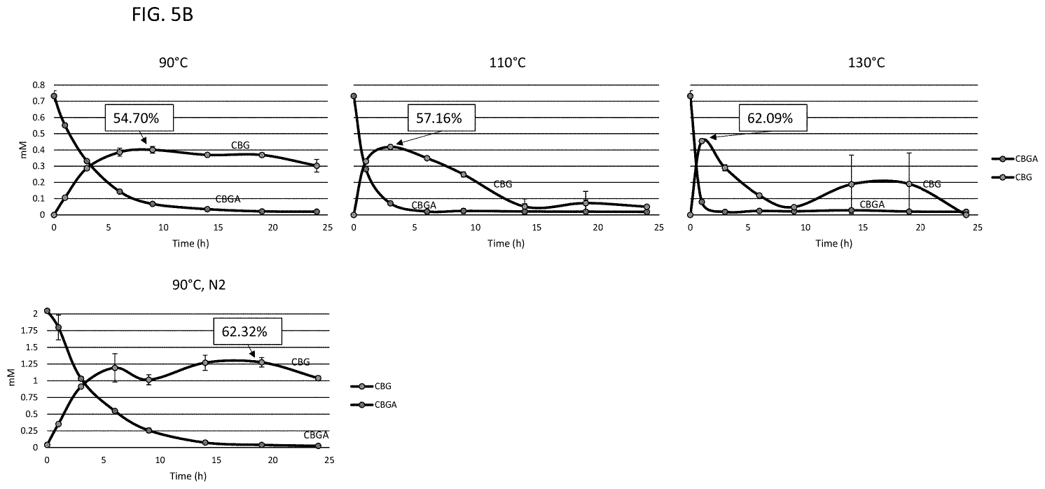

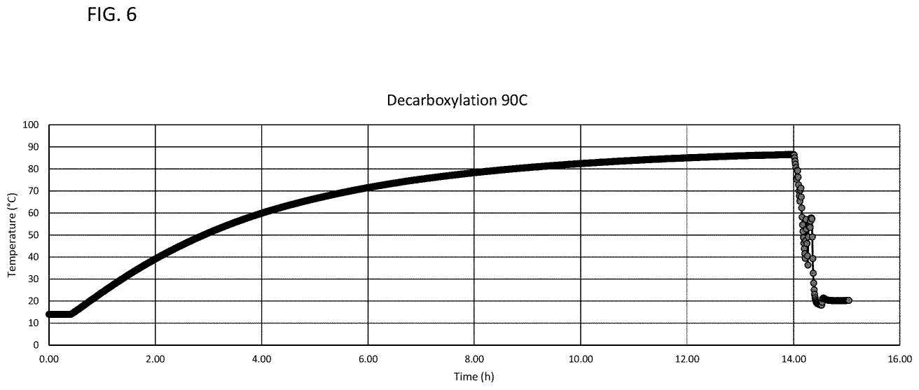

In some embodiments wherein the cannabinoids or cannabinoid derivatives are acidic cannabinoids or acidic cannabinoid derivatives, after extraction of acidic cannabinoids or acidic cannabinoid derivatives into an emollient phase, the emollient phase is subjected to decarboxylation. See FIGS. 5-6. In some embodiments, after extraction of acidic cannabinoids or acidic cannabinoid derivatives from an emollient phase into a fresh aqueous phase, the fresh aqueous phase is subjected to decarboxylation.

Decarboxylation is a chemical reaction that removes a carboxyl-group and releases carbon dioxide thereby creating a neutral cannabinoid or neutral cannabinoid derivative from an acidic cannabinoid or acidic cannabinoid derivative. In some embodiments, the rate of decarboxylation is dependent on heat. In some embodiments, the rate of decarboxylation is dependent on pH. In some embodiments, the rate of decarboxylation is dependent on heat and pH.

In some embodiments, decarboxylation of acidic cannabinoids or acidic cannabinoid derivatives is performed at a temperature between about 50° C. and about 140° C. In some embodiments, decarboxylation of acidic cannabinoids or acidic cannabinoid derivatives is performed at a temperature of about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., about 105° C., about 106° C., about 107° C., about 108° C., about 109° C., about 110° C., about 111° C., about 112° C., about 113° C., about 114° C., about 115° C., about 116° C., about 117° C., about 118° C., about 119° C., about 120° C., about 121° C., about 122° C., about 123° C., about 124° C., about 125° C., about 126° C., about 127° C., about 128° C., about 129° C., about 130° C., about 131° C., about 132° C., about 133° C., about 143° C., about 135° C., about 136° C., about 137° C., about 138° C., about 139° C., or about 140° C. In some embodiments, decarboxylation of acidic cannabinoids or acidic cannabinoid derivatives is performed at temperatures greater than about 70° C. In some embodiments, decarboxylation of acidic cannabinoids or acidic cannabinoid derivatives is performed at a temperature between about 80° C. and about 140° C. In some embodiments, decarboxylation of acidic cannabinoids or acidic cannabinoid derivatives is performed at a temperature between about 90° C. and about 130° C.

In some embodiments, decarboxylation of acidic cannabinoids or acidic cannabinoid derivatives is performed for about zero to about 24 hours. In some embodiments, decarboxylation of acidic cannabinoids or acidic cannabinoid derivatives is performed for about 0, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5 about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, or about 24 hours. In some embodiments, decarboxylation of acidic cannabinoids or acidic cannabinoid derivatives is performed for about 20 minutes. In some embodiments, decarboxylation of acidic cannabinoids or acidic cannabinoid derivatives is performed for about 14 hours. In some embodiments, decarboxylation of acidic cannabinoids or acidic cannabinoid derivatives is performed for about 5 minutes to about 20 hours.

In some embodiments, decarboxylation of acidic cannabinoids or acidic cannabinoid derivatives is performed at a temperature between about 50° C. and about 140° C. for about zero to about 24 hours. In some embodiments, decarboxylation of acidic cannabinoids or acidic cannabinoid derivatives is performed at a temperature of about 50° C., about 51° C., about 52° C., about 53° C., about 54° C., about 55° C., about 56° C., about 57° C., about 58° C., about 59° C., about 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., about 80° C., about 81° C., about 82° C., about 83° C., about 84° C., about 85° C., about 86° C., about 87° C., about 88° C., about 89° C., about 90° C., about 91° C., about 92° C., about 93° C., about 94° C., about 95° C., about 96° C., about 97° C., about 98° C., about 99° C., about 100° C., about 101° C., about 102° C., about 103° C., about 104° C., about 105° C., about 106° C., about 107° C., about 108° C., about 109° C., about 110° C., about 111° C., about 112° C., about 113° C., about 114° C., about 115° C., about 116° C., about 117° C., about 118° C., about 119° C., about 120° C., about 121° C., about 122° C., about 123° C., about 124° C., about 125° C., about 126° C., about 127° C., about 128° C., about 129° C., about 130° C., about 131° C., about 132° C., about 133° C., about 143° C., about 135° C., about 136° C., about 137° C., about 138° C., about 139° C., or about 140° C. for about 0, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5 about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, or about 24 hours. In some embodiments, decarboxylation of acidic cannabinoids or acidic cannabinoid derivatives is performed at about 90° C. for about 14 hours.

In some embodiments, decarboxylation of acidic cannabinoids or acidic cannabinoid derivatives is performed at fluctuating temperatures. In some embodiments, decarboxylation of acidic cannabinoids or acidic cannabinoid derivatives is performed at increasing temperatures. In some embodiments, decarboxylation of acidic cannabinoids or acidic cannabinoid derivatives is performed by increasing the temperature from about 0° C. to about 130° C. In some embodiments, decarboxylation of acidic cannabinoids or acidic cannabinoid derivatives by increasing the temperature from about 10° C. to about 90° C. In some embodiments, decarboxylation of acidic cannabinoids or acidic cannabinoid derivatives is performed by increasing the temperature from about 0° C. to about 130° C. over about 24 hours. In some embodiments, decarboxylation of acidic cannabinoid or acidic cannabinoid derivative by increasing the temperature from about 10° C. to about 90° C. over about 14 hours.

In some embodiments, decarboxylation of acidic cannabinoid or acidic cannabinoid derivative is performed at a pH between about 4.0 and about 12.0. In some embodiments, decarboxylation of acidic cannabinoids or acidic cannabinoid derivatives is performed at a pH between about 7.0 and about 11.0. In some embodiments, decarboxylation of acidic cannabinoids or acidic cannabinoid derivatives is performed at a pH of about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, or about 12.0.

In some embodiments, decarboxylation of acidic cannabinoids or acidic cannabinoid derivatives is performed at about 110° C. for about 0.5 hours (i.e. 30 minutes). In some embodiments, decarboxylation of acidic cannabinoids or acidic cannabinoid derivatives is performed at about 90° C. for about 14 hours in an aqueous environment. In some embodiments, the aqueous environment is covered with an overlay to prevent oxidation and unwanted chemical reactions of oxygen with other compounds present in the aqueous phase. In some embodiments, the overlay is an N₂ overlay.

In some embodiments, the yield of neutral cannabinoids or neutral cannabinoid derivatives afforded by decarboxylation of acidic cannabinoids or acidic cannabinoid derivatives is at between about 20% and 100%. In some embodiments, the yield of neutral cannabinoids or neutral cannabinoid derivatives afforded by decarboxylation of acidic cannabinoids or acidic cannabinoid derivatives is at least about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%. In some embodiments, the yield of the neutral cannabinoid or neutral cannabinoid derivative afforded by decarboxylation of the acidic cannabinoid or acidic cannabinoid derivative is at least about 50%. In some embodiments, the yield of the neutral cannabinoid or neutral cannabinoid derivative afforded by decarboxylation of the acidic cannabinoid or acidic cannabinoid derivative is at least about 60%. In some embodiments, the yield of the neutral cannabinoid or neutral cannabinoid derivative afforded by decarboxylation of the acidic cannabinoid or acidic cannabinoid derivative is at between about 60% to about 100%.

In some embodiments, the molar yield of the neutral cannabinoid or neutral cannabinoid derivative afforded by decarboxylation is determined based on the total acidic cannabinoid or acidic cannabinoid derivative present in the emollient phase before decarboxylation. In some embodiments, the yield of the neutral cannabinoid or neutral cannabinoid derivative afforded by decarboxylation of the acidic cannabinoid or acidic cannabinoid derivative is at least about a 30% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 40% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 50% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 60% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 70% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 80% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 85% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 90% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 91% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 92% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 93% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 94% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 95% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 96% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 97% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 98% molar yield based on the total acidic cannabinoid or acidic cannabinoid derivative subjected to decarboxylation. In some embodiments, the yield of the neutral cannabinoid or neutral cannabinoid derivative afforded by decarboxylation of the acidic cannabinoid or acidic cannabinoid derivative is at least about a 99% molar yield based on the total acidic cannabinoid or acidic cannabinoid derivative subjected to decarboxylation.

In some embodiments, the neutral cannabinoid or neutral cannabinoid derivative afforded by decarboxylation is present at a concentration greater than about 0.1 mM. In some embodiments, the neutral cannabinoid or neutral cannabinoid derivative afforded by decarboxylation is present at a concentration of about 0.1 mM to about 1.0 M. In some embodiments, the neutral cannabinoid or neutral cannabinoid derivative afforded by decarboxylation is present at a concentration greater than about 1.0 M. In some embodiments the neutral cannabinoid or neutral cannabinoid derivative afforded by decarboxylation is present at a concentration of about 0.1 mM, about 0.2 mM, about 0.3 mM, about 0.4 mM, about 0.5 mM, about 0.6 mM, about 0.7 mM, about 0.8 mM, about 0.9 mM, about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, about 2.0 mM, about 2.5 mM, about 3.0 mM, about 3.5 mM, about 4.0 mM, about 4.5 mM, about 5.0 mM, about 5.5 mM, about 6.0 mM, about 6.5 mM, about 7.0 mM, about 7.5 mM, about 8.0 mM, about 8.5 mM, about 9.0 mM, about 9.5 mM, about 10 mM, about 20 mM, about 30 mM, about 40 mM, about 50 mM, about 60 mM, about 70 mM, about 80 mM, about 90 mM, about 100 mM, about 150 mM, about 200 mM, about 250 mM, about 300 mM, about 350 mM, about 400 mM, about 450 mM, about 500 mM, about 550 mM, about 600 mM, about 650 mM, about 700 mM, about 750 mM, about 800 mM, about 850 mM, about 900 mM, about 950 mM, about 1.0 M, or greater than about 1.0 M.

In some embodiments, the neutral cannabinoid or neutral cannabinoid derivative afforded by decarboxylation is present at a concentration greater than about 0.1 mg/L. In some embodiments, the neutral cannabinoid or neutral cannabinoid derivative afforded by decarboxylation is present at a concentration of about 0.1 mg/L to about 500 g/L. In some embodiments, the neutral cannabinoid or neutral cannabinoid derivative afforded by decarboxylation is present at a concentration of greater than about 500 g/L. In some embodiments, the neutral cannabinoid or neutral cannabinoid derivative afforded by decarboxylation is present at a concentration of about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.1 mg/L, about 1.2 mg/L, about 1.3 mg/L, about 1.4 mg/L, about 1.5 mg/L, about 1.6 mg/L, about 1.7 mg/L, about 1.8 mg/L, about 1.9 mg/L, about 2.0 mg/L, about 3 mg/L, about 4.0 mg/L, about 5.0 mg/L, about 6.0 mg/L, about 7.0 mg/L, about 8.0 mg/L, about 9.0 mg/L, about 10 mg/L, about 11 mg/L, about 12 mg/L, about 13 mg/L, about 14 mg/L, about 15 mg/L, about 16 mg/L, about 17 mg/L, about 18 mg/L, about 19 mg/L, about 20 mg/L, about 21 mg/L, about 22 mg/L, about 23 mg/L, about 24 mg/L, about 25 mg/L, about 26 mg/L, about 27 mg/L, about 28 mg/L, about 29 mg/L, about 30 mg/L, about 31 mg/L, about 32 mg/L, about 33 mg/L, about 34 mg/L, about 35 mg/L, about 36 mg/L, about 37 mg/L, about 38 mg/L, about 39 mg/L, about 40 mg/L, about 41 mg/L, about 42 mg/L, about 43 mg/L, about 44 mg/L, about 45 mg/L, about 46 mg/L, about 47 mg/L, about 48 mg/L, about 49 mg/L, about 50 mg/L, about 51 mg/L, about 52 mg/L, about 53 mg/L, about 54 mg/L, about 55 mg/L, about 56 mg/L, about 57 mg/L, about 58 mg/L, about 59 mg/L, about 60 mg/L, about 61 mg/L, about 62 mg/L, about 63 mg/L, about 64 mg/L, about 65 mg/L, about 66 mg/L, about 67 mg/L, about 68 mg/L, about 69 mg/L, about 70 mg/L, about 71 mg/L, about 72 mg/L, about 73 mg/L, about 74 mg/L, about 75 mg/L, about 76 mg/L, about 77 mg/L, about 78 mg/L, about 79 mg/L, about 80 mg/L, about 81 mg/L, about 82 mg/L, about 83 mg/L, about 84 mg/L, about 85 mg/L, about 86 mg/L, about 87 mg/L, about 88 mg/L, about 89 mg/L, about 90 mg/L, about 91 mg/L, about 92 mg/L, about 93 mg/L, about 94 mg/L, about 95 mg/L, about 96 mg/L, about 97 mg/L, about 98 mg/L, about 99 mg/L, about 100 mg/L, about 200 mg/L, about 300 mg/L, about 400 mg/L, about 500 mg/L, about 600 mg/L, about 700 mg/L, about 800 mg/L, about 900 mg/L, about 1g/L, about 5 g/L, about 10 g/L, about 15 g/L, about 20 g/L, about 30 g/L, about 40 g/L, about 50 g/L, about 60 g/L, about 70 g/L, about 80 g/L, about 90 g/L, about 100 g/L, about 200 g/L, about 300 g/L, about 400 g/L, about 500 g/L, or greater than about 500 g/L.

In some embodiments, the neutral cannabinoid or neutral cannabinoid derivative afforded by decarboxylation is present at a concentration of about 0.1 mM to about 1.0 M at about zero to about 24 hours. In some embodiments, the neutral cannabinoid or neutral cannabinoid derivative afforded by decarboxylation is present at a concentration of greater than about 1.0 M at about zero to about 24 hours. In some embodiments the neutral cannabinoid or neutral cannabinoid derivative afforded by decarboxylation is present at a concentration of about 0.1 mM, about 0.2 mM, about 0.3 mM, about 0.4 mM, about 0.5 mM, about 0.6 mM, about 0.7 mM, about 0.8 mM, about 0.9 mM, about 1.0 mM, about 1.1 mM, about 1.2 mM, about 1.3 mM, about 1.4 mM, about 1.5 mM, about 1.6 mM, about 1.7 mM, about 1.8 mM, about 1.9 mM, about 2.0 mM, about 2.5 mM, about 3.0 mM, about 3.5 mM, about 4.0 mM, about 4.5 mM, about 5.0 mM, about 5.5 mM, about 6.0 mM, about 6.5 mM, about 7.0 mM, about 7.5 mM, about 8.0 mM, about 8.5 mM, about 9.0 mM, about 9.5 mM, about 10 mM, about 20 mM, about 30 mM, about 40 mM, about 50 mM, about 60 mM, about 70 mM, about 80 mM, about 90 mM, about 100 mM, about 150 mM, about 200 mM, about 250 mM, about 300 mM, about 350 mM, about 400 mM, about 450 mM, about 500 mM, about 550 mM, about 600 mM, about 650 mM, about 700 mM, about 750 mM, about 800 mM, about 850 mM, about 900 mM, about 950 mM, about 1.0 M, or greater than about 1.0 M at about 0, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5 about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, or about 24 hours.

In some embodiments, the neutral cannabinoid or neutral cannabinoid derivative afforded by decarboxylation is present at a concentration of about 0.1 mg/L to about 500 g/L at between about zero to about 24 hours. In some embodiments, the neutral cannabinoid or neutral cannabinoid derivative afforded by decarboxylation is present at a concentration of greater than about 500 g/L at between about zero to about 24 hours. In some embodiments, the neutral cannabinoid or neutral cannabinoid derivative afforded by decarboxylation is present at a concentration of about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.1 mg/L, about 1.2 mg/L, about 1.3 mg/L, about 1.4 mg/L, about 1.5 mg/L, about 1.6 mg/L, about 1.7 mg/L, about 1.8 mg/L, about 1.9 mg/L, about 2.0 mg/L, about 3 mg/L, about 4.0 mg/L, about 5.0 mg/L, about 6.0 mg/L, about 7.0 mg/L, about 8.0 mg/L, about 9.0 mg/L, about 10 mg/L, about 11 mg/L, about 12 mg/L, about 13 mg/L, about 14 mg/L, about 15 mg/L, about 16 mg/L, about 17 mg/L, about 18 mg/L, about 19 mg/L, about 20 mg/L, about 21 mg/L, about 22 mg/L, about 23 mg/L, about 24 mg/L, about 25 mg/L, about 26 mg/L, about 27 mg/L, about 28 mg/L, about 29 mg/L, about 30 mg/L, about 31 mg/L, about 32 mg/L, about 33 mg/L, about 34 mg/L, about 35 mg/L, about 36 mg/L, about 37 mg/L, about 38 mg/L, about 39 mg/L, about 40 mg/L, about 41 mg/L, about 42 mg/L, about 43 mg/L, about 44 mg/L, about 45 mg/L, about 46 mg/L, about 47 mg/L, about 48 mg/L, about 49 mg/L, about 50 mg/L, about 51 mg/L, about 52 mg/L, about 53 mg/L, about 54 mg/L, about 55 mg/L, about 56 mg/L, about 57 mg/L, about 58 mg/L, about 59 mg/L, about 60 mg/L, about 61 mg/L, about 62 mg/L, about 63 mg/L, about 64 mg/L, about 65 mg/L, about 66 mg/L, about 67 mg/L, about 68 mg/L, about 69 mg/L, about 70 mg/L, about 71 mg/L, about 72 mg/L, about 73 mg/L, about 74 mg/L, about 75 mg/L, about 76 mg/L, about 77 mg/L, about 78 mg/L, about 79 mg/L, about 80 mg/L, about 81 mg/L, about 82 mg/L, about 83 mg/L, about 84 mg/L, about 85 mg/L, about 86 mg/L, about 87 mg/L, about 88 mg/L, about 89 mg/L, about 90 mg/L, about 91 mg/L, about 92 mg/L, about 93 mg/L, about 94 mg/L, about 95 mg/L, about 96 mg/L, about 97 mg/L, about 98 mg/L, about 99 mg/L, about 100 mg/L, about 200 mg/L, about 300 mg/L, about 400 mg/L, about 500 mg/L, about 600 mg/L, about 700 mg/L, about 800 mg/L, about 900 mg/L, about lg/L, about 5 g/L, about, 10 g/L, about 15 g/L, about 20 g/L, about 30 g/L, about 40 g/L, about 50 g/L, about 60 g/L, about 70 g/L, about 80 g/L, about 90 g/L, about 100 g/L, about 200 g/L, about 300 g/L, about 400 g/L, about 500 g/L, or greater than about 500 g/L at about 0, about 0.1, about 0.2, about 0.3, about 0.4, about 0.5, about 0.6, about 0.7, about 0.8, about 0.9, about 1, about 1.1, about 1.2, about 1.3, about 1.4, about 1.5, about 1.6, about 1.7, about 1.8, about 1.9, about 2, about 2.5, about 3, about 3.5, about 4, about 4.5, about 5, about 5.5, about 6, about 6.5, about 7, about 7.5, about 8, about 8.5, about 9, about 9.5, about 10, about 10.5 about 11, about 11.5, about 12, about 12.5, about 13, about 13.5, about 14, about 14.5, about 15, about 15.5, about 16, about 16.5, about 17, about 17.5, about 18, about 18.5, about 19, about 19.5, about 20, about 20.5, about 21, about 21.5, about 22, about 22.5, about 23, about 23.5, or about 24 hours.

In some embodiments, after decarboxylation of the acidic cannabinoid or acidic cannabinoid derivative, all or substantially all of the byproducts and/or impurities (e.g., olivetol, olivetolic acid, HTAL, PDAL, hexanoic acid) are removed from the neutral cannabinoid or neutral cannabinoid derivative afforded by decarboxylation. In some embodiments, decarboxylation of the acidic cannabinoid or acidic cannabinoid derivative removes at least 95% of the byproducts and/or impurities from the neutral cannabinoid or neutral cannabinoid derivative afforded by decarboxylation. In some embodiments, decarboxylation of the acidic cannabinoid or acidic cannabinoid derivative removes about 50% to about 100% of the byproducts and/or impurities from the neutral cannabinoid or neutral cannabinoid derivative afforded by decarboxylation. In some embodiments, decarboxylation of the acidic cannabinoid or acidic cannabinoid derivative removes about 50% to about 100% of the byproducts and/or impurities from the neutral cannabinoid or neutral cannabinoid derivative afforded by decarboxylation. In some embodiments, decarboxylation of the acidic cannabinoid or acidic cannabinoid derivative removes about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the byproducts and/or impurities from the neutral cannabinoid or neutral cannabinoid derivative afforded by decarboxylation.

In some embodiments, decarboxylation of the acidic cannabinoid or acidic cannabinoid derivative removes about 50% to 80% of the olivetol and substantially most (e.g., <5%) of the olivetolic acid, PDAL, and HTAL, while retaining about 95% of the neutral cannabinoid or neutral cannabinoid derivative afforded by decarboxylation. In some embodiments, the neutral cannabinoid or neutral cannabinoid derivative is tetrahydrocannabinol (THC), cannabidiol (CBD), or cannabigerol (CBG). In some embodiments, the acidic cannabinoid or acidic cannabinoid derivative is cannabigerolic acid (CBGA), tetrahydrocannabinolic acid (THCA), or cannabidiolic acid (CBDA).

### Precipitation of the Cannabinoid or Cannabinoid Derivative

In some aspects, the methods of the present disclosure includes an optional step of precipitating the cannabinoid or cannabinoid derivative (e.g., acidic cannabinoid, neutral cannabinoid, acidic cannabinoid derivative, or neutral cannabinoid derivative) from an aqueous phase. Here, the precipitation of the cannabinoid or cannabinoid derivative (e.g., acidic cannabinoid, neutral cannabinoid, acidic cannabinoid derivative, or neutral cannabinoid derivative) may remove further impurities and/or byproducts.

In some embodiments, precipitation of the cannabinoid or cannabinoid derivative (e.g., acidic cannabinoid, neutral cannabinoid, acidic cannabinoid derivative, or neutral cannabinoid derivative) occurs at a pH between about 1.0 and about 12.0. In some embodiments, precipitation of the cannabinoid or cannabinoid derivative (e.g., acidic cannabinoid, neutral cannabinoid, acidic cannabinoid derivative, or neutral cannabinoid derivative) occurs at a pH of about 1.0, about 1.1, about 1.2, about 1.3, about 1.4, about 1.5, about 1.6, about 1.7, about 1.8, about 1.9, about 2.0, about 2.1, about 2.2., about 2.3, about 2.4, about 2.5, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, or about 12.0. In some embodiments, the cannabinoid or cannabinoid derivative (e.g., acidic cannabinoid, neutral cannabinoid, acidic cannabinoid derivative, or neutral cannabinoid derivative) precipitates at a pH below 2.0. In some embodiments, the cannabinoid or cannabinoid derivative (e.g., acidic cannabinoid, neutral cannabinoid, acidic cannabinoid derivative, or neutral cannabinoid derivative) precipitates at a pH below 12.0.

In some embodiments, precipitation of the cannabinoid or cannabinoid derivative (e.g., acidic cannabinoid, neutral cannabinoid, acidic cannabinoid derivative, or neutral cannabinoid derivative) retains at least 70% of the cannabinoid or cannabinoid derivative. In some embodiments, precipitation of the cannabinoid or cannabinoid derivative (e.g., acidic cannabinoid, neutral cannabinoid, acidic cannabinoid derivative, or neutral cannabinoid derivative) retains about 70% to about 100% of the cannabinoid or cannabinoid derivative. In some embodiments, precipitation of the cannabinoid or cannabinoid derivative (e.g., acidic cannabinoid, neutral cannabinoid, acidic cannabinoid derivative, or neutral cannabinoid derivative) retains about 70%, about 71%, about 72%, about 73%, about 74%, about 75%, about, 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the cannabinoid or cannabinoid derivative.

In some embodiments, the yield of the cannabinoid or cannabinoid derivative (e.g., acidic cannabinoid, neutral cannabinoid, acidic cannabinoid derivative, or neutral cannabinoid derivative) afforded by precipitation of the cannabinoid or cannabinoid derivative is at least about 50%. In some embodiments, the yield of the cannabinoid or cannabinoid derivative (e.g., acidic cannabinoid, neutral cannabinoid, acidic cannabinoid derivative, or neutral cannabinoid derivative) afforded by precipitation of the cannabinoid or cannabinoid derivative is at least about 60%. In some embodiments, the yield of the cannabinoid or cannabinoid derivative (e.g., acidic cannabinoid, neutral cannabinoid, acidic cannabinoid derivative, or neutral cannabinoid derivative) afforded by precipitation of the cannabinoid or cannabinoid derivative is between about 60% and about 100%. In some embodiments, the yield of the cannabinoid or cannabinoid derivative (e.g., acidic cannabinoid, neutral cannabinoid, acidic cannabinoid derivative, or neutral cannabinoid derivative) afforded by precipitation of the cannabinoid or cannabinoid derivative is about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the cannabinoid or cannabinoid derivative (e.g., acidic cannabinoid, neutral cannabinoid, acidic cannabinoid derivative, or neutral cannabinoid derivative) afforded by precipitation is present at a concentration greater than about 0.1 mg/L of precipitate in the aqueous phase. In some embodiments, the cannabinoid or cannabinoid derivative (e.g., acidic cannabinoid, neutral cannabinoid, acidic cannabinoid derivative, or neutral cannabinoid derivative) afforded by precipitation is present at a concentration of about 0.1 mg/L to about 500 g/L of precipitate in the aqueous phase. In some embodiments, the cannabinoid or cannabinoid derivative (e.g., acidic cannabinoid, neutral cannabinoid, acidic cannabinoid derivative, or neutral cannabinoid derivative) afforded by precipitation is present at a concentration of greater than about 500 g/L of precipitate in the aqueous phase. In some embodiments, the cannabinoid or cannabinoid derivative (e.g., acidic cannabinoid, neutral cannabinoid, acidic cannabinoid derivative, or neutral cannabinoid derivative) afforded by precipitation is present at a concentration of precipitate of about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.1 mg/L, about 1.2 mg/L, about 1.3 mg/L, about 1.4 mg/L, about 1.5 mg/L, about 1.6 mg/L, about 1.7 mg/L, about 1.8 mg/L, about 1.9 mg/L, about 2.0 mg/L, about 3 mg/L, about 4.0 mg/L, about 5.0 mg/L, about 6.0 mg/L, about 7.0 mg/L, about 8.0 mg/L, about 9.0 mg/L, about 10 mg/L, about 11 mg/L, about 12 mg/L, about 13 mg/L, about 14 mg/L, about 15 mg/L, about 16 mg/L, about 17 mg/L, about 18 mg/L, about 19 mg/L, about 20 mg/L, about 21 mg/L, about 22 mg/L, about 23 mg/L, about 24 mg/L, about 25 mg/L, about 26 mg/L, about 27 mg/L, about 28 mg/L, about 29 mg/L, about 30 mg/L, about 31 mg/L, about 32 mg/L, about 33 mg/L, about 34 mg/L, about 35 mg/L, about 36 mg/L, about 37 mg/L, about 38 mg/L, about 39 mg/L, about 40 mg/L, about 41 mg/L, about 42 mg/L, about 43 mg/L, about 44 mg/L, about 45 mg/L, about 46 mg/L, about 47 mg/L, about 48 mg/L, about 49 mg/L, about 50 mg/L, about 51 mg/L, about 52 mg/L, about 53 mg/L, about 54 mg/L, about 55 mg/L, about 56 mg/L, about 57 mg/L, about 58 mg/L, about 59 mg/L, about 60 mg/L, about 61 mg/L, about 62 mg/L, about 63 mg/L, about 64 mg/L, about 65 mg/L, about 66 mg/L, about 67 mg/L, about 68 mg/L, about 69 mg/L, about 70 mg/L, about 71 mg/L, about 72 mg/L, about 73 mg/L, about 74 mg/L, about 75 mg/L, about 76 mg/L, about 77 mg/L, about 78 mg/L, about 79 mg/L, about 80 mg/L, about 81 mg/L, about 82 mg/L, about 83 mg/L, about 84 mg/L, about 85 mg/L, about 86 mg/L, about 87 mg/L, about 88 mg/L, about 89 mg/L, about 90 mg/L, about 91 mg/L, about 92 mg/L, about 93 mg/L, about 94 mg/L, about 95 mg/L, about 96 mg/L, about 97 mg/L, about 98 mg/L, about 99 mg/L, about 100 mg/L, about 200 mg/L, about 300 mg/L, about 400 mg/L, about 500 mg/L, about 600 mg/L, about 700 mg/L, about 800 mg/L, about 900 mg/L, about lg/L, about 5 g/L, about, 10 g/L, about 15 g/L, about 20 g/L, about 30 g/L, about 40 g/L, about 50 g/L, about 60 g/L, about 70 g/L, about 80 g/L, about 90 g/L, about 100 g/L, about 200 g/L, about 300 g/L, about 400 g/L, about 500 g/L or greater than about 500 g/L in the aqueous phase.

In some embodiments, precipitation of the cannabinoid or cannabinoid derivative (e.g., acidic cannabinoid, neutral cannabinoid, acidic cannabinoid derivative, or neutral cannabinoid derivative) removes all or substantially all of the byproducts and/or impurities (e.g., olivetol, olivetolic acid, HTAL, PDAL, hexanoic acid) from the cannabinoid or cannabinoid derivative afforded by precipitation. In some embodiments, precipitation of the cannabinoid or cannabinoid derivative (e.g., acidic cannabinoid, neutral cannabinoid, acidic cannabinoid derivative, or neutral cannabinoid derivative) removes at least 95% of the byproducts and/or impurities from the cannabinoid or cannabinoid derivative afforded by precipitation. In some embodiments, precipitation of the cannabinoid or cannabinoid derivative (e.g., acidic cannabinoid, neutral cannabinoid, acidic cannabinoid derivative, or neutral cannabinoid derivative) removes about 50% to about 100% of the byproducts and/or impurities from the cannabinoid or cannabinoid derivative afforded by precipitation. In some embodiments, precipitation of the cannabinoid or cannabinoid derivative (e.g., acidic cannabinoid, neutral cannabinoid, acidic cannabinoid derivative, or neutral cannabinoid derivative) removes about 50% to about 100% of the byproducts and/or impurities from the cannabinoid or cannabinoid derivative afforded by precipitation. In some embodiments, precipitation of the cannabinoid or cannabinoid derivative (e.g., acidic cannabinoid, neutral cannabinoid, acidic cannabinoid derivative, or neutral cannabinoid derivative) removes about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the byproducts and/or impurities from the cannabinoid or cannabinoid derivative afforded by precipitation.

In some embodiments, precipitation of the cannabinoid or cannabinoid derivative (e.g., acidic cannabinoid, neutral cannabinoid, acidic cannabinoid derivative, or neutral cannabinoid derivative) removes about substantially most (e.g., <5%) of the olivetol, olivetolic acid, PDAL, and HTAL, while retaining about 95% of the cannabinoid or cannabinoid derivative. In some embodiments, the cannabinoid or cannabinoid derivative is a neutral cannabinoid or neutral cannabinoid derivative. In some embodiments, the neutral cannabinoid or neutral cannabinoid derivative is tetrahydrocannabinol (THC), cannabidiol (CBD), or cannabigerol (CBG). In some embodiments, the cannabinoid or cannabinoid derivative is an acidic cannabinoid or an acidic cannabinoid derivative. In some embodiments, the acidic cannabinoid or acidic cannabinoid derivative is cannabigerolic acid (CBGA), tetrahydrocannabinolic acid (THCA), or cannabidiolic acid (CBDA).

### Crystallization of the Cannabinoid or Cannabinoid Derivative and Recovery of the Cannabinoid or Cannabinoid Derivative Preparation

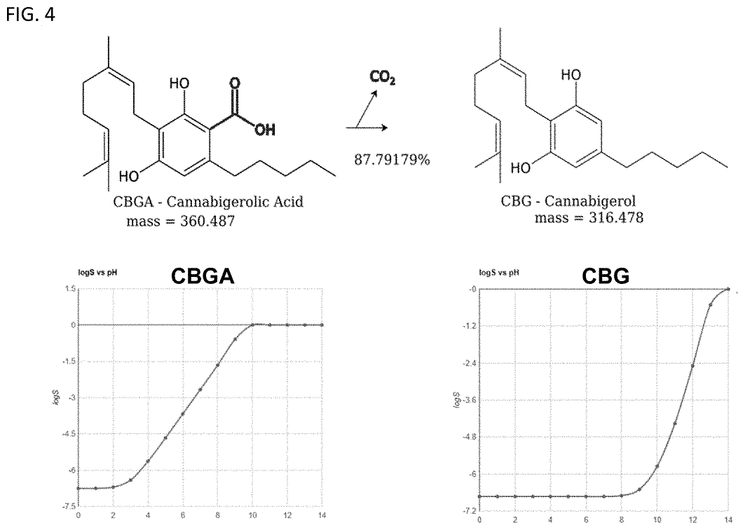

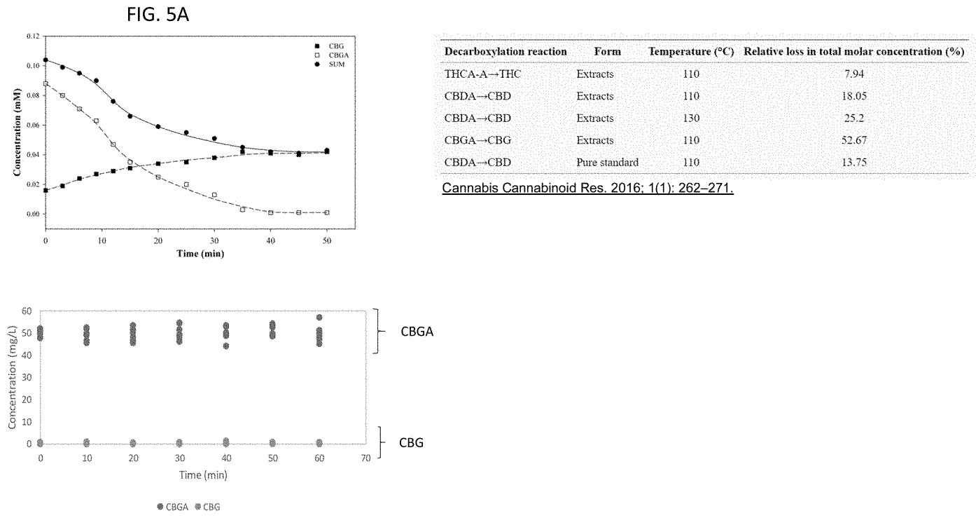

Crystallization of the cannabinoid or cannabinoid derivative (e.g., acidic cannabinoid, neutral cannabinoid, acidic cannabinoid derivative, or neutral cannabinoid derivative) in the aqueous phase occurs spontaneously at a critical concentration. For example, when the aqueous phase containing the neutral cannabinoid or neutral cannabinoid derivative afforded by decarboxylation cools slowly to about 25° C. or below, crystals form which can then be recovered. The pH of the solution determines which cannabinoid or cannabinoid derivative will crystalize; see for example the solubility profile of CBGA and CBG in FIG. 4. For example, where CGBA is insoluble in water at pH <2, CBG is insoluble at pH <10. The difference in solubility of the two compounds provides the basis of their purification in the downstream process disclosed herein. In some embodiments, the cannabinoid or cannabinoid derivative crystallized is a neutral cannabinoid or neutral cannabinoid derivative. In some embodiments, the cannabinoid or cannabinoid derivative crystallized is an acidic cannabinoid or an acidic cannabinoid derivative.

In some embodiments, crystallization of the cannabinoid or cannabinoid derivative is performed by slowly cooling an aqueous phase comprising the neutral cannabinoid or neutral cannabinoid derivative afforded by decarboxylation of the acidic cannabinoid or acidic cannabinoid derivative to about 25° C. or below. In some embodiments, crystallization of the cannabinoid or cannabinoid derivative is performed by slowly cooling an aqueous phase comprising the neutral cannabinoid or neutral cannabinoid derivative afforded by decarboxylation of the acidic cannabinoid or acidic cannabinoid derivative to a temperature between about −20° C. to about 40° C. In some embodiments, crystallization of the cannabinoid or cannabinoid derivative is performed by slowly cooling an aqueous phase comprising the neutral cannabinoid or neutral cannabinoid derivative afforded by decarboxylation of the acidic cannabinoid or acidic cannabinoid derivative to a temperature of about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

In some embodiments, crystallization of the cannabinoid or cannabinoid derivative (e.g., acidic cannabinoid, neutral cannabinoid, acidic cannabinoid derivative, or neutral cannabinoid derivative) is performed by slowly cooling an aqueous phase comprising the cannabinoid or cannabinoid derivative to about 25° C. or below. In some embodiments, crystallization of the cannabinoid or cannabinoid derivative (e.g., acidic cannabinoid, neutral cannabinoid, acidic cannabinoid derivative, or neutral cannabinoid derivative) is performed by slowly cooling an aqueous phase comprising the cannabinoid or cannabinoid derivative to a temperature between about −20° C. to about 40° C. In some embodiments, crystallization of the cannabinoid or cannabinoid derivative (e.g., acidic cannabinoid, neutral cannabinoid, acidic cannabinoid derivative, or neutral cannabinoid derivative) is performed by slowly cooling an aqueous phase comprising the cannabinoid or cannabinoid derivative to a temperature of about 0° C., about 1° C., about 2° C., about 3° C., about 4° C., about 5° C., about 6° C., about 7° C., about 8° C., about 9° C., about 10° C., about 11° C., about 12° C., about 13° C., about 14° C., about 15° C., about 16° C., about 17° C., about 18° C., about 19° C., about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., about 37° C., about 38° C., about 39° C., or about 40° C.

In some embodiments, crystallization of the cannabinoid or cannabinoid derivative (e.g., acidic cannabinoid, neutral cannabinoid, acidic cannabinoid derivative, or neutral cannabinoid derivative) occurs at a pH below about 12.0. In some embodiments, crystallization of the cannabinoid or cannabinoid derivative (e.g., acidic cannabinoid, neutral cannabinoid, acidic cannabinoid derivative, or neutral cannabinoid derivative) occurs a pH below about 2.0. In some embodiments, crystallization of the cannabinoid or cannabinoid derivative (e.g., acidic cannabinoid, neutral cannabinoid, acidic cannabinoid derivative, or neutral cannabinoid derivative) occurs at a pH of about 1.0 to about 12.0. In some embodiments, crystallization of the cannabinoid or cannabinoid derivative (e.g., acidic cannabinoid, neutral cannabinoid, acidic cannabinoid derivative, or neutral cannabinoid derivative) occurs at a pH of about 1.0, about 1.1, about 1.2, about 1.3, about 1.4, about 1.5, about 1.6, about 1.7, about 1.8, about 1.9, about 2.0, about 2.1, about 2.2, about 2.3, about 2.4, about 2.5, about 2.6, about 2.7, about 2.8, about 2.9, about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, about 4.5, about 4.6, about 4.7, about 4.8, about 4.9, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.7, about 6.8, about 6.9, about 7.0, about 7.1, about 7.2, about 7.3, about 7.4, about 7.5, about 7.6, about 7.7, about 7.8, about 7.9, about 8.0, about 8.1, about 8.2, about 8.3, about 8.4, about 8.5, about 8.6, about 8.7, about 8.8, about 8.9, about 9.0, about 9.1, about 9.2, about 9.3, about 9.4, about 9.5, about 9.6, about 9.7, about 9.8, about 9.9, or about 10.0, about 10.1, about 10.2, about 10.3, about 10.4, about 10.5, about 10.6, about 10.7, about 10.8, about 10.9, about 11.0, about 11.1, about 11.2, about 11.3, about 11.4, about 11.5, about 11.6, about 11.7, about 11.8, about 11.9, or about 12.0. In some embodiments, crystallization of the cannabinoid or cannabinoid derivative (e.g., acidic cannabinoid, neutral cannabinoid, acidic cannabinoid derivative, or neutral cannabinoid derivative) occurs at a pH of about 1.0 to about 10.0.

In some embodiments, the yield of the cannabinoid or cannabinoid derivative preparation recovered after crystallization of the cannabinoid or cannabinoid derivative (e.g., acidic cannabinoid, neutral cannabinoid, acidic cannabinoid derivative, or neutral cannabinoid derivative) is at least about 50%. In some embodiments, the yield of the cannabinoid or cannabinoid derivative preparation recovered after crystallization of the cannabinoid or cannabinoid derivative (e.g., acidic cannabinoid, neutral cannabinoid, acidic cannabinoid derivative, or neutral cannabinoid derivative) is at least about 70%. In some embodiments, the yield of the cannabinoid or cannabinoid derivative preparation recovered after crystallization of the cannabinoid or cannabinoid derivative (e.g., acidic cannabinoid, neutral cannabinoid, acidic cannabinoid derivative, or neutral cannabinoid derivative) is at least about 80%. In some embodiments, the yield of the cannabinoid or cannabinoid derivative preparation recovered after crystallization of the cannabinoid or cannabinoid derivative (e.g., acidic cannabinoid, neutral cannabinoid, acidic cannabinoid derivative, or neutral cannabinoid derivative) is at least about 90%. In some embodiments, the yield of the cannabinoid or cannabinoid derivative preparation recovered after crystallization of the cannabinoid or cannabinoid derivative (e.g., acidic cannabinoid, neutral cannabinoid, acidic cannabinoid derivative, or neutral cannabinoid derivative) is at least about 95%. In some embodiments, the yield of the cannabinoid or cannabinoid derivative preparation recovered after crystallization of the cannabinoid or cannabinoid derivative (e.g., acidic cannabinoid, neutral cannabinoid, acidic cannabinoid derivative, or neutral cannabinoid derivative) is at least about 96%. In some embodiments, the yield of the cannabinoid or cannabinoid derivative preparation recovered after crystallization of the cannabinoid or cannabinoid derivative (e.g., acidic cannabinoid, neutral cannabinoid, acidic cannabinoid derivative, or neutral cannabinoid derivative) is at least about 97%. In some embodiments, the yield of the cannabinoid or cannabinoid derivative preparation recovered after crystallization of the cannabinoid or cannabinoid derivative (e.g., acidic cannabinoid, neutral cannabinoid, acidic cannabinoid derivative, or neutral cannabinoid derivative) is at least about 98%. In some embodiments, the yield of the cannabinoid or cannabinoid derivative preparation recovered after crystallization of the cannabinoid or cannabinoid derivative (e.g., acidic cannabinoid, neutral cannabinoid, acidic cannabinoid derivative, or neutral cannabinoid derivative) is at least about 99%. In some embodiments, the yield of the cannabinoid or cannabinoid derivative preparation recovered after crystallization of the cannabinoid or cannabinoid derivative (e.g., acidic cannabinoid, neutral cannabinoid, acidic cannabinoid derivative, or neutral cannabinoid derivative) is between about 50% and about 100%. In some embodiments, the yield of the cannabinoid or cannabinoid derivative preparation recovered after crystallization of the cannabinoid or cannabinoid derivative (e.g., acidic cannabinoid, neutral cannabinoid, acidic cannabinoid derivative, or neutral cannabinoid derivative) is about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about, 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the cannabinoid or cannabinoid derivative preparation afforded by crystallization or precipitation is recovered by filtration. Any appropriate filtration method may be used. In some embodiments, the cannabinoid or cannabinoid derivative preparation is recovered by vacuum filtration. In some embodiments, recovered liquid is subjected to another round of crystallization or precipitation and then vacuum filtered again.

In some embodiments, the methods of the present disclosure provide a substantially pure cannabinoid or cannabinoid derivative preparation. In some embodiments, the purity of the cannabinoid or cannabinoid derivative preparation is determined by chromatographic purity (by area normalization). In some embodiments, the cannabinoid or cannabinoid derivative preparation has a chromatographic purity of about 50% to about 100%. In some embodiments, the cannabinoid or cannabinoid derivative preparation has a chromatographic purity of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about, 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% by area normalization.

In some embodiments, crystallization of the cannabinoid or cannabinoid derivative (e.g., acidic cannabinoid, neutral cannabinoid, acidic cannabinoid derivative, or neutral cannabinoid derivative) removes all or substantially all of the byproducts and/or impurities (e.g., olivetol, olivetolic acid, HTAL, PDAL, hexanoic acid) from the cannabinoid or cannabinoid derivative preparation. In some embodiments, crystallization of the cannabinoid or cannabinoid derivative (e.g., acidic cannabinoid, neutral cannabinoid, acidic cannabinoid derivative, or neutral cannabinoid derivative) removes at least 95% of the byproducts and/or impurities from the cannabinoid or cannabinoid derivative preparation. In some embodiments, crystallization of the cannabinoid or cannabinoid derivative (e.g., acidic cannabinoid, neutral cannabinoid, acidic cannabinoid derivative, or neutral cannabinoid derivative) removes about 50% to about 100% of the byproducts and/or impurities from the cannabinoid or cannabinoid derivative preparation. In some embodiments, crystallization of the cannabinoid or cannabinoid derivative (e.g., acidic cannabinoid, neutral cannabinoid, acidic cannabinoid derivative, or neutral cannabinoid derivative) removes about 50% to about 100% of the byproducts and/or impurities from the cannabinoid or cannabinoid derivative preparation. In some embodiments, crystallization of the cannabinoid or cannabinoid derivative (e.g., acidic cannabinoid, neutral cannabinoid, acidic cannabinoid derivative, or neutral cannabinoid derivative) removes about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% of the byproducts and/or impurities from the cannabinoid or cannabinoid derivative preparation.

In some embodiments, crystallization of the cannabinoid or cannabinoid derivative (e.g., acidic cannabinoid, neutral cannabinoid, acidic cannabinoid derivative, or neutral cannabinoid derivative) removes about substantially most (e.g., <5%) of the olivetol, olivetolic acid, PDAL, and HTAL, while retaining about 95% of the cannabinoid or cannabinoid derivativein the resulting preparation. In some embodiments, the cannabinoid or cannabinoid derivative in the preparation is a neutral cannabinoid or neutral cannabinoid derivative. In some embodiments, the neutral cannabinoid or neutral cannabinoid derivative in the preparation is tetrahydrocannabinol (THC), cannabidiol (CBD), or cannabigerol (CBG). In some embodiments, the cannabinoid or cannabinoid derivative in the preparation is an acidic cannabinoid or acidic cannabinoid derivative. In some embodiments, the acidic cannabinoid or acidic cannabinoid derivative in the preparation is cannabigerolic acid (CBGA), tetrahydrocannabinolic acid (THCA), or cannabidiolic acid (CBDA).

In some aspects, the cannabinoid or cannabinoid derivative preparation is recovered by crystallizing or precipitating the acidic cannabinoid or acidic cannabinoid derivative as disclosed herein and then decarboxylating the acidic cannabinoid or acidic cannabinoid derivative. In some embodiments, the acidic cannabinoid or acidic cannabinoid derivative is decarboxylated in a dry form. In some embodiments, the acidic cannabinoid or acidic cannabinoid derivative is decarboxylated in an aqueous solution.

### Cannabinoid or Cannabinoid Derivative Preparations

The disclosure provides for preparations preparable by any of the methods disclosed herein. The disclosure also provides for cannabinoid or cannabinoid derivative preparations prepared by any of the methods disclosed herein. In some embodiments, the disclosure provides for a cannabinoid preparation. In some embodiments, the disclosure provides for a cannabinoid derivative preparation. In some embodiments, the disclosure provides for an acidic cannabinoid preparation. In some embodiments, the disclosure provides for an acidic cannabinoid derivative preparation. In some embodiments, the disclosure provides for a neutral cannabinoid preparation. In some embodiments, the disclosure provides for a neutral cannabinoid derivative preparation.

The purity of the cannabinoid or cannabinoid derivative preparation recovered by the methods of the instant disclosure may be determined using any appropriate means in the art. In some embodiments, the purity of the cannabinoid or cannabinoid derivative preparation is measured by the percentage of the dry weight. In some embodiments, the dry weight of the cannabinoid or cannabinoid derivative comprises over 99% of the total weight of the preparation.

In some embodiments, the purity of the recovered cannabinoid or cannabinoid derivative preparation is measured by elemental (combustion) analysis. This method requires that solids are first dried in a vacuum oven to a constant weight to remove any residual, volatile solvents.

In some embodiments, the purity of the recovered cannabinoid or cannabinoid derivative preparation is measured by ICP/MS (Inductively Coupled Mass Spectrometry).

In some embodiments, the purity of the cannabinoid or cannabinoid derivative preparation produced and recovered using the methods of the instant disclosure is between about 70% and about 100% purity. In some embodiments, the cannabinoid or cannabinoid derivative preparation is about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, about 99.1%, about 99.2%, about 99.3%, about 99.4%, about 99.5%, about 99.6%, about 99.7%, about 99.8%, about 99.9%, about 99.91%, about 99.92%, about 99.93%, about 99.94%, about 99.95%, about 99.96%, about 99.97%, about 99.98%, about 99.99%, or about 100% pure.

In some embodiments, the cannabinoid or cannabinoid derivative preparation comprises at least 85 weight % of a cannabinoid or cannabinoid derivative. In some embodiments, the cannabinoid or cannabinoid derivative preparation comprises at least 90 weight % of a cannabinoid or cannabinoid derivative. In some embodiments, the cannabinoid or cannabinoid derivative preparation comprises at least 95 weight % of a cannabinoid or cannabinoid derivative. In some embodiments, the cannabinoid or cannabinoid derivative preparation comprises at least 70 weight %, 71 weight %, 72 weight %, 73 weight %, 74 weight %, 75 weight %, 76 weight %, 77 weight %, 78 weight %, 79 weight %, 80 weight %, 81 weight %, 82 weight %, 83 weight %, 84 weight %, 85 weight %, 86 weight %, 87 weight %, 88 weight %, 89 weight %, 90 weight %, 91 weight %, 92 weight %, 93 weight %, 94 weight %, 95 weight %, 96 weight %, 97 weight %, 98 weight %, or 99 weight % of the cannabinoid or cannabinoid derivative.

In some embodiments, the cannabinoid or cannabinoid derivative preparation produced and recovered using the method of the instant disclosure is free of impurities and byproducts. In some embodiments, the cannabinoid or cannabinoid derivative preparation produced and recovered using the method of the instant disclosure is substantially free of impurities and byproducts. In some embodiments, the cannabinoid or cannabinoid derivative preparation produced and recovered using the method of the instant disclosure contains less than about 5% of impurities and byproducts. In some embodiments, the cannabinoid or cannabinoid derivative preparation produced and recovered using the methods of the instant disclosure contains about 0.1%, about 0.2%, about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, about 1.7%, about 1.8%, about 1.9%, about 2.0%, about 2.1%, about 2.2%, about 2.3%, about 2.4%, about 2.5%, about 2.6%, about 2.7%, about 2.8%, about 2.9%, about 3.0%, about 3.1%, about 3.2%, about 3.3%, about 3.4%, about 3.5%, about 3.6%, about 3.7%, about 3.8%, about 3.9%, about 4.0%, about 4.1%, about 4.2%, about 4.3%, about 4.4%, about 4.5%, about 4.6%, about 4.7%, about 4.8%, about 4.9%, or about 5.0% impurities and byproducts. In some embodiments, the byproducts and/or impurities may include, but are not limited to, hexanoyl triacetic acid lactone (HTAL), pentyl diacetic acid lactone (PDAL), olivetol, olivetolic acid, and hexanoic acid.

In some embodiments, the cannabinoid or cannabinoid derivative preparation is white or substantially white. In some embodiments, the cannabinoid or cannabinoid derivative preparation is a pale color. In some embodiments, the cannabinoid or cannabinoid derivative preparation is a pale yellow. In some embodiments, the cannabinoid or cannabinoid derivative preparation is a crystalline solid.

In some embodiments, the cannabinoid or cannabinoid derivative preparation is a crystalline solid at about 25° C. In some embodiments, the cannabinoid or cannabinoid derivative preparation is a crystalline solid at about 25° C. to about 40° C. In some embodiments, the cannabinoid or cannabinoid derivative preparation is a crystalline solid at about 25° C., about 26° C., about 27° C., about 28° C., about 29° C., about 30° C. about 31° C., about 32° C., about 33° C., about 34° C., about 35° C., about 36° C., about 37° C., about 38° C., about 39° C., or about 40° C. In some embodiments, the cannabinoid or cannabinoid derivative preparation is a white or substantially white crystalline solid at about 25° C. In some embodiments, the cannabinoid or cannabinoid preparation is a white or substantially white crystalline solid at about 25° C. to about 40° C. In some embodiments, the cannabinoid or cannabinoid derivative preparation is a white or substantially white crystalline solid at about 25° C., about 26° C., about 27° C., about 28° C., about 29° C., about 30° C. about 31° C., about 32° C., about 33° C., about 34° C., about 35° C., about 36° C., about 37° C., about 38° C., about 39° C., or about 40° C. In some embodiments, the cannabinoid or cannabinoid derivative preparation is a pale yellow crystalline solid at about 25° C. In some embodiments, the cannabinoid or cannabinoid derivative preparation is a pale yellow solid at about 25° C. to about 40° C. In some embodiments, the cannabinoid or cannabinoid derivative preparation is pale yellow crystalline solid at about 25° C., about 26° C., about 27° C., about 28° C., about 29° C., about 30° C. about 31° C., about 32° C., about 33° C., about 34° C., about 35° C., about 36° C., about 37° C., about 38° C., about 39° C., or about 40° C.

In some embodiments, the cannabinoid or cannabinoid derivative preparation is a crystalline solid that melts around 70° C. In some embodiments, the cannabinoid or cannabinoid derivative preparation is a crystalline solid that melts between about 60° C. to about 80° C. In some embodiments, the cannabinoid or cannabinoid derivative preparation is a crystalline solid that melts around 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C. In some embodiments, the cannabinoid or cannabinoid derivative preparation is a white or substantially white crystalline solid that melts around 70° C. In some embodiments, the cannabinoid or cannabinoid derivative preparation is a white or substantially white crystalline solid that melts between about 60° C. to about 80° C. In some embodiments, the cannabinoid or cannabinoid derivative preparation is a white or substantially white crystalline solid that melts around 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C. In some embodiments, the cannabinoid or cannabinoid derivative preparation is a pale yellow crystalline solid that melts around 70° C. In some embodiments, the cannabinoid or cannabinoid derivative preparation is a pale yellow crystalline solid that melts between about 60° C. to about 80° C. In some embodiments, the cannabinoid or cannabinoid derivative preparation is a pale yellow crystalline solid that melts around 60° C., about 61° C., about 62° C., about 63° C., about 64° C., about 65° C., about 66° C., about 67° C., about 68° C., about 69° C., about 70° C., about 71° C., about 72° C., about 73° C., about 74° C., about 75° C., about 76° C., about 77° C., about 78° C., about 79° C., or about 80° C.

In some embodiments, the cannabinoid or cannabinoid derivative preparation recovered after crystallization of the cannabinoid or cannabinoid derivative is a white or substantially white crystalline solid at temperatures between about 25° C. and about 40° C. that has a purity of between about 50% and about 100%. In some embodiments, the cannabinoid or cannabinoid derivative preparation recovered after crystallization of the cannabinoid or cannabinoid derivative is a white or substantially white crystalline solid at a temperature of about 25° C., about 26° C., about 27° C., about 28° C., about 29° C., about 30° C. about 31° C., about 32° C., about 33° C., about 34° C., about 35° C., about 36° C., about 37° C., about 38° C., about 39° C., or about 40° C. and has a purity of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about, 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the cannabinoid or cannabinoid derivative preparation recovered after crystallization of the cannabinoid or cannabinoid derivative is a pale yellow crystalline solid at temperatures between about 25° C. and about 40° C. that has a purity of between about 50% and about 100%. In some embodiments, the cannabinoid or cannabinoid derivative preparation recovered after crystallization of the cannabinoid or cannabinoid derivative is a pale yellow crystalline solid at a temperature of about 25° C., about 26° C., about 27° C., about 28° C., about 29° C., about 30° C. about 31° C., about 32° C., about 33° C., about 34° C., about 35° C., about 36° C., about 37° C., about 38° C., about 39° C., or about 40° C. and has a purity of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about, 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100%.

In some embodiments, the cannabinoid or cannabinoid derivative preparation recovered after crystallization of the cannabinoid or cannabinoid derivative is a pale yellow crystalline solid, having a chromatographic purity of about 98% by area normalization with a melting point of about 70° C. In some embodiments, the cannabinoid or cannabinoid derivative is tetrahydrocannabinol (THC), cannabidiol (CBD), or cannabigerol (CBG). In some embodiments, the cannabinoid or cannabinoid derivative preparation possesses CBD<0.5% w/w THC=1.0%s w/w CBN<1.0% w/w.

In some embodiments, the cannabinoid or cannabinoid derivative in the preparation is a neutral cannabinoid or neutral cannabinoid derivative. In some embodiments, the neutral cannabinoid or neutral cannabinoid derivative in the preparation is tetrahydrocannabinol (THC), cannabidiol (CBD), or cannabigerol (CBG). In some embodiments, the cannabinoid or cannabinoid derivative in the preparation is an acidic cannabinoid or acidic cannabinoid derivative. In some embodiments, the acidic cannabinoid or acidic cannabinoid derivative in the preparation is cannabigerolic acid (CBGA), tetrahydrocannabinolic acid (THCA), or cannabidiolic acid (CBDA).

### Cannabinoids and Cannabinoid Derivatives of the Methods or Cannabinoid or Cannabinoid Derivative Preparations of the Disclosure

The present disclosure provides methods and preparations comprising a cannabinoid or a cannabinoid derivative. The cannabinoid may be a neutral or an acidic cannabinoid and the cannabinoid derivative may be a neutral or an acidic cannabinoid derivative. These cannabinoids and cannabinoid derivatives of the methods and preparations of the disclosure may be produced using the methods and modified host cells detailed in WO2018/200888 filed Apr. 27, 2018 and WO 2020/069214 filed Sep. 26, 2019, and PCT/US2020/051261 filed Sep. 17, 2020. Acidic cannabinoids and acidic cannabinoid derivatives may be decarboxylated via the methods disclosed herein to afford neutral cannabinoids and neutral cannabinoid derivatives.

Cannabinoids or cannabinoid derivatives of the methods or cannabinoid or cannabinoid derivative preparations of the present disclosure may include, but are not limited to, cannabichromene (CBC) type (e.g., cannabichromenic acid), cannabigerol (CBG) type (e.g., cannabigerolic acid), cannabidiol (CBD) type (e.g., cannabidiolic acid), Δ⁹-trans-tetrahydrocannabinol (Δ⁹-THC) type (e.g., Δ⁹-tetrahydrocannabinolic acid), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol (CBT) type, derivatives of any of the foregoing, and others as listed in Elsohly M. A. and Slade D., Life Sci. 2005 Dec. 22; 78(5):539-48. Epub 2005 Sep. 30. In some embodiments, the cannabinoid or cannabinoid derivative is a neutral cannabinoid or neutral cannabinoid derivative. In some embodiments, the cannabinoid or cannabinoid derivative is an acidic cannabinoid or acidic cannabinoid derivative.

Neutral cannabinoids or neutral cannabinoid derivatives of the methods or cannabinoid or cannabinoid derivative preparations of the present disclosure may include, but are not limited to, cannabichromene (CBC) type (e.g., cannabichromene), cannabigerol (CBG) type (e.g., cannabigerol), cannabidiol (CBD) type (e.g., cannabidiol), Δ⁹-trans-tetrahydrocannabinol (Δ⁹-THC) type (e.g., Δ⁹-tetrahydrocannabinol), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol (CBT) type, derivatives of any of the foregoing, and others as listed in Elsohly M. A. and Slade D., Life Sci. 2005 Dec. 22;78(5):539-48. Epub 2005 Sep. 30.

Acidic cannabinoids or acidic cannabinoid derivatives of the methods or cannabinoid or cannabinoid derivative preparations of the present disclosure may include, but are not limited to, cannabichromene (CBC) type (e.g., cannabichromenic acid), cannabigerol (CBG) type (e.g., cannabigerolic acid), cannabidiol (CBD) type (e.g., cannabidiolic acid), Δ⁹-trans-tetrahydrocannabinol (Δ⁹-THC) type (e.g., Δ⁹-tetrahydrocannabinolic acid), Δ⁸-trans-tetrahydrocannabinol (Δ⁸-THC) type, cannabicyclol (CBL) type, cannabielsoin (CBE) type, cannabinol (CBN) type, cannabinodiol (CBND) type, cannabitriol (CBT) type, derivatives of any of the foregoing, and others as listed in Elsohly M. A. and Slade D., Life Sci. 2005 Dec. 22;78(5):539-48. Epub 2005 Sep. 30.

Neutral cannabinoids or neutral cannabinoid derivatives of the methods or cannabinoid or cannabinoid derivative preparations of the present disclosure may also include, but are not limited to, cannabigerolic acid monomethylether (CBGAM), cannabigerol (CBG), cannabigerol monomethylether (CBGM), cannabigerovarin (CBGV), cannabichromene (CBC), cannabichromevarin (CBCV), cannabidiol (CBD), cannabidiol monomethylether (CBDM), cannabidiol-C₄ (CBD-C₄), cannabidivarin (CBDV), cannabidiorcol (CBD-C₁), Δ⁹-tetrahydrocannabinol (THC), Δ⁹-tetrahydrocannabinol-C₄ (THC-C₄), Δ⁹-tetrahydrocannabivarin (THCV), Δ⁹-tetrahydrocannabiorcol (THC-C₁), Δ⁷-cis-iso-tetrahydrocannabivarin, Δ⁸-tetrahydrocannabinol (Δ⁸-THC), cannabicyclol (CBL), cannabicyclovarin (CBLV), cannabielsoin (CBE), cannabinol (CBN), cannabinol methylether (CBNM), cannabinol-C₄, (CBN-C₄), cannabivarin (CBV), cannabinol-C₂ (CNB-C₂), cannabiorcol (CBN-C₁), cannabinodiol (CBND), cannabinodivarin (CBVD), cannabitriol (CBT), 10-ethyoxy-9-hydroxy-delta-6a-tetrahydrocannabinol, 8,9-dihydroxyl-delta-6a-tetrahydrocannabinol, cannabitriolvarin (CBTV), dehydrocannabifuran (DCBF), cannabifuran (CBF), cannabichromanon (CBCN), cannabicitran (CBT), 10-oxo-delta-6a-tetrahydrocannabinol (OTHC), delta-9-cis-tetrahydrocannabinol (cis-THC), 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsol (CBR), trihydroxy-delta-9-tetrahydrocannabinol (triOH-THC), CBG-hydrocinnamic acid (2-[(2E)-3,7-dimethylocta-2,6-dien-1-yl]-5-(2-phenylethyl)benzene-1,3-diol), CBD-hydrocinnamic acid (2-[3-methyl-6-(prop-1-en-2-yl)cyclohex-2-en-1-yl]-5-(2-phenylethyl)benzene-1,3-diol), THC-hydrocinnamic acid (6,6,9-trimethyl-3-(2-phenylethyl)-6H,6aH,7H,8H,10aH-benzo[c]isochromen-1-ol, perrottetinene), and derivatives of any of the foregoing.

Acidic cannabinoids or acidic cannabinoid derivatives of the methods or cannabinoid or cannabinoid derivative preparations of the present disclosure may also include, but are not limited to, cannabigerolic acid (CBGA), cannabigerovarinic acid (CBGVA), cannabichromenic acid (CBCA), cannabichromevarinic acid (CBCVA), cannabidiolic acid (CBDA), cannabidiolic acid-C₄ (CBDA-C₄), cannabidivarinic acid (CBDVA), cannabidiorcolic acid (CBDA-C₁), Δ⁹-tetrahydrocannabinolic acid A (THCA-A), Δ⁹-tetrahydrocannabinolic acid B (THCA-B), Δ⁹-tetrahydrocannabinolic acid-C₄ (THCA-C₄), Δ⁹-tetrahydrocannabivarinic acid (THCVA), Δ⁹-tetrahydrocannabiorcolic acid (THCA-C₁), Δ⁸-tetrahydrocannabinolic acid (Δ⁸-THCA), cannabicyclovarinic acid (CBLVA), cannabicyclolic acid (CBLA), cannabielsoic acid A (CBEA-A), cannabielsoic acid B (CBEA-B), cannabielsoinic acid, cannabicitranic acid, cannabinolic acid (CBNA), cannabinolic acid-C₄, (CBNA-C₄), cannabivarinic acid (CBVA), cannabinolic acid-C₂ (CNBA-C₂), cannabiorcolic acid (CBNA-C₁), cannabinodiolic acid (CBNDA), cannabinodivarinic acid (CBVDA), cannabitriolic acid (CBTA), 10-ethyoxy-9-hydroxy-delta-6a-tetrahydrocannabinolic acid, 8,9-dihydroxyl-delta-6a-tetrahydrocannabinolic acid, cannabitriolvarinic acid (CBTVA), acidic form of dehydrocannabifuran, acidic form of cannabifuran, acidic form of cannabichromanon, acidic form of cannabicitran, 10-oxo-delta-6a-tetrahydrocannabinolic acid (OTHCA), delta-9-cis-tetrahydrocannabinolic acid (cis-THCA), acidic form of 3,4,5,6-tetrahydro-7-hydroxy-alpha-alpha-2-trimethyl-9-n-propyl-2,6-methano-2H-1-benzoxocin-5-methanol (OH-iso-HHCV), cannabiripsolic acid (CBRA), trihydroxy-delta-9-tetrahydrocannabinolic acid (triOH-THCA), CBGA-hydrocinnamic acid (3-[(2E)-3,7-dimethylocta-2,6-dien-1-yl]-2,4-dihydroxy-6-(2-phenylethyl)benzoic acid), CBDA-hydrocinnamic acid (2,4-dihydroxy-3-[3-methyl-6-(prop-1-en-2-yl)cyclohex-2-en-1-yl]-6-(2-phenylethyl)benzoic acid), THCA-hydrocinnamic acid (1-hydroxy-6,6,9-trimethyl-3-(2-phenylethyl)-6H,6aH,7H,8H,10aH-benzo[c]isochromene-2-carboxylic acid), and derivatives of any of the foregoing.

In some embodiments, the neutral cannabinoid of the methods or cannabinoid preparations of the present disclosure is cannabigerol, Δ⁹-tetrahydrocannabinol, Δ⁸-tetrahydrocannabinol, cannabidiol, cannabichromene, cannabinol, cannabidivarin, tetrahydrocannabivarin, cannabichromevarin, cannabigerovarin, cannabicyclol, cannabielsoin, or cannabicitran.

In some embodiments, the acidic cannabinoid of the methods or cannabinoid preparations of the present disclosure is cannabigerolic acid, Δ⁹-tetrahydrocannabinolic acid, Δ⁸-tetrahydrocannabinolic acid, cannabidiolic acid, cannabichromenic acid, cannabinolic acid, cannabidivarinic acid, tetrahydrocannabivarinic acid, cannabichromevarinic acid, cannabigerovarinic acid, cannabicyclolic acid, cannabielsoinic acid, or cannabicitranic acid.

In some embodiments, the neutral cannabinoid of the methods or cannabinoid preparations of the present disclosure is tetrahydrocannabinol (THC), cannabidiol (CBD), or cannabigerol (CBG).

In some embodiments, the acidic cannabinoid of the methods or cannabinoid preparations of the present disclosure is cannabigerolic acid (CBGA), tetrahydrocannabinolic acid (THCA), or cannabidiolic acid (CBDA).

Additional neutral cannabinoids and neutral cannabinoid derivatives of the methods or cannabinoid or cannabinoid derivative preparations of the present disclosure may also include, but are not limited to, (6aR,10aR)-3-butyl-6,6,9-trimethyl-6a,7,8,10a-tetrahydro-6H-benzo[c]chromen-1-ol, (6aR,10aR)-6,6,9-trimethyl-3-(3-methylpentyl)-6a,7,8, 10a-tetrahydro-6H-benzo[c]chromen-1-ol, (6aR, 10aR)-6,6,9-trimethyl-3-(pent-4-en-1-yl)-6a,7, 8,10a-tetrahydro-6H-benzo[c]chromen-1-ol, (6aR, 10aR)-3-hexyl-6,6,9-trimethyl-6a,7, 8,10a-tetrahydro-6H-benzo[c]chromen-1-ol, (6aR,10aR)-3-(hex-5-yn-1-yl)-6,6,9-trimethyl-6a,7,8,10a-tetrahydro-6H-benzo[c]chromen-1-ol, and others as listed in Bow, E. W. and Rimoldi, J. M., “The Structure-Function Relationships of Classical Cannabinoids: CB1/CB2 Modulation,” Perspectives in Medicinal Chemistry 2016: 8 17-39 doi: 10.4137/PMC.S32171, incorporated by reference herein.

Additional acidic cannabinoids and acidic cannabinoid derivatives of the methods or cannabinoid or cannabinoid derivative preparations of the present disclosure may also include, but are not limited to, (6aR,10aR)-1-hydroxy-6,6,9-trimethyl-3-butyl-6a,7,8,10a-tetrahydro-6H-dibenzo[b,d]pyran-2-carboxylic acid, (6aR,10aR)-1-hydroxy-6,6, 9-trimethyl-3-(3-methylpentyl)-6a, 7,8,10a-tetrahydro-6H-dibenzo[b,d]pyran-2-carboxylic acid, (6aR,10aR)-1-hydroxy-6,6,9-trimethyl-3-(4-pentenyl)-6a,7,8,10a-tetrahydro-6H-dibenzo[b,d]pyran-2-carboxylic acid, (6aR,10aR)-1-hydroxy-6,6,9-trimethyl-3-hexyl-6a,7,8,10a-tetrahydro-6H-dibenzo[b,d]pyran-2-carboxylic acid, (6aR, 10aR)-1-hydroxy-6,6,9-trimethyl-3-(5-hexynyl)-6a,7,8,10a-tetrahydro-6H-dibenzo[b,d]pyran-2-carboxylic acid, and others as listed in Bow, E. W. and Rimoldi, J. M., “The Structure-Function Relationships of Classical Cannabinoids: CB1/CB2 Modulation,” Perspectives in Medicinal Chemistry 2016: 8 17-39 doi: 10.4137/PMC.S32171, incorporated by reference herein.

Additional acidic cannabinoids and acidic cannabinoid derivatives of the methods or cannabinoid or cannabinoid derivative preparations of the present disclosure may also include, but are not limited to, (E)-6-(but-3-yn-1-yl)-3-(3,7-dimethylocta-2,6-dien-1-yl)-2,4-dihydroxybenzoic acid, 6-((E)-but-1-en-1-yl)-3-((E)-3,7-dimethylocta-2,6-dien-1-yl)-2,4-dihydroxybenzoic acid, (E)-3-(3,7-dimethylocta-2,6-dien-1-yl)-2,4-dihydroxy-6-(pent-4-yn-1-yl)benzoic acid, (E)-3-(3,7-dimethylocta-2,6-dien-1-yl)-6-(hex-5-yn-1-yl)-2,4-dihydroxybenzoic acid, 3-((E)-3,7-dimethylocta-2,6-dien-1-yl)-6-((E)-hept-1-en-1-yl)-2,4-dihydroxybenzoic acid, 3-((E)-3,7-dimethylocta-2,6-dien-1-yl)-2,4-dihydroxy-6-((E)-oct-1-en-1-yl)benzoic acid, (E)-3-(3,7-dimethylocta-2,6-dien-1-yl)-2,4-dihydroxy-6-(3-phenylpropyl)benzoic acid, (E)-3-(3,7-dimethylocta-2,6-dien-1-yl)-2,4-dihydroxy-6-(4-phenylbutyl)benzoic acid, (E)-3-(3,7-dimethylocta-2,6-dien-1-yl)-2,4-dihydroxy-6-(5-phenylpentyl)benzoic acid, (E)-3-(3,7-dimethylocta-2,6-dien-1-yl)-2,4-dihydroxy-6-(6-phenylhexyl)benzoic acid, 3-[(2E)-3,7-dimethylocta-2,6-dien-1-yl]-6-(hexan-2-yl)-2,4-dihydroxybenzoic acid, 3-[(2E)-3,7-dimethylocta-2,6-dien-1-yl]-2,4-dihydroxy-6-(2-methylpentyl)benzoic acid, 3-[(2E)-3,7-dimethylocta-2,6-dien-1-yl]-2,4-dihydroxy-6-(3-methylpentyl)benzoic acid, 3-[(2E)-3,7-dimethylocta-2,6-dien-1-yl]-2,4-dihydroxy-6-(4-methylpentyl)benzoic acid, 3-[(2E)-3,7-dimethylocta-2,6-dien-1-yl]-2,4-dihydroxy-6-[(1E)-pent-1-en-1-yl]benzoic acid, 3-[(2E)-3,7-dimethylocta-2,6-dien-1-yl]-2,4-dihydroxy-6-[(2E)-pent-2-en-1-yl]benzoic acid, 3-[(2E)-3,7-dimethylocta-2,6-dien-1-yl]-2,4-dihydroxy-6-[(2E)-pent-3-en-1-yl]benzoic acid, 3-[(2E)-3,7-dimethylocta-2,6-dien-1-yl]-2,4-dihydroxy-6-(pent-4-en-1-yl)benzoic acid, 3-[(2E)-3,7-dimethylocta-2,6-dien-1-yl]-2,4-dihydroxy-6-propylbenzoic acid, 3-[(2E)-3,7-dimethylocta-2,6-dien-1-yl]-2,4-dihydroxy-6-butylbenzoic acid, 3-[(2E)-3,7-dimethylocta-2,6-dien-1-yl]-2,4-dihydroxy-6-hexylbenzoicacid, 3-[(2E)-3,7-dimethylocta-2,6-dien-1-yl]-2,4-dihydroxy-6-heptylbenzoic acid, 3-[(2E)-3,7-dimethylocta-2,6-dien-1-yl]-2,4-dihydroxy-6-octylbenzoic acid, 3-[(2E)-3,7-dimethylocta-2,6-dien-1-yl]-2,4-dihydroxy-6-nonanylbenzoic acid, 3-[(2E)-3,7-dimethylocta-2,6-dien-1-yl]-2,4-dihydroxy-6-decanylbenzoic acid, 3-[(2E)-3,7-dimethylocta-2,6-dien-1-yl]-2,4-dihydroxy-6-undecanylbenzoic acid, (E)-3-(3,7-dimethylocta-2,6-dien-1-yl)-2,4-dihydroxy-6-tridecylbenzoic acid, 6-(4-chlorobutyl)-3-[(2E)-3,7-dimethylocta-2,6-dien-1-yl]-2,4-dihydroxybenzoic acid, 3-[(2E)-3,7-dimethylocta-2,6-dien-1-yl]-2,4-dihydroxy-6-[4-(methylsulfanyl)butyl]benzoic acid, (E)-3-(3,7-dimethylocta-2,6-dien-1-yl)-2,4-dihydroxy-6-isopropylbenzoic acid, 6-((E)-2-carboxyvinyl)-3-((E)-3,7-dimethylocta-2,6-dien-1-yl)-2,4-dihydroxybenzoic acid, 6-((Z)-2-carboxyvinyl)-3-((E)-3,7-dimethylocta-2,6-dien-1-yl)-2,4-dihydroxybenzoic acid, (E)-6-(6-carboxyhexyl)-3-(3,7-dimethylocta-2,6-dien-1-yl)-2,4-dihydroxybenzoic acid, (E)-6-(7-carboxyheptyl)-3-(3,7-dimethylocta-2,6-dien-1-yl)-2,4-dihydroxybenzoic acid, (E)-6-(8-carboxyoctyl)-3-(3,7-dimethylocta-2,6-dien-1-yl)-2,4-dihydroxybenzoic acid, (E)-6-(10-carboxydecyl)-3-(3 ,7-dimethylocta-2,6-dien-1-yl)-2,4-dihydroxybenzoic acid, (E)-4-(3,7-dimethylocta-2,6-dien-1-yl)-3,5-dihydroxy-[1,1′-biphenyl]-2,2′-dicarboxylic acid, (E)-4-(3,7-dimethylocta-2,6-dien-1-yl)-3,5-dihydroxy-[1,1′-biphenyl]-2,3′-dicarboxylic acid, (E)-4-(3,7-dimethylocta-2,6-dien-1-yl)-3,5-dihydroxy-[1,1′-biphenyl]-2,4′-dicarboxylic acid, (E)-4-(3,7-dimethylocta-2,6-dien-1-yl)-3,5-dihydroxy-[1,1′-biphenyl]-2,3′,5′-tricarboxylic acid, (E)-3-(3,7-dimethylocta-2,6-dien-1-yl)-2,4-dihydroxy-6-(4-hydroxybutyl)benzoic acid, (E)-6-(4-aminobutyl)-3-(3,7-dimethylocta-2,6-dien-1-yl)-2,4-dihydroxybenzoic acid, (E)-6-(4-cyanobutyl)-3-(3,7-dimethylocta-2,6-dien-1-yl)-2,4-dihydroxybenzoic acid, (E)-3-(3,7-dimethylocta-2,6-dien-1-yl)-2,4-dihydroxy-6-(3-methylhexan-2-yl)benzoic acid, (E)-6-(2-carboxyethyl)-3-(3,7-dimethylocta-2,6-dien-1-yl)-2,4-dihydroxybenzoic acid, (E)-6-(carboxymethyl)-3-(3,7-dimethylocta-2,6-dien-1-yl)-2,4-dihydroxybenzoic acid, (E)-6-(3-carboxypropyl)-3-(3,7-dimethylocta-2,6-dien-1-yl)-2,4-dihydroxybenzoic acid, (E)-4-(3,7-dimethylocta-2,6-dien-1-yl)-3,5-dihydroxyphthalic acid, (E)-6-(4-carboxybutyl)-3-(3,7-dimethylocta-2,6-dien-1-yl)-2,4-dihydroxybenzoic acid, (E)-6-(5-carboxypentyl)-3-(3,7-dimethylocta-2,6-dien-1-yl)-2,4-dihydroxybenzoic acid, (E)-3-(3,7-dimethylocta-2,6-dien-1-yl)-2,4-dihydroxy-6-pentylbenzoic acid, and 3-[(2E)-3,7-dimethylocta-2,6-dien-1-yl]-2,4-dihydroxy-6-methylbenzoic acid.

Additional neutral cannabinoids and neutral cannabinoid derivatives of the methods or preparations of the present disclosure may also include, but are not limited to, (E)-5-(but-3-yn-1-yl)-2-(3,7-dimethylocta-2,6-dien-1-yl)benzene-1,3-diol, 5-((E)-but-1-en-1-yl)-2-((E)-3,7-dimethylocta-2,6-dien-1-yl)benzene-1,3-diol, (E)-2-(3,7-dimethylocta-2,6-dien-1-yl)-5-(pent-4-yn-1-yl)benzene-1,3-diol, (E)-2-(3,7-dimethylocta-2,6-dien-1-yl)-5-(hex-5-yn-1-yl)benzene-1,3-diol, 2-((E)-3,7-dimethylocta-2,6-dien-1-yl)-5-((E)-hept-1-en-1-yl)benzene-1,3-diol, 2-((E)-3,7-dimethylocta-2,6-dien-1-yl)-5-((E)-oct-1-en-1-yl)benzene-1,3-diol, (E)-2-(3,7-dimethylocta-2,6-dien-1-yl)-5-(3-phenylpropyl)benzene-1,3-diol, (E)-2-(3,7-dimethylocta-2,6-dien-1-yl)-5-(4-phenylbutyl)benzene-1,3-diol, (E)-2-(3,7-dimethylocta-2,6-dien-1-yl)-5-(5-phenylpentyl)benzene-1,3-diol, (E)-2-(3,7-dimethylocta-2,6-dien-1-yl)-5-(6-phenylhexyl)benzene-1,3-diol, (E)-2-(3,7-dimethylocta-2,6-dien-1-yl)-5-(hexan-2-yl)benzene-1,3-diol, (E)-2-(3,7-dimethylocta-2,6-dien-1-yl)-5-(2-methylpentyl)benzene-1,3-diol, (E)-2-(3,7-dimethylocta-2,6-dien-1-yl)-5-(3-methylpentyl)benzene-1,3-diol, (E)-2-(3,7-dimethylocta-2,6-dien-1-yl)-5-(4-methylpentyl)benzene-1,3-diol, 2-((E)-3,7-dimethylocta-2,6-dien-1-yl)-5-((E)-pent-1-en-1-yl)benzene-1,3-diol, 2-((E)-3,7-dimethylocta-2,6-dien-1-yl)-5-((E)-pent-2-en-1-yl)benzene-1,3-diol, 2-((E)-3,7-dimethylocta-2,6-dien-1-yl)-5-((E)-pent-3-en-1-yl)benzene-1,3-diol, 2-((E)-3,7-dimethylocta-2,6-dien-1-yl)-5-((E)-pent-4-en-1-yl)benzene-1,3-diol, (E)-2-(3,7-dimethylocta-2,6-dien-1-yl)-5-propylbenzene-1,3-diol, (E)-5-butyl-2-(3,7-dimethylocta-2,6-dien-1-yl)benzene-1,3-diol, (E)-2-(3,7-dimethylocta-2,6-dien-1-yl)-5-hexylbenzene-1,3-diol, (E)-2-(3,7-dimethylocta-2,6-dien-1-yl)-5-heptylbenzene-1,3-diol, (E)-2-(3,7-dimethylocta-2,6-dien-1-yl)-5-octylbenzene-1,3-diol, (E)-2-(3,7-dimethylocta-2,6-dien-1-yl)-5-nonylbenzene-1,3-diol, (E)-5-decyl-2-(3,7-dimethylocta-2,6-dien-1-yl)benzene-1,3-diol, (E)-2-(3,7-dimethylocta-2,6-dien-1-yl)-5-undecylbenzene-1,3-diol, (E)-2-(3,7-dimethylocta-2,6-dien-1-yl)-5-tridecylbenzene-1,3-diol, (E)-5-(4-chlorobutyl)-2-(3,7-dimethylocta-2,6-dien-1-yl)benzene-1,3-diol, (E)-2-(3,7-dimethylocta-2,6-dien-1-yl)-5-(4-(methylthio)butyl)benzene-1,3-diol, (E)-2-(3,7-dimethylocta-2,6-dien-1-yl)-5-isopropylbenzene-1,3-diol, (E)-3-(4-((E)-3,7-dimethylocta-2,6-dien-1-yl)-3,5-dihydroxyphenyl)acrylic acid, (Z)-3-(4-((E)-3,7-dimethylocta-2,6-dien-1-yl)-3,5-dihydroxyphenyl)acrylic acid, (E)-7-(4-(3,7-dimethylocta-2,6-dien-1-yl)-3,5-dihydroxyphenyl)heptanoic acid, (E)-7-(4-(3,7-dimethylocta-2,6-dien-1-yl)-3,5-dihydroxyphenyl)octanoic acid, (E)-7-(4-(3,7-dimethylocta-2,6-dien-1-yl)-3,5-dihydroxyphenyl)nonanoic acid, (E)-7-(4-(3,7-dimethylocta-2,6-dien-1-yl)-3,5-dihydroxyphenyl)undecanoic acid, (E)-4′-(3,7-dimethylocta-2,6-dien-1-yl)-3′,5′-dihydroxy-[1,1′-biphenyl]-2-carboxylic acid, (E)-4′-(3,7-dimethylocta-2,6-dien-1-yl)-3′,5′-dihydroxy-[1,1′-biphenyl]-3-carboxylic acid, (E)-4′-(3,7-dimethylocta-2,6-dien-1-yl)-3′,5′-dihydroxy-[1,1′-biphenyl]-4-carboxylic acid, (E)-4′-(3,7-dimethylocta-2,6-dien-1-yl)-3′,5′-dihydroxy-[1,1′-biphenyl]-3,5-dicarboxylic acid, (E)-2-(3,7-dimethylocta-2,6-dien-1-yl)-5-(4-hydroxybutyl)benzene-1,3-diol, (E)-5-(4-aminobutyl)-2-(3,7-dimethylocta-2,6-dien-1-yl)benzene-1,3-diol, (E)-5-(4-(3,7-dimethylocta-2,6-dien-1-yl)-3,5-dihydroxyphenyl)pentanenitrile, (E)-2-(3,7-dimethylocta-2,6-dien-1-yl)-5-(3-methylhexan-2-yl)benzene-1,3-diol, (E)-3-(4-(3,7-dimethylocta-2,6-dien-1-yl)-3,5-dihydroxyphenyl)propanoic acid, (E)-2-(4-(3,7-dimethylocta-2,6-dien-1-yl)-3,5-dihydroxyphenyl)acetic acid, (E)-4-(4-(3,7-dimethylocta-2,6-dien-1-yl)-3,5-dihydroxyphenyl)butanoic acid, (E)-4-(3,7-dimethylocta-2,6-dien-1-yl)-3,5-dihydroxybenzoic acid, (E)-5-(4-(3,7-dimethylocta-2,6-dien-1-yl)-3,5-dihydroxyphenyl)pentanoic acid, (E)-6-(4-(3,7-dimethylocta-2,6-dien-1-yl)-3,5-dihydroxyphenyl)hexanoic acid, (E)-2-(3,7-dimethylocta-2,6-dien-1-yl)-5-pentylbenzene-1,3-diol, and (E)-2-(3,7-dimethylocta-2,6-dien-1-yl)-5-methylbenzene-1,3-diol.

Additional acidic cannabinoids and acidic cannabinoid derivatives of the methods or preparations of the present disclosure may also include, but are not limited to, 2,4-dihydroxy-3-[(1R,6R)-3-methyl-6-(prop-1-en-2-yl)cyclohex-2-en-1-yl]-6-propylbenzoic acid, 6-butyl-2,4-dihydroxy-3-[(1R,6R)-3-methyl-6-(prop-1-en-2-yl)cyclohex-2-en-1-yl]benzoic acid, 6-hexyl-2,4-dihydroxy-3-[(1R,6R)-3-methyl-6-(prop-1-en-2-yl)cyclohex-2-en-1-yl]benzoic acid, (1′R,2′R)-4-(hexan-2-yl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-(3-methylpentyl)-2′-(prop-1-en-2-yl)-1′,2′,3′,4′-tetrahydro-[1,1′-biphenyl]-3-carboxylic acid, (1′R,2′R)-4-(4-chlorobutyl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-(4-methylpentyl)-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-(4-(methylthio)butyl)-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-((E)-pent-1-en-1-yl)-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-((E)-pent-3-en-1-yl)-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-((E)-pent-2-en-1-yl)-2′-(prop-1-en-2-yl)-1′,2′,3′,4′-tetrahydro-[1,1′-biphenyl]-3-carboxylic acid, (1′R,2′R)-4-(but-3-yn-1-yl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-4-((E)-but-1-en-1-yl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-(pent-4-yn-1-yl)-2′-(prop-1-en-2-yl)-1′,2′,3′,4′-tetrahydro-[1,1′-biphenyl]-3-carboxylic acid, (1′R,2′R)-2,6-dihydroxy-5′-methyl-2′-(prop-1-en-2-yl)-4-undecyl-1′,2′,3′,4′-tetrahydro-[1,1′-biphenyl]-3-carboxylic acid, (1′R,2′R)-4-(hex-5-yn-1-yl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-4-((E)-hept-1-en-1-yl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-octyl-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-((E)-oct-1-en-1-yl)-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-nonyl-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-(3-phenylpropyl)-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-(4-phenylbutyl)-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-(5-phenylpentyl)-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-(6-phenylhexyl)-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-(2-methylpentyl)-2′-(prop-1-en-2-yl)-1′,2′,3′,4′-tetrahydro-[1,1′-biphenyl]-3-carboxylic acid, (1′R,2′R)-2,6-dihydroxy-4-isopropyl-5′-methyl-2′-(prop-1-en-2-yl)-1′,2′,3′,4′-tetrahydro-[1,1′-biphenyl]-3-carboxylic acid, (1′R,2′R)-4-decyl-2,6-dihydroxy-5′-methyl-2′-(prop-1-en-2-yl)-1′,2′,3′,4′-tetrahydro-[1,1′-biphenyl]-3-carboxylic acid, (1′R,2′R)-2,6-dihydroxy-5′-methyl-2′-(prop-1-en-2-yl)-4-tridecyl-1 ′,2′,3′,4′-tetrahydro-[1,1′-biphenyl]-3-carboxylic acid, (1′R,2′R)-4-((E)-2-carboxyvinyl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-4-((Z)-2-carboxyvinyl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-4-(6-carboxyhexyl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-4-(7-carboxyheptyl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-4-(8-carboxyoctyl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-4-(10-carboxydecyl)-2,6-dihydroxy-5′-methyl-2′-(prop-1-en-2-yl)-1′,2′,3′,4′-tetrahydro-[1,1′-biphenyl]-3-carboxylic acid, (1″R,2″R)-3′,5′-dihydroxy-5″-methyl-2″-(prop-1-en-2-yl)-1″,2″,3″,4″-tetrahydro-[1,1′:4′,1″-terphenyl]-2,2′-dicarboxylic acid, (1″R,2″R)-3′,5′-dihydroxy-5″-methyl-2″-(prop-1-en-2-yl)-1″,2″,3″,4″-tetrahydro-[1,1′:4′,1″-terphenyl]-2′,3-dicarboxylic acid, (1″R,2″R)-3′,5′-dihydroxy-5″-methyl-2″-(prop-1-en-2-yl)-1″,2″,3″,4″-tetrahydro-[1,1′:4′,1″-terphenyl]-2′,4-dicarboxylic acid, (1″R,2″R)-3′,5′-dihydroxy-5″-methyl-2″-(prop-1-en-2-yl)-1″,2″,3″,4″-tetrahydro-[1,1′:4′,1″-terphenyl]-2′,3,5-tricarboxylic acid, (1′R,2′R)-2,6-dihydroxy-4-(4-hydroxybutyl)-5′-methyl-2′-(prop-1-en-2-yl)-1′,2′,3′,4′-tetrahydro-[1,1′-biphenyl]-3-carboxylic acid, (1′R,2′R)-4-(4-aminobutyl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-4-(4-cyanobutyl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-(3-methylhexan-2-yl)-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-pentyl-2′-(prop-1-en-2-yl)-1′,2′,3′,4′-tetrahydro-[1,1′-biphenyl]-3-carboxylic acid, (1′R,2′R)-4-heptyl-2,6-dihydroxy-5′-methyl-2′-(prop-1-en-2-yl)-1′,2′,3′,4′-tetrahydro-[1,1′-biphenyl]-3-carboxylic acid, (1′R,2′R)-2,6-dihydroxy-5′-methyl-4-(pent-4-en-1-yl)-2′-(prop-1-en-2-yl)-1′,2′,3′,4′-tetrahydro-[1,1′-biphenyl]-3-carboxylic acid, (1′R,2′R)-4-(2-carboxyethyl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-2,6-dihydroxy-4,5′-dimethyl-2′-(prop-1-en-2-yl)-1′,2′,3′,4′-tetrahydro-[1,1′-biphenyl]-3-carboxylic acid, (1′R,2′R)-4-(carboxymethyl)-2,6-dihydroxy-5′-methyl-2′-(prop-1-en-2-yl)-1′,2′,3′,4′-tetrahydro-[1,1′-biphenyl]-3-carboxylic acid, (1′R,2′R)-4-(3-carboxypropyl)-2,6-dihydroxy-5′-methyl-2′-(prop-1-en-2-yl)-1′,2′,3′,4′-tetrahydro-[1,1′-biphenyl]-3-carboxylic acid,(1′R,2′R)-2,6-dihydroxy-5′-methyl-2′-(prop-1-en-2-yl)-1′,2′,3′,4′-tetrahydro-[1,1′-biphenyl]-3,4-dicarboxylic acid, (1′R,2′R)-4-(4-carboxybutyl)-2,6-dihydroxy-5′-methyl-2′-(prop-1-en-2-yl)-1′,2′,3′,4′-tetrahydro-[1,1′-biphenyl]-3-carboxylic acid, and (1′R,2′R)-4-(5-carboxypentyl)-2,6-dihydroxy-5′-methyl-2′-(prop-1-en-2-yl)-1′,2′,3′,4′-tetrahydro-[1,1′-biphenyl]-3-carboxylic acid.

Additional neutral cannabinoids and neutral cannabinoid derivatives of the methods or preparations of the present disclosure may also include, but are not limited to, (1′R,2′R)-4-(hexan-2-yl)-5′-methyl-2′-(prop-1-en-2-yl)-1′,2′,3′,4′-tetrahydro-[1,1′-biphenyl]-2,6-diol, (1′R,2′R)-4-hexyl-5′-methyl-2′-(prop-1-en-2-yl)-1′,2′,3′,4′-tetrahydro-[1,1′-biphenyl]-2,6-diol, (1′R,2′R)-5′-methyl-4-(3-methylpentyl)-2′-(prop-1-en-2-yl)-1′,2′,3′,4′-tetrahydro-[1,1′-biphenyl]-2,6-diol, (1′R,2′R)-4-(4-chlorobutyl)-5′-methyl-2′-(prop-1-en-2-yl)-1′,2′,3′,4′-tetrahydro-[1,1′-biphenyl]-2,6-diol, (1′R,2′R)-5′-methyl-4-(4-methylpentyl)-2′-(prop-1-en-2-yl)-1′,2′,3′,4′-tetrahydro-[1,1′-biphenyl]-2,6-diol, (1′R,2′R)-5′-methyl-4-(4-(methylthio)butyl)-2′-(prop-1-en-2-yl)-1′,2′,3′,4′-tetrahydro-[1,1′-biphenyl]-2,6-diol, (1′R,2′R)-5′-methyl-4-((E)-pent-1-en-1-yl)-2′-(prop-1-en-2-yl)-1′,2′,3′,4′-tetrahydro-[1,1′-biphenyl]-2,6-diol, (1′R,2′R)-5′-methyl-4-((E)-pent-3-en-1-yl)-2′-(prop-1-en-2-yl)-1′,2′,3′,4′-tetrahydro-[1,1′-biphenyl]-2,6-diol, (1′R,2′R)-5′-methyl-4-((E)-pent-2-en-1-yl)-2′-(prop-1-en-2-yl)-1′,2′,3′,4′-tetrahydro-[1,1′-biphenyl]-2,6-diol, (1′R,2′R)-4-(but-3-yn-1-yl)-5′-methyl-2′-(prop-1-en-2-yl)-1′,2′,3′,4′-tetrahydro-[1,1′-biphenyl]-2,6-diol, (1′R,2′R)-4-((E)-but-1-en-1-yl)-5′-methyl-2′-(prop-1-en-2-yl)-1′,2′,3′,4′-tetrahydro-[1,1′-biphenyl]-2,6-diol, (1′R,2′R)-5′-methyl-4-(pent-4-yn-1-yl)-2′-(prop-1-en-2-yl)-1′,2′,3′,4′-tetrahydro-[1,1′-biphenyl]-2,6-diol, (1′R,2′R)-5′-methyl-2′-(prop-1-en-2-yl)-4-undecyl-1′,2′,3′,4′-tetrahydro-[1,1′-biphenyl]-2,6-diol, (1′R,2′R)-4-(hex-5-yn-1-yl)-5′-methyl-2′-(prop-1-en-2-yl)-1′,2′,3′,4′-tetrahydro-[1,1′-biphenyl]-2,6-diol, (1′R,2′R)-4-((E)-hept-1-en-1-yl)-5′-methyl-2′-(prop-1-en-2-yl)-1′,2′,3′,4′-tetrahydro-[1,1′-biphenyl]-2,6-diol, (1′R,2′R)-5′-methyl-4-octyl-2′-(prop-1-en-2-yl)-1′,2′,3′,4′-tetrahydro-[1,1′-biphenyl]-2,6-diol, (1′R,2′R)-5′-methyl-4-((E)-oct-1-en-1-yl)-2′-(prop-1-en-2-yl)-1′,2′,3′,4′-tetrahydro-[1,1′-biphenyl]-2,6-diol, (1′R,2′R)-5′-methyl-4-nonyl-2′-(prop-1-en-2-yl)-1′,2′,3′,4′-tetrahydro-[1,1′-biphenyl]-2,6-diol, (1′R,2′R)-5′-methyl-4-(3-phenylpropyl)-2′-(prop-1-en-2-yl)-1′,2′,3′,4′-tetrahydro-[1,1′-biphenyl]-2,6-diol, (1′R,2′R)-5′-methyl-4-(4-phenylbutyl)-2′-(prop-1-en-2-yl)-1′,2′,3′,4′-tetrahydro-[1,1′-biphenyl]-2,6-diol, (1′R,2′R)-5′-methyl-4-(5-phenylpentyl)-2′-(prop-1-en-2-yl)-1′,2′,3′,4′-tetrahydro-[1,1′-biphenyl]-2,6-diol, (1′R,2′R)-5′-methyl-4-(6-phenylhexyl)-2′-(prop-1-en-2-yl)-1′,2′,3′,4′-tetrahydro-[1,1′-biphenyl]-2,6-diol, (1′R,2′R)-5′-methyl-4-(2-methylpentyl)-2′-(prop-1-en-2-yl)-1′,2′,3′,4′-tetrahydro-[1,1′-biphenyl]-2,6-diol, (1′R,2′R)-4-isopropyl-5′-methyl-2′-(prop-1-en-2-yl)-1′,2′,3′,4′-tetrahydro-[1,1′-biphenyl]-2,6-diol, (1′R,2′R)-4-decyl-5′-methyl-2′-(prop-1-en-2-yl)-1′,2′,3′,4′-tetrahydro-[1,1′-biphenyl]-2,6-diol, (1′R,2′R)-5′-methyl-2′-(prop-1-en-2-yl)-4-tridecyl-1′,2′,3′,4′-tetrahydro-[1,1′-biphenyl]-2,6-diol, (E)-3-((1′R,2′R)-2,6-dihydroxy-5′-methyl-2′-(prop-1-en-2-yl)-1′,2′,3′,4′-tetrahydro-[1,1′-biphenyl]-4-yl)acrylic acid, (Z)-3-((1′R,2′R)-2,6-dihydroxy-5′-methyl-2′-(prop-1-en-2-yl)-1′,2′,3′,4′-tetrahydro-[1,1′-biphenyl]-4-yl)acrylic acid, 7-((1′R,2′R)-2,6-dihydroxy-5′-methyl-2′-(prop-1-en-2-yl)-1′,2′,3′,4′-tetrahydro-[1,1′-biphenyl]-4-yl)heptanoic acid, 8-((1′R,2′R)-2,6-dihydroxy-5′-methyl-2′-(prop-1-en-2-yl)-1′,2′,3′,4′-tetrahydro-[1,1′-biphenyl]-4-yl)octanoic acid, 9(1′R,2′R)-2,6-dihydroxy-5′-methyl-2′-(prop-1-en-2-yl)-1′,2′,3′,4′-tetrahydro-[1,1′-biphenyl]-4-yl)nonanoic acid, 11-((1′R,2′R)-2,6-dihydroxy-5′-methyl-2′-(prop-1-en-2-yl)-1′,2′,3′,4′-tetrahydro-[1,1′-biphenyl]-4-yl)undecanoic acid, (1″R,2″R)-3′,5′-dihydroxy-5″-methyl-2″-(prop-1-en-2-yl)-1″,2″,3″,4″-tetrahydro-[1,1′:4′,1″-terphenyl]-2-carboxylic acid, (1″R,2″R)-3′,5′-dihydroxy-5″-methyl-2″-(prop-1-en-2-yl)-1″,2″,3″,4″-tetrahydro-[1,1′:4′,1″-terphenyl]-3-carboxylic acid, (1″R,2″R)-3′,5′-dihydroxy-5″-methyl-2″-(prop-1-en-2-yl)-1″,2″,3″,4″-tetrahydro-[1,1′:4′,1″-terphenyl]-4-carboxylic acid, (1″R,2″R)-3′,5′-dihydroxy-5″-methyl-2″-(prop-1-en-2-yl)-1″,2″,3″,4″-tetrahydro-[1,1′:4′,1″-terphenyl]-3,5-dicarboxylic acid, (1′R,2′R)-4-(4-hydroxybutyl)-5′-methyl-2′-(prop-1-en-2-yl)-1′,2′,3′,4′-tetrahydro-[1,1′-biphenyl]-2,6-diol, (1′R,2′R)-4-(4-aminobutyl)-5′-methyl-2′-(prop-1-en-2-yl)-1′,2′,3′,4′-tetrahydro-[1,1′-biphenyl]-2,6-diol, 5-((1′R,2′R)-2,6-dihydroxy-5′-methyl-2′-(prop-1-en-2-yl)-1′,2′,3′,4′-tetrahydro-[1,1′-biphenyl]-4-yl)pentanenitrile, (1′R,2′R)-5′-methyl-4-(3-methylhexan-2-yl)-2′-(prop-1-en-2-yl)-1′,2′,3′,4′-tetrahydro-[1,1′-biphenyl]-2,6-diol, (1′R,2′R)-5′-methyl-2′-(prop-1-en-2-yl)-4-propyl-1′,2′,3′,4′-tetrahydro-[1,1′-biphenyl]-2,6-diol, (1′R,2′R)-4-butyl-5′-methyl-2′-(prop-1-en-2-yl)-1′,2′,3′,4′-tetrahydro-[1,1′-biphenyl]-2,6-diol, (1′R,2′R)-5′-methyl-4-pentyl-2′-(prop-1-en-2-yl)-1′,2′,3′,4′-tetrahydro-[1,1′-biphenyl]-2,6-diol, (1′R,2′R)-4-heptyl-5′-methyl-2′-(prop-1-en-2-yl)-1′,2′,3′,4′-tetrahydro-[1,1′-biphenyl]-2,6-diol, (1′R,2′R)-5′-methyl-4-(pent-4-en-1-yl)-2′-(prop-1-en-2-yl)-1′,2′,3′,4′-tetrahydro-[1,1′-biphenyl]-2,6-diol, 3-((1′R,2′R)-2,6-dihydroxy-5′-methyl-2′-(prop-1-en-2-yl)-1′,2′,3′,4′-tetrahydro-[1,1′-biphenyl]-4-yl)propanoic acid, (1′R,2′R)-4,5′-dimethyl-2′-(prop-1-en-2-yl)-1′,2′,3′,4′-tetrahydro-[1,1′-biphenyl]-2,6-diol, 2-((1′R,2′R)-2,6-dihydroxy-5′-methyl-2′-(prop-1-en-2-yl)-1′,2′,3′,4′-tetrahydro-[1,1′-biphenyl]-4-yl)acetic acid, 4-((1′R,2′R)-2,6-dihydroxy-5′-methyl-2′-(prop-1-en-2-yl)-1′,2′,3′,4′-tetrahydro-[1,1′-biphenyl]-4-yl)butanoic acid, (1′R,2′R)-2,6-dihydroxy-5′-methyl-2′-(prop-1-en-2-yl)-1′,2′,3′,4′-tetrahydro-[1,1′-biphenyl]-4-carboxylic acid, 5-((1′R,2′R)-2,6-dihydroxy-5′-methyl-2′-(prop-1-en-2-yl)-1′,2′,3′,4′-tetrahydro-[1,1′-biphenyl]-4-yl)pentanoic acid, and 6-((1′R,2′R)-2,6-dihydroxy-5′-methyl-2′-(prop-1-en-2-yl)-1′,2′,3′,4′-tetrahydro-[1,1′-biphenyl]-4-yl)hexanoic acid.

In some embodiments, the neutral cannabinoids or neutral cannabinoid derivatives of the methods or preparations of the present disclosure may also include compounds of Formula (I):

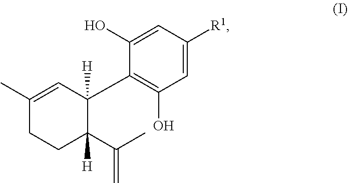

wherein,

R¹ is a C₁-C₁₈alkyl group substituted with R^{a} or R¹ is a C₂-C₁₈alkenyl group substituted with R^{a}, wherein R^{a} is —S—C₁-C₆alkyl or —S—C₂-C₆alkenyl, or R¹ is selected from the group consisting of:

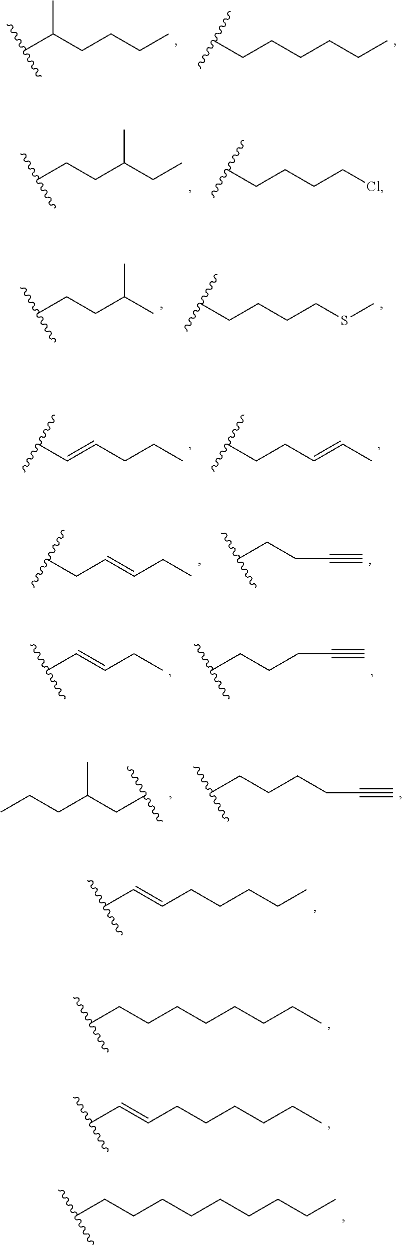

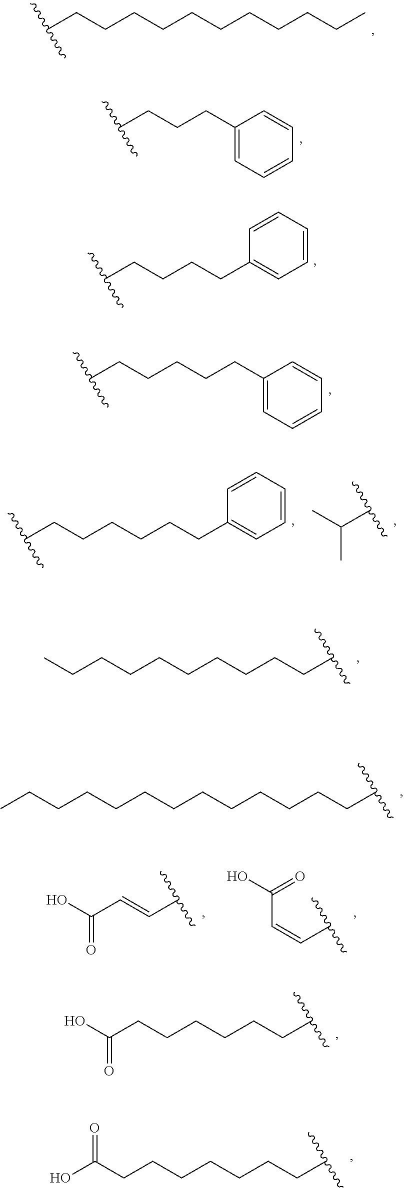

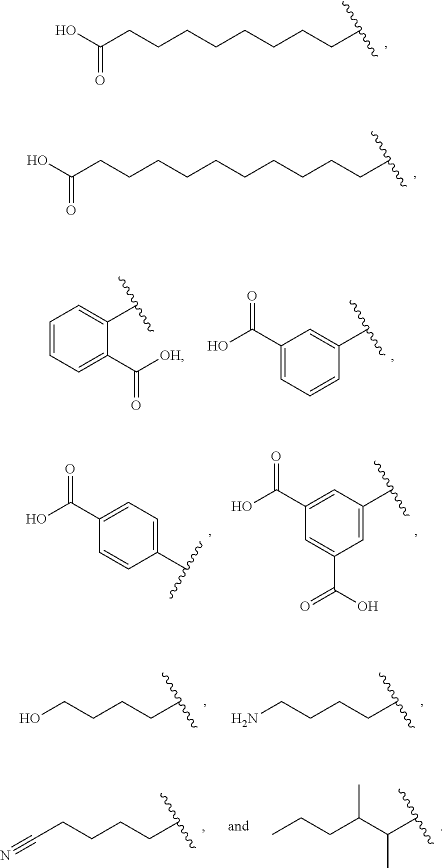

In some embodiments, the compound of Formula (I) may include where R¹ is a C₁-C₁₈alkyl group substituted with R^{a} or R¹ is a C₂-C₁₈alkenyl group substituted with R^{a}, wherein R^{a} is —S—C₁-C₆alkyl or —S—C₂-C₆alkenyl.

In some embodiments, the compound of Formula (I) is selected from the group consisting of:

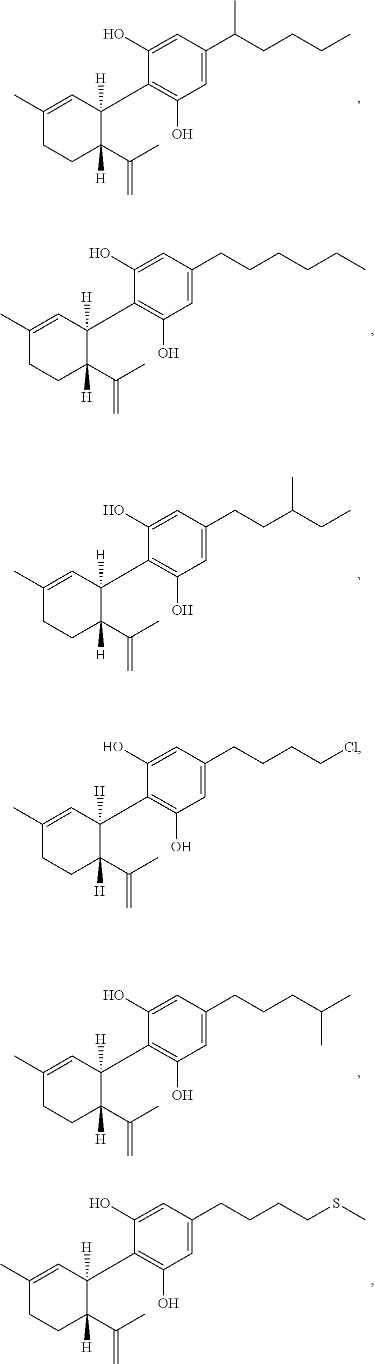

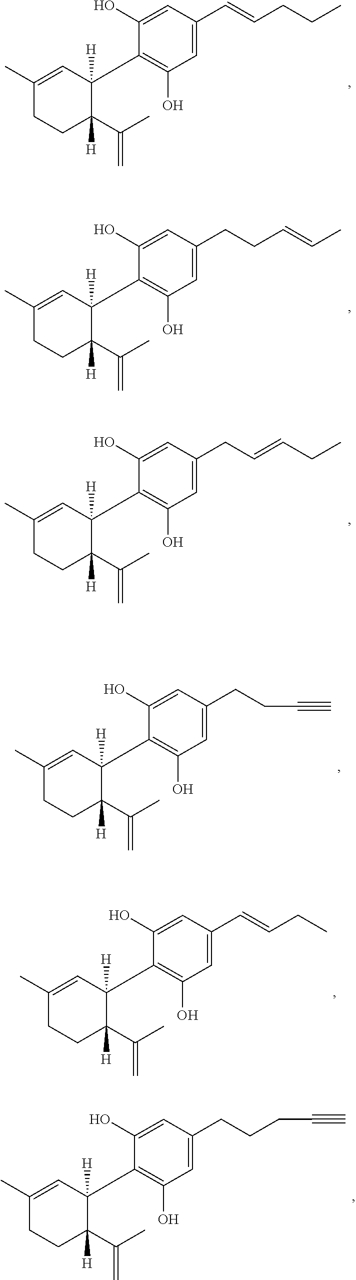

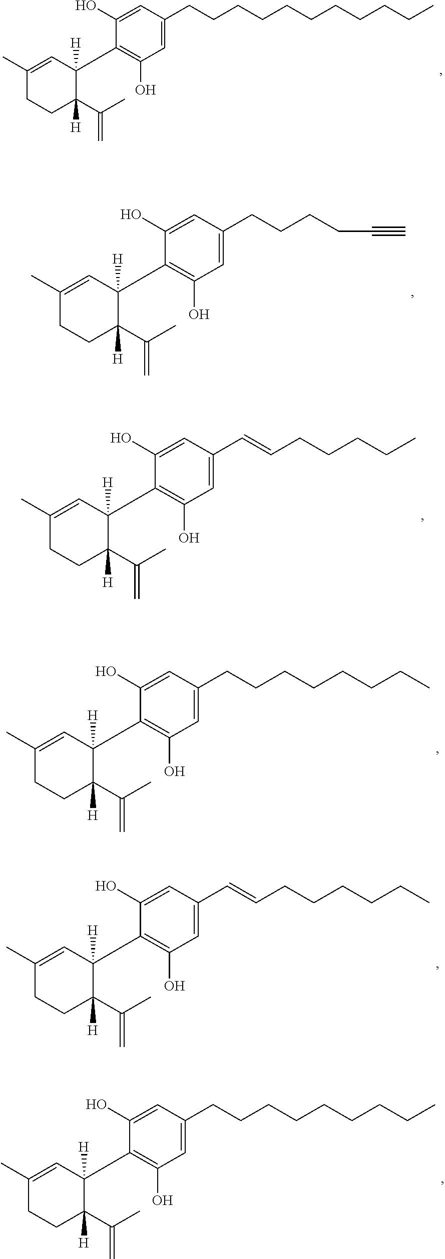

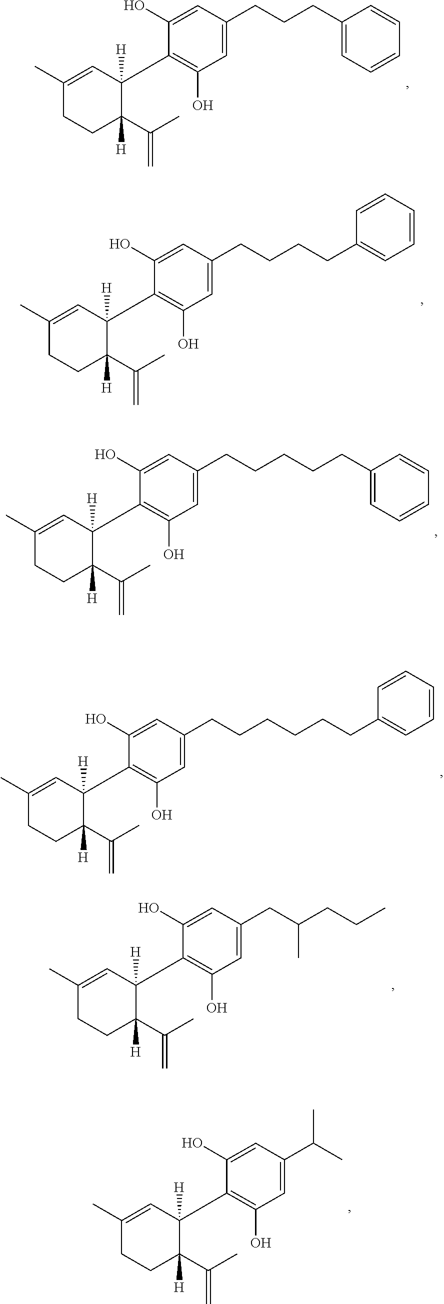

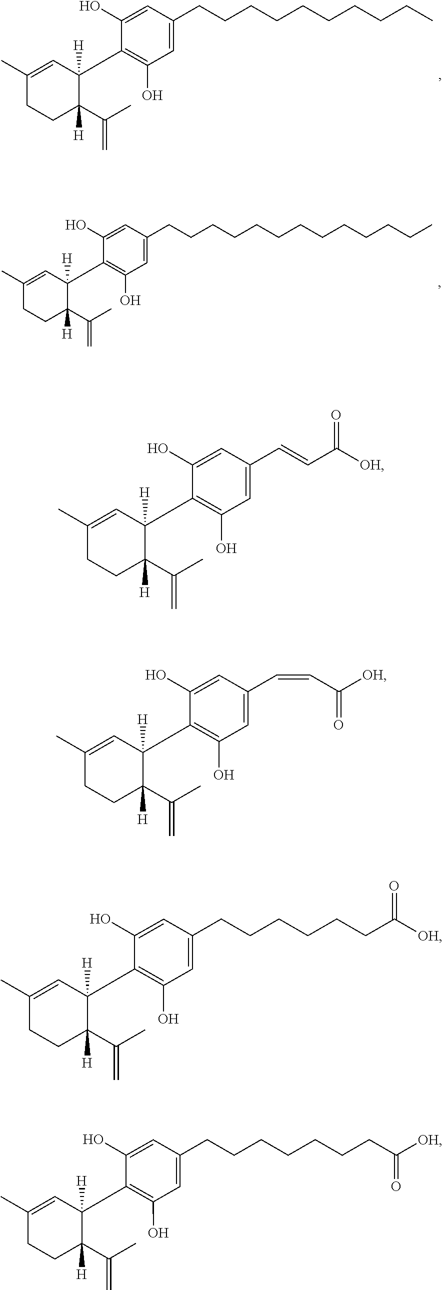

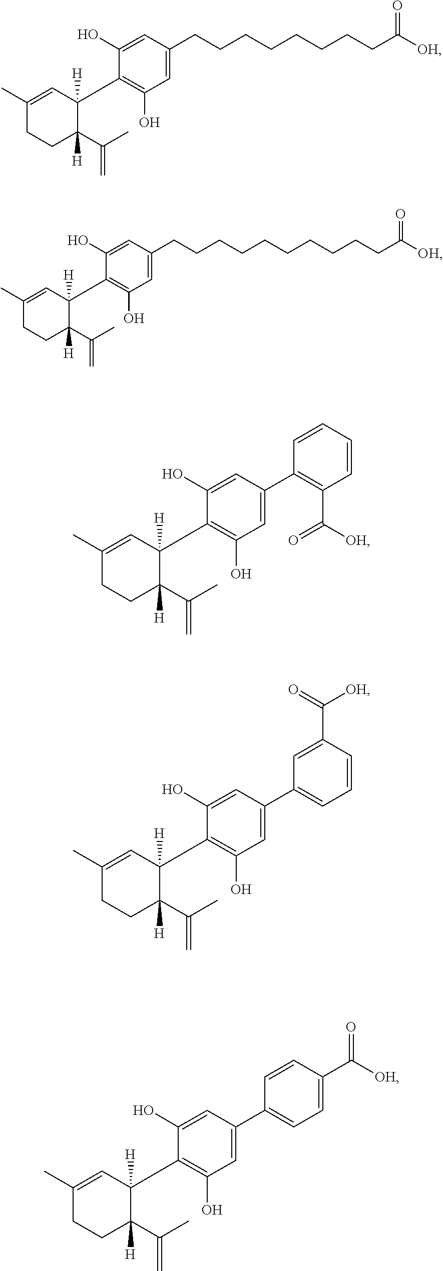 [SMILES: C=C(C)[C@@H]1CCC(C)=C[C@H]1c1c(O)cc(CCCCCCCCC(=O)O)cc1O ; C=C(C)[C@@H]1CCC(C)=C[C@H]1c1c(O)cc(CCCCCCCCCCC(=O)O)cc1O ; C=C(C)[C@@H]1CCC(C)=C[C@H]1c1c(O)cc(-c2ccccc2C(=O)O)cc1O ; C=C(C)[C@@H]1CCC(C)=C[C@H]1c1c(O)cc(-c2cccc(C(=O)O)c2)cc1O ; C=C(C)[C@@H]1CCC(C)=C[C@H]1c1c(O)cc(-c2ccc(C(=O)O)cc2)cc1O]

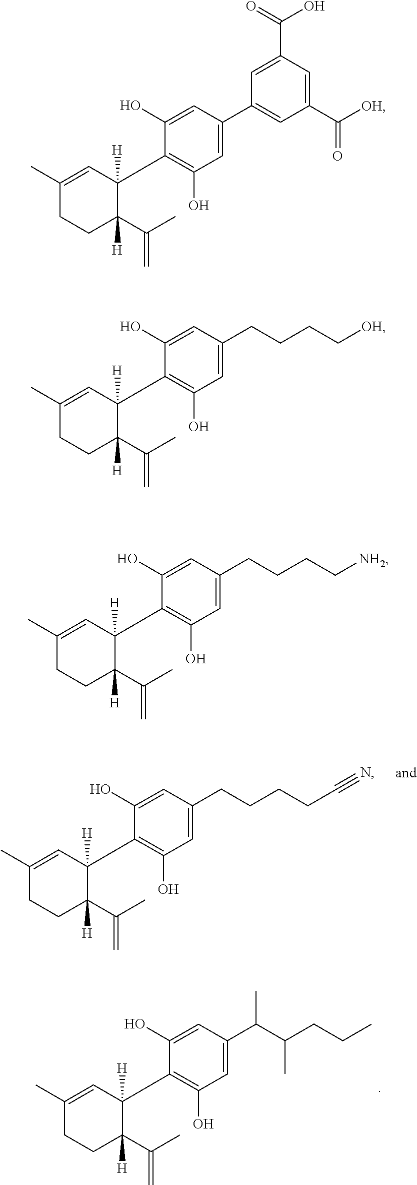 [SMILES: C=C(C)[C@@H]1CCC(C)=C[C@H]1c1c(O)cc(-c2cc(C(=O)O)cc(C(=O)O)c2)cc1O ; C=C(C)[C@@H]1CCC(C)=C[C@H]1c1c(O)cc(CCCCO)cc1O ; C=C(C)[C@@H]1CCC(C)=C[C@H]1c1c(O)cc(CCCCN)cc1O ; C=C(C)[C@@H]1CCC(C)=C[C@H]1c1c(O)cc(CCCCC#N)cc1O ; C=C(C)[C@@H]1CCC(C)=C[C@H]1c1c(O)cc(C(C)C(C)CCC)cc1O]

A cannabinoid derivative may also refer to a compound lacking one or more chemical moieties found in naturally-occurring cannabinoids, yet retains the core structural features (e.g., cyclic core) of a naturally-occurring cannabinoid. Such chemical moieties may include, but are not limited to, methyl, alkyl, alkenyl, methoxy, alkoxy, acetyl, carboxyl, carbonyl, oxo, ester, hydroxyl, and the like. In some embodiments, a cannabinoid derivative may also comprise one or more of any of the functional and/or reactive groups described herein. Functional and reactive groups may be unsubstituted or substituted with one or more functional or reactive groups.

A cannabinoid derivative may be a cannabinoid substituted with or comprising one or more functional and/or reactive groups. Functional groups may include, but are not limited to, azido, halo (e.g., chloride, bromide, iodide, fluorine), methyl, alkyl, alkynyl, alkenyl, methoxy, alkoxy, acetyl, amino, carboxyl, carbonyl, oxo, ester, hydroxyl, thio (e.g., thiol), cyano, aryl, heteroaryl, cycloalkyl, cycloalkenyl, cycloalkylalkenyl, cycloalkylalkynyl, cycloalkenylalkyl, cycloalkenylalkenyl, cycloalkenylalkynyl, heterocyclylalkenyl, heterocyclylalkynyl, heteroarylalkenyl, heteroarylalkynyl, arylalkenyl, arylalkynyl, spirocyclyl, heterospirocyclyl, heterocyclyl, thioalkyl (or alkylthio), arylthio, heteroarylthio, sulfone, sulfonyl, sulfoxide, amido, alkylamino, dialkylamino, arylamino, alkylarylamino, diarylamino, N-oxide, imide, enamine, imine, oxime, hydrazone, nitrile, aralkyl, cycloalkylalkyl, haloalkyl, heterocyclylalkyl, heteroarylalkyl, nitro, thioxo, and the like. Suitable reactive groups may include, but are not necessarily limited to, azide, carboxyl, carbonyl, amine (e.g., alkyl amine (e.g., lower alkyl amine), aryl amine), halide, ester (e.g., alkyl ester (e.g., lower alkyl ester, benzyl ester), aryl ester, substituted aryl ester), cyano, thioester, thioether, sulfonyl halide, alcohol, thiol, succinimidyl ester, isothiocyanate, iodoacetamide, maleimide, hydrazine, alkynyl, alkenyl, acetyl, and the like. In some embodiments, the reactive group is selected from a carboxyl, a carbonyl, an amine, an ester, a thioester, a thioether, a sulfonyl halide, an alcohol, a thiol, an alkyne, alkene, an azide, a succinimidyl ester, an isothiocyanate, an iodoacetamide, a maleimide, and a hydrazine. Functional and reactive groups may be unsubstituted or substituted with one or more functional or reactive groups.

“Alkyl” may refer to a straight or branched chain saturated hydrocarbon. For example, C₁-C₆alkyl groups contain 1 to 6 carbon atoms. Examples of a C₁-C₆alkyl group include, but are not limited to, methyl, ethyl, propyl, butyl, pentyl, isopropyl, isobutyl, sec-butyl and tent-butyl, isopentyl, and neopentyl.

“Alkenyl” may include an unbranched (i.e., straight) or branched hydrocarbon chain containing 2-12 carbon atoms. The “alkenyl” group contains at least one double bond. The double bond of an alkenyl group can be unconjugated or conjugated to another unsaturated group. Examples of alkenyl groups may include, but are not limited to, ethylenyl, vinyl, allyl, butenyl, pentenyl, hexenyl, butadienyl, pentadienyl, hexadienyl, 2-ethylhexenyl, 2-propyl-2-butenyl, 4-(2-methyl-3-butene)-pentenyl and the like.

Compounds disclosed herein, such as cannabinoids and cannabinoid derivatives, may be substituted with one or more substituents, such as those illustrated generally herein, or as exemplified by particular classes, subclasses, and species of the present disclosure. In general, the term “substituted” refers to the replacement of a hydrogen atom in a given structure with a specified sub stituent. Combinations of sub stituents envisioned by the present disclosure are typically those that result in the formation of stable or chemically feasible compounds.

As used herein, the term “unsubstituted” may mean that the specified group bears no substituents beyond the moiety recited (e.g., where valency satisfied by hydrogen).

A reactive group may facilitate covalent attachment of a molecule of interest. Suitable molecules of interest may include, but are not limited to, a detectable label; imaging agents; a toxin (including cytotoxins); a linker; a peptide; a drug (e.g., small molecule drugs); a member of a specific binding pair; an epitope tag; ligands for binding by a target receptor; tags to aid in purification; molecules that increase solubility; and the like. A linker may be a peptide linker or a non-peptide linker.

In some embodiments, a cannabinoid derivative substituted with an azide may be reacted with a compound comprising an alkyne group via “click chemistry” to generate a product comprising a heterocycle, also known as an azide-alkyne cycloaddition. In some embodiments, a cannabinoid derivative substituted with an alkyne may be reacted with a compound comprising an azide group via click chemistry to generate a product comprising a heterocycle.

Additional molecules of interest that may be desirable for attachment to a cannabinoid derivative may include, but are not necessarily limited to, detectable labels (e.g., spin labels, fluorescence resonance energy transfer (FRET)-type dyes, e.g., for studying structure of biomolecules in vivo); small molecule drugs; cytotoxic molecules (e.g., drugs); imaging agents; ligands for binding by a target receptor; tags to aid in purification by, for example, affinity chromatography (e.g., attachment of a FLAG epitope); molecules that increase solubility (e.g., poly(ethylene glycol)); molecules that enhance bioavailability; molecules that increase in vivo half-life; molecules that target to a particular cell type (e.g., an antibody specific for an epitope on a target cell); molecules that target to a particular tissue; molecules that provide for crossing the blood-brain barrier; and molecules to facilitate selective attachment to a surface, and the like.

In some embodiments, a molecule of interest comprises an imaging agent. Suitable imaging agents may include positive contrast agents and negative contrast agents. Suitable positive contrast agents may include, but are not limited to, gadolinium tetraazacyclododecanetetraacetic acid (Gd-DOTA); gadolinium-diethylenetriaminepentaacetic acid (Gd-DTPA); gadolinium-1,4,7-tris(carbonylmethyl)-10-(2′-hydroxypropyl)-1,4,7,10-tetraazacyclododecane (Gd-HP-DO3A); Manganese(II)-dipyridoxal diphosphate (Mn-DPDP); Gd-diethylenetriaminepentaacetate-bis(methylamide) (Gd-DTPA-BMA); and the like. Suitable negative contrast agents may include, but are not limited to, a superparamagnetic iron oxide (SPIO) imaging agent; and a perfluorocarbon, where suitable perfluorocarbons may include, but are not limited to, fluoroheptanes, fluorocycloheptanes, fluoromethylcycloheptanes, fluorohexanes, fluorocyclohexanes, fluoropentanes, fluorocyclopentanes, fluoromethylcyclopentanes, fluorodimethylcyclopentanes, fluoromethylcyclobutanes, fluorodimethylcyclobutanes, fluorotrimethylcyclobutanes, fluorobutanes, fluorocyclobutanes, fluoropropanes, fluoroethers, fluoropolyethers, fluorotriethylamines, perfluorohexanes, perfluoropentanes, perfluorobutanes, perfluoropropanes, sulfur hexafluoride, and the like.

Additional cannabinoid derivatives of the methods or cannabinoid derivative preparations of the disclosure may include derivatives that have been modified via organic synthesis or an enzymatic route to modify drug metabolism and pharmacokinetics (e.g., solubility, bioavailability, absorption, distribution, plasma half-life and metabolic clearance). Modification examples may include, but are not limited to, halogenation, acetylation, and methylation.

The cannabinoids or cannabinoid derivatives of the methods or cannabinoid or cannabinoid derivative preparations of the disclosure further include all pharmaceutically acceptable isotopically labeled versions of the cannabinoids or cannabinoid derivatives. An “isotopically-” or “radio-labeled” compound is a compound where one or more atoms are replaced or substituted by an atom having an atomic mass or mass number different from the atomic mass or mass number typically found in nature (i.e., naturally occurring). For example, in some embodiments, in the cannabinoids or cannabinoid derivatives of the methods or cannabinoid or cannabinoid derivative preparations of the disclosure, hydrogen atoms are replaced or substituted by one or more deuterium or tritium. Certain isotopically labeled cannabinoids or cannabinoid derivatives of the methods or cannabinoid or cannabinoid derivative preparations of the disclosure, for example, those incorporating a radioactive isotope, are useful in drug and/or substrate tissue distribution studies. The radioactive isotopes tritium, i.e., ³H, and carbon 14, i.e., ¹⁴C, are particularly useful for this purpose in view of their ease of incorporation and ready means of detection. Substitution with heavier isotopes such as deuterium, i.e., ²H, may afford certain therapeutic advantages resulting from greater metabolic stability, for example, increased in vivo half-life or reduced dosage requirements, and hence may be preferred in some circumstances. Suitable isotopes that may be incorporated in cannabinoids or cannabinoid derivatives of the methods or cannabinoid or cannabinoid derivative preparations of the disclosure include but are not limited to ²H (also written as D for deuterium)³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I , ¹²⁴I, ¹²⁵I, and ¹³¹I. Substitution with positron emitting isotopes, such as ¹¹C, ¹⁸F, ¹⁵O, and ¹³N, can be useful in Positron Emission Topography (PET) studies.

Methods of bioproduction enable synthesis of cannabinoids or cannabinoid derivatives with defined stereochemistries, which is challenging to do using chemical synthesis. Cannabinoids or cannabinoid derivatives of the methods or preparations of the disclosure may be enantiomers or disastereomers. The term “enantiomers” may refer to a pair of stereoisomers which are non-superimposable mirror images of one another. In some embodiments the cannabinoids or cannabinoid derivatives may be the (S)-enantiomer. In some embodiments the cannabinoids or cannabinoid derivatives may be the (R)-enantiomer. In some embodiments, the cannabinoids or cannabinoid derivatives may be the (+) or (-) enantiomers. The term “diastereomers” may refer to the set of stereoisomers which cannot be made superimposable by rotation around single bonds. For example, cis- and trans-double bonds, endo- and exo-substitution on bicyclic ring systems, and compounds containing multiple stereogenic centers with different relative configurations may be considered to be diastereomers. The term “diastereomer” may refer to any member of this set of compounds. Cannabinoids or cannabinoid derivatives of the methods or preparations of the disclosure may include a double bond or a fused ring. In certain such embodiments, the double bond or fused ring may be cis or trans, unless the configuration is specifically defined. If the cannabinoid or cannabinoid derivative contains a double bond, the substituent may be in the E or Z configuration, unless the configuration is specifically defined.

In some embodiments, the cannabinoid or cannabinoid derivative of the methods or preparations of the disclosure is in the form of a salt. In certain such embodiments, the salt is a pharmaceutically acceptable salt. In some embodiments, the methods or cannabinoid or cannabinoid derivative preparations disclosed herein may comprise a salt of the cannabinoid or cannabinoid derivative. In certain such embodiments, the salt is a pharmaceutically acceptable salt. In some embodiments, the salt is an acceptable salt. In some embodiments, the cannabinoid or cannabinoid derivative recovered in the methods or preparations of the disclosure is not in the form of a salt.

“Pharmaceutically acceptable salts” or “acceptable salts” may refer to those salts which retain the biological effectiveness and properties of the free bases, which are not biologically or otherwise undesirable. Representative pharmaceutically acceptable salts or acceptable salts include, but are not limited to, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium, calcium edetate, camsylate, carbonate, chloride, citrate, clavulariate, dihydrochloride, edetate, edisylate, estolate, esylate, fiunarate, gluceptate, gluconate, glutamate, glycollylarsanilate, hexafluorophosphate, hexylresorcinate, hydrabamine, hydrobromide, hydrochloride, hydroxynaphthoate, iodide, sethionate, lactate, lactobionate, laurate, magnesium, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosalicylate, suramate, tannate, tartrate, teoclate, tosylate, triethiodide, and valerate salts.

“Acceptable salt” or “pharmaceutically acceptable salt” also includes both acid and base addition salts. “Acceptable acid addition salt” or “pharmaceutically acceptable acid addition salt” may refer to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Acceptable base addition salt” or “pharmaceutically acceptable base addition salt” may refer to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. For example, inorganic salts include, but are not limited to, ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like.

### Preparation of Cannabinoids or Cannabinoid Derivative Using Modified Host Cells

### Cannabinoid Biosynthesis

The methods and preparations disclosed herein comprise cannabinoids and/or cannabinoid derivatives (e.g., acidic cannabinoids and/or acidic cannabinoid derivatives or neutral cannabinoids and/or neutral cannabinoid derivatives) produced using modified host cells. These modified host cells comprise one or more nucleic acids (e.g., heterologous nucleic acids) encoding one or more polypeptides having at least one activity of a polypeptide present in the cannabinoid or cannabinoid precursor (e.g., geranylpyrophosphate (GPP), prenyl phosphates, olivetolic acid, or hexanoyl-CoA) biosynthetic pathway. Exemplary modified host cells are detailed in WO2018/200888 filed Apr. 27, 2018 and WO 2020/069214 filed Sep. 26, 2019, and PCT/US2020/051261 filed Sep. 17, 2020. Accordingly, the modified host cells described herein may refer to the modified host cells detailed in WO2018/200888 filed Apr. 27, 2018 and WO 2020/069214 filed Sep. 26, 2019, and PCT/US2020/051261 filed Sep. 17, 2020.

As detailed in WO2018/200888 filed Apr. 27, 2018 and WO 2020/069214 filed Sep. 26, 2019, and PCT/US2020/051261 filed Sep. 17, 2020, in Cannabis, cannabinoids are produced from the common metabolite precursors geranylpyrophosphate (GPP) and hexanoyl-CoA by the action of three polypeptides. Hexanoyl-CoA and malonyl-CoA are combined to afford a 12-carbon tetraketide intermediate by a tetraketide synthase (TKS) polypeptide. This tetraketide intermediate is then cyclized by an olivetolic acid cyclase (OAC) polypeptide to produce olivetolic acid. Olivetolic acid is then prenylated with the common isoprenoid precursor GPP by a geranyl pyrophosphate:olivetolic acid geranyltransferase (GOT) polypeptide to produce cannabigerolic acid (CBGA), the cannabinoid also known as the “mother cannabinoid.” Different cannabinoid synthase polypeptides then convert CBGA into other cannabinoids, e.g., a tetrahydrocannabinolic acid (THCA) synthase polypeptide produces THCA, a CBDA synthase polypeptide produces CBDA, etc. In the presence of heat or light, the acidic cannabinoids can undergo decarboxylation, e.g., THCA producing THC or CBDA producing CBD.

GPP and hexanoyl-CoA can be generated through several pathways. One or more nucleic acids (e.g., heterologous nucleic acids) encoding one or more polypeptides having at least one activity of a polypeptide present in these pathways may be present in the modified host cells used herein. Such modified host cells are detailed in WO2018/200888, filed Apr. 27, 2018, and WO 2020/069214, filed Sep. 26, 2019, and PCT/US2020/051261 filed Sep. 17, 2020.

Polypeptides that generate GPP or are part of a biosynthetic pathway that generates GPP may be one or more polypeptides having at least one activity of a polypeptide present in the mevalonate (MEV) pathway (e.g., one or more MEV pathway polypeptides). The term “mevalonate pathway” or “MEV pathway,” as used herein, may refer to the biosynthetic pathway that converts acetyl-CoA to isopentenyl pyrophosphate (IPP) and dimethylallyl pyrophosphate (DMAPP). The mevalonate pathway comprises polypeptides that catalyze the following steps: (a) condensing two molecules of acetyl-CoA to generate acetoacetyl-CoA (e.g., by action of an acetoacetyl-CoA thiolase polypeptide); (b) condensing acetoacetyl-CoA with acetyl-CoA to form hydroxymethylglutaryl-CoA (HMG-CoA) (e.g., by action of a HMG-CoA synthase (HMGS) polypeptide); (c) converting HMG-CoA to mevalonate (e.g., by action of a HMG-CoA reductase (HMGR) polypeptide); (d) phosphorylating mevalonate to mevalonate 5-phosphate (e.g., by action of a mevalonate kinase (MK) polypeptide); (e) converting mevalonate 5-phosphate to mevalonate 5-pyrophosphate (e.g., by action of a phosphomevalonate kinase (PMK) polypeptide); (f) converting mevalonate 5-pyrophosphate to isopentenyl pyrophosphate (e.g., by action of a mevalonate pyrophosphate decarboxylase (MVD1) polypeptide); and (g) converting isopentenyl pyrophosphate (IPP) to dimethylallyl pyrophosphate (DMAPP) (e.g., by action of an isopentenyl pyrophosphate isomerase (IDI1) polypeptide). A geranyl pyrophosphate synthetase (GPPS) polypeptide then acts on IPP and/or DMAPP to generate GPP. Polypeptides that generate hexanoyl-CoA may include polypeptides that generate acyl-CoA compounds or acyl-CoA compound derivatives (e.g., a hexanoyl-CoA synthase (HCS) polypeptide, an acyl-activating enzyme polypeptide, a fatty acyl-CoA synthetase polypeptide, or a fatty acyl-CoA ligase polypeptide). Hexanoyl CoA derivatives, acyl-CoA compounds, or acyl-CoA compound derivatives may also be formed via such polypeptides.

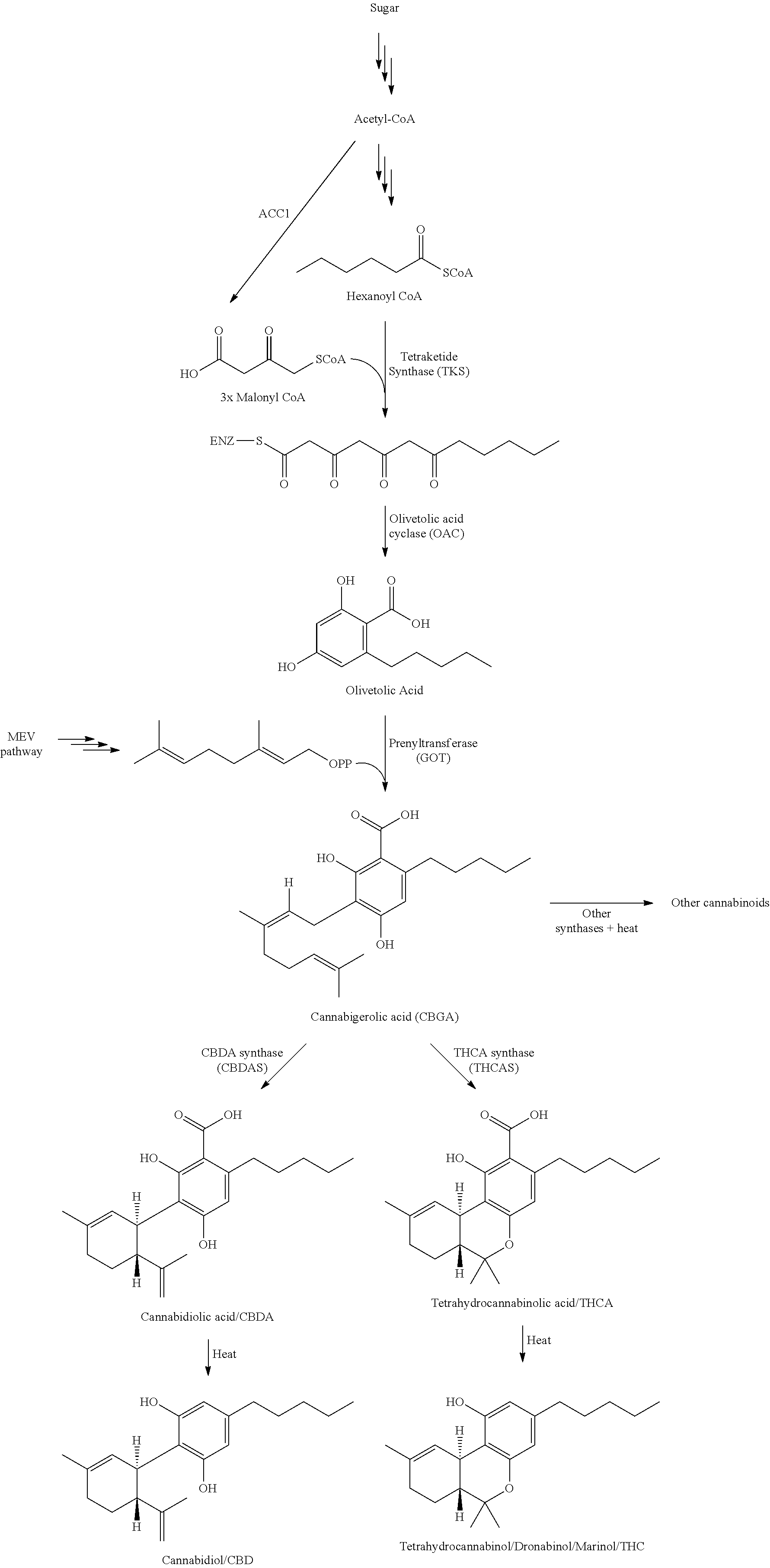

### Biosynthetic Pathways to Cannabinoids

GPP and hexanoyl-CoA may also be generated through pathways comprising polypeptides that condense two molecules of acetyl-CoA to generate acetoacetyl-CoA and pyruvate decarboxylase polypeptides that generate acetyl-CoA from pyruvate via acetaldehyde. Hexanoyl CoA derivatives, acyl-CoA compounds, or acyl-CoA compound derivatives may also be formed via such pathways.

### Modified Host Cells

The methods of the disclosure may include using microorganisms engineered (e.g., modified host cells) to produce naturally-occurring and non-naturally occurring cannabinoids. Naturally-occurring cannabinoids and non-naturally occurring cannabinoids (e.g., cannabinoid derivatives) are challenging to produce using chemical synthesis due to their complex structures. However, as detailed in WO2018/200888 filed Apr. 27, 2018 and WO 2020/069214 filed Sep. 26, 2019, and PCT/US2020/051261 filed Sep. 17, 2020, bioproduction in modified host cells enables production of bespoke cannabinoids or cannabinoid derivatives from simple precursors such as sugars and carboxylic acids and synthesis of cannabinoids or cannabinoid derivatives with defined stereochemistries, which is challenging to do using chemical synthesis. For example, to produce cannabinoids or cannabinoid derivatives, the modified host cells of WO2018/200888 filed Apr. 27, 2018 and WO 2020/069214 filed Sep. 26, 2019, and PCT/US2020/051261 filed Sep. 17, 2020 comprising one or more nucleic acids (e.g., heterologous nucleic acids) encoding one or more polypeptides having at least one activity of a polypeptide present in the cannabinoid or cannabinoid precursor (e.g., geranylpyrophosphate (GPP), prenyl phosphates, olivetolic acid, or hexanoyl-CoA) biosynthetic pathway can be used. In some embodiments, the nucleotide sequences encoding the one or more polypeptides involved in cannabinoid or cannabinoid precursor biosynthesis are codon-optimized.

The modified host cell may be a modified yeast cell. Yeast cells may be reasonable host cells for protein expression as well as for cannabinoid and cannabinoid derivative production because they are cultured using known conditions, grow rapidly, and are generally regarded as safe. Moreover, yeast cells contain the secretory pathway common to all eukaryotes. Manipulation of that secretory pathway in yeast host cells modified with one or more nucleic acids (e.g., heterologous nucleic acids) comprising a nucleotide sequence encoding a cannabinoid synthase polypeptide, such as the secretory pathway manipulation detailed in WO 2020/069214 filed Sep. 26, 2019, and PCT/US2020/051261 filed Sep. 17, 2020, may improve expression, folding, and enzymatic activity of the cannabinoid synthase polypeptide as well as viability of the modified yeast host cell, such as modified Saccharomyces cerevisiae. Further, as detailed in WO2018/200888 filed Apr. 27, 2018 and WO 2020/069214 filed Sep. 26, 2019, and PCT/US2020/051261 filed Sep. 17, 2020, addition of a signal sequence polypeptide to the cannabinoid synthase polypeptides, such as a secretory signal sequence polypeptide, and/or use of codon-optimized nucleotide sequences encoding cannabinoid synthase polypeptides, may improve expression and activity of the cannabinoid synthase polypeptide and viability of modified yeast host cells, such as modified Saccharomyces cerevisiae.

The modified host cells used herein, such as those detailed in WO2018/200888 filed Apr. 27, 2018 and WO 2020/069214 filed Sep. 26, 2019, and PCT/US2020/051261 filed Sep. 17, 2020, may comprise one or more heterologous nucleic acids comprising nucleotide sequences encoding one or more polypeptides involved in cannabinoid or cannabinoid precursor (e.g., geranylpyrophosphate (GPP), prenyl phosphates, olivetolic acid, or hexanoyl-CoA) biosynthesis. Such polypeptides may include, but are not limited to: a geranyl pyrophosphate:olivetolic acid geranyltransferase (GOT) polypeptide, a tetraketide synthase (TKS) polypeptide, an olivetolic acid cyclase (OAC) polypeptide, one or more polypeptides having at least one activity of a polypeptide present in the mevalonate (MEV) pathway (e.g., one or more MEV pathway polypeptides), an acyl-activating enzyme (AAE) polypeptide, a polypeptide that generates GPP (e.g., a geranyl pyrophosphate synthetase (GPPS) polypeptide), a polypeptide that condenses two molecules of acetyl-CoA to generate acetoacetyl-CoA (e.g., an acetoacetyl-CoA thiolase polypeptide), a pyruvate decarboxylase polypeptide, and a cannabinoid synthase polypeptide. In some embodiments, the nucleotide sequences encoding the one or more polypeptides involved in cannabinoid or cannabinoid precursor biosynthesis are codon-optimized.

As detailed in WO2018/200888 filed Apr. 27, 2018 and WO 2020/069214 filed Sep. 26, 2019, and PCT/US2020/051261 filed Sep. 17, 2020, the polypeptides involved in cannabinoid or cannabinoid precursor biosynthesis and the nucleotide sequences encoding the polypeptides involved in cannabinoid or cannabinoid precursor biosynthesis may be derived from any suitable source, for example, bacteria, yeast, fungi, algae, human, plant (e.g., Cannabis), or mouse. In some embodiments, the disclosure also encompasses orthologous genes encoding the polypeptides involved in cannabinoid or cannabinoid precursor biosynthesis disclosed herein. Exemplary polypeptides involved in cannabinoid or cannabinoid precursor biosynthesis disclosed herein may also include a full-length polypeptide involved in cannabinoid or cannabinoid precursor biosynthesis, a fragment of a polypeptide involved in cannabinoid or cannabinoid precursor biosynthesis, a variant of a polypeptide involved in cannabinoid or cannabinoid precursor biosynthesis, a truncated polypeptide involved in cannabinoid or cannabinoid precursor biosynthesis, or a fusion polypeptide that has at least one activity of a polypeptide involved in cannabinoid or cannabinoid precursor biosynthesis. The disclosure also provides for nucleotide sequences encoding polypeptides involved in cannabinoid or cannabinoid precursor biosynthesis, such as, a full-length polypeptide involved in cannabinoid or cannabinoid precursor biosynthesis, a fragment of a polypeptide involved in cannabinoid or cannabinoid precursor biosynthesis, a variant of a polypeptide involved in cannabinoid or cannabinoid precursor biosynthesis, a truncated polypeptide involved in cannabinoid or cannabinoid precursor biosynthesis, or a fusion polypeptide that has at least one activity of a polypeptide involved in cannabinoid or cannabinoid precursor biosynthesis. In some embodiments, the nucleotide sequences encoding the polypeptides involved in cannabinoid or cannabinoid precursor biosynthesis are codon-optimized.

As detailed in WO 2020/069214 filed Sep. 26, 2019, and PCT/US2020/051261 filed Sep. 17, 2020, secretory pathway polypeptides with modulated expression in the modified host cells used herein may include, but are not limited to: chaperone or co-chaperone polypeptides, flavin adenine dinucleotide (FAD) synthetase polypeptides, flavin mononucleotide (FMN) synthetase polypeptides, glycosidase polypeptides, glycosyltransferase polypeptides, peptidyl-prolyl isomerase polypeptides, protein disulfide isomerase polypeptides, thiol oxidase polypeptides, fatty acid desaturase polypeptides, protein transport and trafficking polypeptides, signal peptidase or signal peptidase complex polypeptides, polypeptides involved in unfolded protein response (UPR), polypeptides involved in endoplasmic reticulum-associated degradation (ERAD), polypeptides involved in protein translocation into the endoplasmic reticulum, polypeptides involved in cell wall assembly, polypeptides involved in vacuolar protein sorting (including vacuolar proteinase polypeptides), polypeptides involved in lipid droplet assembly, and polypeptides involved in regulation of lipid metabolism. Expression of secretory pathway polypeptides may be modulated by introducing into a host cell one or more heterologous nucleic acids comprising nucleotide sequences encoding one or more secretory pathway polypeptides and/or deletion or downregulation of one or more genes encoding one or more secretory pathway polypeptides in a host cell, for example as detailed in WO 2020/069214 filed Sep. 26, 2019, and PCT/US2020/051261 filed Sep. 17, 2020. In some embodiments, the nucleotide sequences encoding the one or more secretory pathway polypeptides are codon-optimized.

As detailed in WO 2020/069214 filed Sep. 26, 2019, and PCT/US2020/051261 filed Sep. 17, 2020, the secretory pathway polypeptides and the nucleotide sequences encoding the secretory pathway polypeptides may be derived from any suitable source, for example, bacteria, yeast, fungi, algae, human, plant, or mouse. In some embodiments, the secretory pathway polypeptides and the nucleotide sequences encoding the pathway polypeptides may be derived from Pichia pastoris (now known as Komagataella phaffii), Pichia finlandica, Pichia trehalophila, Pichia koclamae, Pichia membranaefaciens, Pichia opuntiae, Pichia thermotolerans, Pichia salictaria, Pichia guercuum, Pichia pijperi, Pichia stiptis, Pichia methanolica, Pichia sp., Saccharomyces cerevisiae, Saccharomyces sp., Hansenula polymorpha (now known as Pichia angusta), Yarrowia hpolytica, Kluyveromyces sp., Kluyveromyces lactis, Kluyveromyces marxianus, Schizosaccharomyces pombe, Scheffersomyces stipites, Dekkera bruxellensis, Blastobotrys adeninivorans (formerly Arxula adeninivorans), Candida albicans, Aspergillus nidulans, Aspergillus niger, Aspergillus oryzae, Trichoderma reesei, Chrysosporium lucknowense, Fusarium sp., Fusarium gramineum, Fusarium venenatum, Neurospora crassa, and the like. In some embodiments, the disclosure also encompasses orthologous genes encoding the secretory pathway polypeptides disclosed herein. Exemplary secretory pathway polypeptides disclosed herein may also include a full-length secretory pathway polypeptide, a fragment of a secretory pathway polypeptide, a variant of a secretory pathway polypeptide, a truncated secretory pathway polypeptide, or a fusion polypeptide that has at least one activity of a secretory pathway polypeptide. The disclosure also provides for nucleotide sequences encoding secretory pathway polypeptides, such as, a full-length secretory pathway polypeptide, a fragment of a secretory pathway polypeptide, a variant of a secretory pathway polypeptide, a truncated secretory pathway polypeptide, or a fusion polypeptide that has at least one activity of a secretory pathway polypeptide. In some embodiments, the nucleotide sequences encoding the secretory pathway polypeptides are codon-optimized.

### Methods of Using Host Cells to Generate Cannabinoids or Cannabinoid Derivatives

The disclosure provides methods of producing a cannabinoid or a cannabinoid derivative, such as those described herein, the method comprising culturing a modified host cell, such as those disclosed in WO2018/200888 filed Apr. 27, 2018 and WO 2020/069214 filed Sep. 26, 2019, and PCT/US2020/051261 filed Sep. 17, 2020, in a culture medium. In certain such embodiments, the produced is then purified by the methods disclosed herein to afford a preparation.

In some embodiments, culturing of the modified host cells, such as those disclosed in WO2018/200888 filed Apr. 27, 2018 and WO 2020/069214 filed Sep. 26, 2019, and PCT/US2020/051261 filed Sep. 17, 2020, in a culture medium provides for synthesis of a cannabinoid or a cannabinoid derivative, such as those described herein, in an increased amount compared to an unmodified host cell cultured under similar conditions.

The disclosure provides methods of producing a cannabinoid or a cannabinoid derivative, such as those described herein, the method comprising: culturing a modified host cell, such as those disclosed in WO2018/200888 filed Apr. 27, 2018 and WO 2020/069214 filed Sep. 26, 2019, and PCT/US2020/051261 filed Sep. 17, 2020, in a culture medium comprising a carboxylic acid. In certain such embodiments, the produced cannabinoid or cannabinoid derivative is then purified by the methods disclosed herein to afford a cannabinoid or cannabinoid derivative preparation.

In some embodiments, the modified host cell, such as those disclosed in WO2018/200888 filed Apr. 27, 2018 and WO 2020/069214 filed Sep. 26, 2019, and PCT/US2020/051261 filed Sep. 17, 2020, is cultured in a culture medium comprising a carboxylic acid. In some embodiments, the carboxylic acid may be substituted with or comprise one or more functional and/or reactive groups. Functional groups may include, but are not limited to, azido, halo (e.g., chloride, bromide, iodide, fluorine), methyl, alkyl, alkynyl, alkenyl, methoxy, alkoxy, acetyl, amino, carboxyl, carbonyl, oxo, ester, hydroxyl, thio (e.g., thiol), cyano, aryl, heteroaryl, cycloalkyl, cycloalkenyl, cycloalkylalkenyl, cycloalkylalkynyl, cycloalkenylalkyl, cycloalkenylalkenyl, cycloalkenylalkynyl, heterocyclylalkenyl, heterocyclylalkynyl, heteroarylalkenyl, heteroarylalkynyl, arylalkenyl, arylalkynyl, spirocyclyl, heterospirocyclyl, heterocyclyl, thioalkyl (or alkylthio), arylthio, heteroarylthio, sulfone, sulfonyl, sulfoxide, amido, alkylamino, dialkylamino, arylamino, alkylarylamino, diarylamino, N-oxide, imide, enamine, imine, oxime, hydrazone, nitrile, aralkyl, cycloalkylalkyl, haloalkyl, heterocyclylalkyl, heteroarylalkyl, nitro, thioxo, and the like. Reactive groups may include, but are not necessarily limited to, azide, halogen, carboxyl, carbonyl, amine (e.g., alkyl amine (e.g., lower alkyl amine), aryl amine), ester (e.g., alkyl ester (e.g., lower alkyl ester, benzyl ester), aryl ester, substituted aryl ester), cyano, thioester, thioether, sulfonyl halide, alcohol, thiol, succinimidyl ester, isothiocyanate, iodoacetamide, maleimide, hydrazine, alkynyl, alkenyl, and the like. In some embodiments, the reactive group is selected from a carboxyl, a carbonyl, an amine, an ester, thioester, thioether, a sulfonyl halide, an alcohol, a thiol, a succinimidyl ester, an isothiocyanate, an iodoacetamide, a maleimide, an azide, an alkyne, an alkene, and a hydrazine. Functional and reactive groups may be unsubstituted or substituted with one or more functional or reactive groups.

In some embodiments, the carboxylic acid is isotopically- or radio-labeled. In some embodiments, the carboxylic acid may be an enantiomer or disastereomer. In some embodiments, the carboxylic acid may be the (S)-enantiomer. In some embodiments, the carboxylic acid may be the (R)-enantiomer. In some embodiments, the carboxylic acid may be the (+) or (−) enantiomer. In some embodiments, the carboxylic acid may include a double bond or a fused ring. In certain such embodiments, the double bond or fused ring may be cis or trans, unless the configuration is specifically defined. If the carboxylic acid contains a double bond, the substituent may be in the E or Z configuration, unless the configuration is specifically defined.

In some embodiments, the carboxylic acid comprises a C═C group. In some embodiments, the carboxylic acid comprises an alkyne group. In some embodiments, the carboxylic acid comprises an N₃ group. In some embodiments, the carboxylic acid comprises a halogen. In some embodiments, the carboxylic acid comprises a CN group. In some embodiments, the carboxylic acid comprises iodo. In some embodiments, the carboxylic acid comprises bromo. In some embodiments, the carboxylic acid comprises chloro. In some embodiments, the carboxylic acid comprises fluoro. In some embodiments, the carboxylic acid comprises a carbonyl. In some embodiments, the carboxylic acid comprises an acetyl group. In some embodiments, the carboxylic acid comprises an alkyl group. In some embodiments, the carboxylic acid comprises an aryl group.

Carboxylic acids may include, but are not limited to, unsubstituted or substituted C₃-C₁₈ fatty acids, C₃-C₁₈ carboxylic acids, C₁-C₁₈ carboxylic acids, butyric acid, isobutyric acid, valeric acid, hexanoic acid, heptanoic acid, octanoic acid, nonanoic acid, decanoic acid, undecanoic acid, lauric acid, myristic acid, C₁₅-C₁₈ fatty acids, C₁₅-C₁₈ carboxylic acids, fumaric acid, itaconic acid, malic acid, succinic acid, maleic acid, malonic acid, glutaric acid, glucaric acid, oxalic acid, adipic acid, pimelic acid, suberic acid, azelaic acid, sebacic acid, dodecanedioic acid, glutaconic acid, ortho-phthalic acid, isophthalic acid, terephthalic acid, citric acid, isocitric acid, aconitic acid, tricarballylic acid, and trimesic acid. Carboxylic acids may include unsubstituted or substituted C₁-C₁₈ carboxylic acids. Carboxylic acids may include unsubstituted or substituted C₃-C₁₈ carboxylic acids. Carboxylic acids may include unsubstituted or substituted C₃-C₁₂ carboxylic acids. Carboxylic acids may include unsubstituted or substituted C₄-C₁₀ carboxylic acids. In some embodiments, the carboxylic acid is an unsubstituted or substituted C₄ carboxylic acid. In some embodiments, the carboxylic acid is an unsubstituted or substituted C₅ carboxylic acid. In some embodiments, the carboxylic acid is an unsubstituted or substituted C₆ carboxylic acid. In some embodiments, the carboxylic acid is an unsubstituted or substituted C₇ carboxylic acid. In some embodiments, the carboxylic acid is an unsubstituted or substituted C₅ carboxylic acid. In some embodiments, the carboxylic acid is an unsubstituted or substituted C₉ carboxylic acid. In some embodiments, the carboxylic acid is an unsubstituted or substituted C₁₀ carboxylic acid. In some embodiments, the carboxylic acid is unsubstituted or substituted butyric acid. In some embodiments, carboxylic acid is unsubstituted or substituted valeric acid. In some embodiments, the carboxylic acid is unsubstituted or substituted hexanoic acid. In some embodiments, the carboxylic acid is unsubstituted or substituted heptanoic acid. In some embodiments, the carboxylic acid is unsubstituted or substituted octanoic acid. In some embodiments, the carboxylic acid is unsubstituted or substituted nonanoic acid. In some embodiments, the carboxylic acid is unsubstituted or substituted decanoic acid.

Carboxylic acids may include, but are not limited to, 2-methylhexanoic acid, 3-methylhexanoic acid, 4-methylhexanoic acid, 5-methylhexanoic acid, 2-hexenoic acid, 3-hexenoic acid, 4-hexenoic acid, 5-hexenoic acid, 5-chlorovaleric acid, 5-aminovaleric acid, 5-cyanovaleric acid, 5-(methylsulfanyl)valeric acid, 5-hydroxyvaleric acid, 5-phenylvaleric acid, 2,3-dimethylhexanoic acid, d3-hexanoic acid, 4-pentynoic acid, trans-2-pentenoic acid, 5-hexynoic acid, trans-2-hexenoic acid, 6-heptynoic acid, trans-2-octenoic acid, trans-2-nonenoic acid, 4-phenylbutyric acid, 6-phenylhexanoic acid, 7-phenylheptanoic acid, and the like. In some embodiments, the carboxylic acid is 2-methylhexanoic acid. In some embodiments, the carboxylic acid is 3-methylhexanoic acid. In some embodiments, the carboxylic acid is 4-methylhexanoic acid. In some embodiments, the carboxylic acid is 5-methylhexanoic acid. In some embodiments, the carboxylic acid is 2-hexenoic acid. In some embodiments, the carboxylic acid is 3-hexenoic acid. In some embodiments, the carboxylic acid is 4-hexenoic acid. In some embodiments, the carboxylic acid is 5-hexenoic acid. In some embodiments, the carboxylic acid is 5-chlorovaleric acid. In some embodiments, the carboxylic acid is 5-aminovaleric acid. In some embodiments, the carboxylic acid is 5-cyanovaleric acid. In some embodiments, the carboxylic acid is 5-(methylsulfanyl)valeric acid. In some embodiments, the carboxylic acid is 5-hydroxyvaleric acid. In some embodiments, the carboxylic acid is 5-phenylvaleric acid. In some embodiments, the carboxylic acid is 2,3-dimethylhexanoic acid. In some embodiments, the carboxylic acid is d3-hexanoic acid. In some embodiments, the carboxylic acid is 4-pentynoic acid. In some embodiments, the carboxylic acid is trans-2-pentenoic acid. In some embodiments, the carboxylic acid is 5-hexynoic acid. In some embodiments, the carboxylic acid is trans-2-hexenoic acid. In some embodiments, the carboxylic acid is 6-heptynoic acid. In some embodiments, the carboxylic acid is trans-2-octenoic acid. In some embodiments, the carboxylic acid is trans-2-nonenoic acid. In some embodiments, the carboxylic acid is 4-phenylbutyric acid. In some embodiments, the carboxylic acid is 6-phenylhexanoic acid. In some embodiments, the carboxylic acid is 7-phenylheptanoic acid.

In some embodiments wherein the modified host cell, such as those disclosed in WO2018/200888 filed Apr. 27, 2018 and WO 2020/069214 filed Sep. 26, 2019, and PCT/US2020/051261 filed Sep. 17, 2020, is cultured in a culture medium comprising a carboxylic acid, the carboxylic acid is an unsubstituted or substituted C₃-C₁₈ carboxylic acid. In certain such embodiments, the unsubstituted or substituted C₃-C₁₈ carboxylic acid is an unsubstituted or substituted hexanoic acid. In some embodiments, the cannabinoid or cannabinoid derivative is produced in an amount of more than 100 mg/L culture medium. In some embodiments, the cannabinoid or cannabinoid derivative is produced in an amount of more than 50 mg/L culture medium. In some embodiments, the cannabinoid or cannabinoid derivative is produced in an amount of more than 1 g/L culture medium. In some embodiments, the cannabinoid or cannabinoid derivative is produced in an amount of more than 10 g/L culture medium. In some embodiments, the cannabinoid or cannabinoid derivative is produced in an amount of more than 20 g/L culture medium.

In some embodiments wherein the modified host cell, such as those disclosed in WO2018/200888 filed Apr. 27, 2018 and WO 2020/069214 filed Sep. 26, 2019, and PCT/US2020/051261 filed Sep. 17, 2020, is cultured in a culture medium comprising a carboxylic acid, the carboxylic acid is butyric acid, valeric acid, hexanoic acid, octanoic acid, 2-methylhexanoic acid, 3-methylhexanoic acid, 4-methylhexanoic acid, 5-methylhexanoic acid, 2-hexenoic acid, 3-hexenoic acid, 4-hexenoic acid, 5-hexenoic acid, heptanoic acid, 5-chlorovaleric acid, 5-(methylsulfanyl)valeric acid, 4-pentynoic acid, trans-2-pentenoic acid, 5-hexynoic acid, trans-2-hexenoic acid, 6-heptynoic acid, trans-2-octenoic acid, nonanoic acid, trans-2-nonenoic acid, decanoic acid, undecanoic acid, dodecanoic acid, myristic acid, 4-phenylbutyric acid, 5-phenylvaleric acid, 6-phenylhexanoic acid, 7-phenylheptanoic acid, isobutyric acid, fumaric acid, itaconic acid, malic acid, succinic acid, maleic acid, malonic acid, glutaric acid, glucaric acid, oxalic acid, adipic acid, pimelic acid, suberic acid, azelaic acid, sebacic acid, dodecandioic acid, glutaconic acid, ortho-phthalic acid, isophthalic acid, terephthalic acid, citric acid, isocitric acid, aconitic acid, tricarballylic acid, trimesic acid, 5-aminovaleric acid, 5-cyanovaleric acid, 5-hydroxyvaleric acid, or 2,3-dimethylhexanoic acid. In some embodiments, the cannabinoid or cannabinoid derivative is produced in an amount of more than 100 mg/L culture medium. In some embodiments, the cannabinoid or cannabinoid derivative is produced in an amount of more than 50 mg/L culture medium. In some embodiments, the cannabinoid or cannabinoid derivative is produced in an amount of more than 1 g/L culture medium. In some embodiments, the cannabinoid or cannabinoid derivative is produced in an amount of more than 10 g/L culture medium. In some embodiments, the cannabinoid or cannabinoid derivative is produced in an amount of more than 20 g/L culture medium.

In some embodiments wherein the modified host cell, such as those disclosed in WO2018/200888 filed Apr. 27, 2018 and WO 2020/069214 filed Sep. 26, 2019, and PCT/US2020/051261 filed Sep. 17, 2020, is cultured in a culture medium comprising a carboxylic acid, the carboxylic acid is a carboxylic acid of Formula (II):

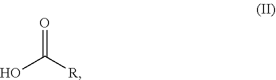

wherein R is a C₁-C₁₈alkyl group substituted with R^{a} or R is a C₂-C₁₈alkenyl group substituted with R^{a}, wherein R^{a} is —S—C₁-C₆alkyl or —S—C₂-C₆alkenyl. In some embodiments, the cannabinoid or cannabinoid derivative is produced in an amount of more than 100 mg/L culture medium. In some embodiments, the cannabinoid or cannabinoid derivative is produced in an amount of more than 50 mg/L culture medium. In some embodiments, the cannabinoid or cannabinoid derivative is produced in an amount of more than 1 g/L culture medium. In some embodiments, the cannabinoid or cannabinoid derivative is produced in an amount of more than 10 g/L culture medium. In some embodiments, the cannabinoid or cannabinoid derivative is produced in an amount of more than 20 g/L culture medium.

The disclosure also provides methods of producing a cannabinoid or a cannabinoid derivative, such as those described herein, the method comprising: culturing a modified host cell, such as those disclosed in WO2018/200888 filed Apr. 27, 2018 and WO 2020/069214 filed Sep. 26, 2019, and PCT/US2020/051261 filed Sep. 17, 2020, in a culture medium comprising olivetolic acid or an olivetolic acid derivative. In certain such embodiments, the produced cannabinoid or cannabinoid derivative is then purified by the methods disclosed herein to afford a cannabinoid or cannabinoid derivative preparation.

Olivetolic acid derivatives used herein may be substituted with or comprise one or more reactive and/or functional groups as disclosed herein. In some embodiments, an olivetolic acid derivative may lack one or more chemical moieties found in olivetolic acid. In some embodiments when the culture medium comprises an olivetolic acid derivative, the olivetolic acid derivative is orsellinic acid. In some embodiments when the culture medium comprises an olivetolic acid derivative, the olivetolic acid derivative is divarinic acid. In some embodiments, the cannabinoid or cannabinoid derivative is produced in an amount of more than 100 mg/L culture medium. In some embodiments, the cannabinoid or cannabinoid derivative is produced in an amount of more than 50 mg/L culture medium. In some embodiments, the cannabinoid or cannabinoid derivative is produced in an amount of more than 1 g/L culture medium. In some embodiments, the cannabinoid or cannabinoid derivative is produced in an amount of more than 10 g/L culture medium. In some embodiments, the cannabinoid or cannabinoid derivative is produced in an amount of more than 20 g/L culture medium.

### Exemplary Cell Culture Conditions

Suitable media for culturing modified host cells, such as those disclosed in WO2018/200888 filed Apr. 27, 2018 and WO 2020/069214 filed Sep. 26, 2019, and PCT/US2020/051261 filed Sep. 17, 2020, may include standard culture media (e.g., Luria-Bertani broth, optionally supplemented with one or more additional agents, such as an inducer (e.g., where nucleic acids disclosed herein are under the control of an inducible promoter, etc.); standard yeast culture media; and the like). In some embodiments, the culture medium can be supplemented with one or more fermentable sugars (e.g., a hexose sugar or a pentose sugar, e.g., glucose, xylose, galactose, and the like). In some embodiments, the culture medium can be supplemented with unsubstituted or substituted hexanoic acid, carboxylic acids other than unsubstituted or substituted hexanoic acid, olivetolic acid, or olivetolic acid derivatives. In some embodiments, the culture medium can be supplemented with pretreated cellulosic feedstock (e.g., wheat grass, wheat straw, barley straw, sorghum, rice grass, sugarcane straw, bagasse, switchgrass, corn stover, corn fiber, grains, or any combination thereof). In some embodiments, the culture medium can be supplemented with oleic acid. In some embodiments, the culture medium comprises a non-fermentable carbon source. In certain such embodiments, the non-fermentable carbon source comprises ethanol. In some embodiments, the suitable media comprises an inducer. In certain such embodiments, the inducer comprises galactose. In some embodiments, the inducer comprises KH₂PO₄, galactose, glucose, sucrose, maltose, an amino acid (e.g., methionine, lysine), CuSO₄, a change in temperature (e.g., 30° C. to 37° C.), a change in pH (e.g., pH 6 to pH 4), a change in oxygen level (e.g., 20% to 1% dissolved oxygen levels), addition of hydrogen peroxide or superoxide-generating drug menadione, tunicamycin, expression of proteins prone to misfolding (e.g., cannabinoid synthases), estradiol, or doxycycline. Additional induction systems are detailed herein.

The carbon source in the suitable media can vary significantly, from simple sugars like glucose to more complex hydrolysates of other biomass, such as yeast extract. The addition of salts generally provide essential elements such as magnesium, nitrogen, phosphorus, and sulfur to allow the cells to synthesize polypeptides and nucleic acids. The suitable media can also be supplemented with selective agents, such as antibiotics, to select for the maintenance of certain plasmids and the like. For example, if a microorganism is resistant to a certain antibiotic, such as ampicillin or tetracycline, then that antibiotic can be added to the medium in order to prevent cells lacking the resistance from growing. The suitable media can be supplemented with other compounds as necessary to select for desired physiological or biochemical characteristics, such as particular amino acids and the like.

In some embodiments, modified host cells, such as those disclosed in WO2018/200888 filed Apr. 27, 2018 and WO 2020/069214 filed Sep. 26, 2019, and PCT/US2020/051261 filed Sep. 17, 2020, are grown in minimal medium or minimal media. As used herein, the terms “minimal medium” or “minimal media” may refer to media comprising a defined composition of nutrients, generally chosen for minimal cost, while still allowing for robust growth and production. As used herein, the terms “minimal medium” or “minimal media” may refer to media containing: (1) one or more carbon sources for cellular (e.g., bacterial or yeast) growth; (2) various salts, which can vary among cellular (e.g., bacterial or yeast) species and growing conditions; (3) vitamins and trace elements; and (4) water. Generally, but not always, minimal media lacks one or more amino acids (e.g., 1, 2, 3, 4, 5, 6, 7, 8, 9, 10, or more amino acids). Minimal media may also comprise growth factors, inducers, and repressors.

In some embodiments, modified host cells, such as those disclosed in WO2018/200888 filed Apr. 27, 2018 and WO 2020/069214 filed Sep. 26, 2019, and PCT/US2020/051261 filed Sep. 17, 2020, are grown in defined medium or defined media (also referred to as defined minimal media or defined minimal medium). As used herein, the terms “defined medium” or “defined media” may refer to growth medium in which the exact chemical composition is known. This composition may be in excess of what is minimally required for growth.

In some embodiments, the minimal media or minimal medium or defined medium or defined media comprises galactose, glucose, ammonium sulfate, potassium phosphate, magnesium sulfate, succinate, D-biotin, Ca-D-pantothenate, nicotinic acid, myo-inositol, thiamine hydrochloride, pyridoxal hydrochloride, p-aminobenzoic acid, EDTA, zinc sulfate heptahydrate, manganese chloride dihydrate, cobalt chloride hexahydrate, copper sulfate pentahydrate, sodium molybdate dihydrate, calcium chloride dihydrate, and iron sulfate heptahydrate. In certain such embodiments, the minimal media or minimal medium or defined medium or defined media comprises 40 g/L galactose, 1 g/L glucose, 15 g/L ammonium sulfate, 8 g/L potassium phosphate, 6.15 g/L magnesium sulfate, 5.9 g/L succinate, 60 μg/L D-biotin, 120 μg/L Ca-D-pantothenate, 120 μg/L nicotinic acid, 300 μg/L myo-inositol, 60 μg/L thiamine hydrochloride, 60 μg/L pyridoxal hydrochloride, 2.5 μg/L p-aminobenzoic acid, 150 mg/L EDTA, 57.5 mg/L zinc sulfate heptahydrate, 2.6 mg/L manganese chloride dihydrate, 4.7 mg/L cobalt chloride hexahydrate, 5 mg/L copper sulfate pentahydrate, 4.8 mg/L sodium molybdate dihydrate, 29 mg/L calcium chloride dihydrate, and 28 mg/L iron sulfate heptahydrate. In some embodiments, the minimal medium or minimal media or defined medium or defined media comprises a carboxylic acid (e.g., 1 mM olivetolic acid, 1 mM olivetolic acid derivative, 2 mM unsubstituted or substituted hexanoic acid, or 2 mM of a carboxylic acid other than unsubstituted or substituted hexanoic acid). In some embodiments, minimal media or minimal medium or defined medium or defined media affords higher biomass formation in a fermentation tank compared to rich medium or rich media. Minimal media or minimal medium or defined medium or defined media may permit regulation of the components of the media or medium such that everything is available in the desired proportions during fermentation. By comparison, rich media or rich medium may permit accumulation of unconsumed components during fermentation, slowing or inhibiting cell growth as these components accumulate over the timecourse of the fermentation. Minimal media or minimal medium or defined medium or defined media affords more homogeneity and reproducibility between media batches, enhanced stability over time, eased technology transfer of media formulations, and enhanced product quality and purity compared to rich medium or rich media. In some embodiments, the modified host cell, such as those disclosed in WO2018/200888 filed Apr. 27, 2018 and WO 2020/069214 filed Sep. 26, 2019, and PCT/US2020/051261 filed Sep. 17, 2020, can produce a cannabinoid or cannabinoid derivative when cultured in a defined medium or minimal medium.

In some embodiments, modified host cells, such as those disclosed in WO2018/200888 filed Apr. 27, 2018 and WO 2020/069214 filed Sep. 26, 2019, and PCT/US2020/051261 filed Sep. 17, 2020, are grown in rich medium or rich media. The components of rich media or rich medium are often undefined, as the yeast extract peptone dextrose (YPD) is largely composed of undefined extracts that vary batch to batch. This variation can result in differences in bioprocess performance during fermentation. In some embodiments, the rich medium or rich media comprises yeast extract peptone dextrose (YPD) media comprising water, yeast extract, Bacto peptone, and dextrose (glucose). In certain such embodiments, the rich medium or rich media comprises yeast extract peptone dextrose (YPD) media comprising water, 10 g/L yeast extract, 20 g/L Bacto peptone, and 20 g/L dextrose (glucose). In some embodiments, the rich medium or rich media comprises YP+galactose and glucose. In some embodiments, the rich medium or rich media comprises YP+20 g/L galactose or YP+40 g/L galactose and 1 g/L glucose. In some embodiments, the rich medium or rich media comprises a carboxylic acid (e.g., 1 mM olivetolic acid, 1 mM olivetolic acid derivative, 2 mM unsubstituted or substituted hexanoic acid, or 2 mM of a carboxylic acid other than unsubstituted or substituted hexanoic acid). In some embodiments, rich medium or rich media affords greater cell density in fermentation compared to minimal media or minimal medium or defined media or defined medium. In some embodiments, the modified host cell, such as those disclosed in WO2018/200888 filed Apr. 27, 2018 and WO 2020/069214 filed Sep. 26, 2019, and PCT/US2020/051261 filed Sep. 17, 2020, can produce a cannabinoid or cannabinoid derivative when cultured in a rich medium.

Materials and methods suitable for the maintenance and growth of the modified host cells, such as those disclosed in WO2018/200888 filed Apr. 27, 2018 and WO 2020/069214 filed Sep. 26, 2019, and PCT/US2020/051261 filed Sep. 17, 2020, are described herein. Other materials and methods suitable for the maintenance and growth of cell (e.g., bacterial or yeast) cultures are well known in the art. Exemplary techniques can be found in International Publication No. WO2009/076676, U.S. patent application Ser. No. 12/335,071 (U.S. Publ. No. 2009/0203102), WO 2010/003007, US Publ. No. 2010/0048964, WO 2009/132220, US Publ. No. 2010/0003716, Manual of Methods for General Bacteriology Gerhardt et al, eds), American Society for Microbiology, Washington, D.C. (1994) or Brock in Biotechnology: A Textbook of Industrial Microbiology, Second Edition (1989) Sinauer Associates, Inc., Sunderland, Mass.

Standard cell culture conditions can be used to culture the modified host cells (see, for example, WO 2004/033646 and references cited therein). In some embodiments, cells are grown and maintained at an appropriate temperature, gas mixture, and pH (such as at about 20° C. to about 37° C., at about 0.04% to about 84% CO₂, at about 0% to about 100% dissolved oxygen, and at a pH between about 2.0 to about 9.0). In some embodiments, modified host cells are grown at about 34° C. in a suitable cell culture medium. In some embodiments, modified host cells are grown at about 20° C. to about 37° C. in a suitable cell culture medium. While the growth optimum for S. cerevisiae is about 30° C., culturing cells at a higher temperature, e.g., 34° C. may be advantageous by reducing the costs to cool industrial fermentation tanks. In some embodiments, modified host cells are grown at about 20° C., about 21° C., about 22° C., about 23° C., about 24° C., about 25° C., about 26° C., about 27° C., about 28° C., about 29° C., about 30° C., about 31° C., about 32° C., about 33° C., about 34° C., about 35° C., about 36° C., or about 37° C. in a suitable cell culture medium. In some embodiments, the pH ranges for fermentation are between about pH 3.0 to about pH 9.0 (such as about pH 3.0, about pH 3.5, about pH 4.0, about pH 4.5, about pH 5.0, about pH 5.5, about pH 6.0, about pH 6.5, about pH 7.0, about pH 7.5, about pH 8.0, about pH 8.5, about pH 6.0 to about pH 8.0 or about pH 6.5 to about pH 7.0). In some embodiments, the pH ranges for fermentation are between about pH 4.5 to about pH 5.5. In some embodiments, the pH ranges for fermentation are between about pH 4.0 to about pH 6.0. In some embodiments, the pH ranges for fermentation are between about pH 3.0 to about pH 6.0. In some embodiments, the pH ranges for fermentation are between about pH 3.0 to about pH 5.5. In some embodiments, the pH ranges for fermentation are between about pH 3.0 to about pH 5.0. In some embodiments, the dissolved oxygen is between about 0% to about 10%, about 0% to about 20%, about 0% to about 30%, about 0% to about 40%, about 0% to about 50%, about 0% to about 60%, about 0% to about 70%, about 0% to about 80%, about 0% to about 90%, about 5% to about 10%, about 5% to about 20%, about 5% to about 30%, about 5% to about 40%, about 5% to about 50%, about 5% to about 60%, about 5% to about 70%, about 5% to about 80%, about 5% to about 90%, about 10% to about 20%, about 10% to about 30%, about 10% to about 40% or about 10% to about 50%. In some embodiments, the CO₂ level is between about 0.04% to about 0.1% CO₂, about 0.04% to about 1% CO₂, about 0.04% to about 5% CO₂, about 0.04% to about 10% CO₂, about 0.04% to about 20% CO₂, about 0.04% to about 30% CO₂, about 0.04% to about 40% CO₂, about 0.04% to about 50% CO₂, about 0.04% to about 60% CO₂, about 0.04% to about 70% CO₂, about 0.1% to about 5% CO₂, about 0.1% to about 10% CO₂, about 0.1% to about 20% CO₂, about 0.1% to about 30% CO₂, about 0.1% to about 40% CO₂, about 0.1% to about 50% CO₂, about 1% to about 5% CO₂, about 1% to about 10% CO₂, about 1% to about 20% CO₂, about 1% to about 30% CO₂, about 1% to about 40% CO₂, about 1% to about 50% CO₂, about 5% to about 10% CO₂, about 10% to about 20% CO₂, about 10% to about 30% CO₂, about 10% to about 40% CO₂, about 10% to about 50% CO₂, about 10% to about 60% CO₂, about 10% to about 70% CO₂, about 10% to about 80% CO₂, about 50% to about 60% CO₂, about 50% to about 70% CO₂, or about 50% to about 80% CO₂. Modified host cells can be grown under aerobic, anoxic, microaerobic, or anaerobic conditions based on the requirements of the cells.

Standard culture conditions and modes of fermentation, such as batch, fed-batch, or continuous fermentation that can be used are described in International Publication No. WO 2009/076676, U.S. patent application Ser. No. 12/335,071 (U.S. Publ. No. 2009/0203102), WO 2010/003007, US Publ. No. 2010/0048964, WO 2009/132220, US Publ. No. 2010/0003716, the contents of each of which are incorporated by reference herein in their entireties. Batch and Fed-Batch fermentations are common and well known in the art and examples can be found in Brock, Biotechnology: A Textbook of Industrial Microbiology, Second Edition (1989) Sinauer Associates, Inc.

### Production of Cannabinoids or Cannabinoid Derivatives

The present disclosure provides for production of a cannabinoid or a cannabinoid derivative, which can then purified by the methods disclosed herein to afford a cannabinoid or cannabinoid derivative preparation. In some embodiments, a method of the present disclosure provides for production of a cannabinoid or a cannabinoid derivative, such as those disclosed herein, by modified host cells, such as those disclosed in WO2018/200888 filed Apr. 27, 2018 and WO 2020/069214 filed Sep. 26, 2019, and PCT/US2020/051261 filed Sep. 17, 2020. In some embodiments, a method of the present disclosure provides for production of a cannabinoid or a cannabinoid derivative, such as those disclosed herein, by modified host cells in an amount of from about 1 mg/L culture medium to about 30 g/L culture medium. In some embodiments, a method of the present disclosure provides for production of a cannabinoid or a cannabinoid derivative in an amount of from about 1 mg/L culture medium to about 20 g/L culture medium. In some embodiments, a method of the present disclosure provides for production of a cannabinoid or a cannabinoid derivative in an amount of from about 1 mg/L culture medium to about 10 g/L culture medium. In some embodiments, a method of the present disclosure provides for production of a cannabinoid or a cannabinoid derivative in an amount of from about 1 mg/L culture medium to about 1 g/L culture medium. In some embodiments, a method of the present disclosure provides for production of a cannabinoid or a cannabinoid derivative in an amount of from about 1 mg/L culture medium to about 100 mg/L culture medium. For example, in some embodiments, a method of the present disclosure provides for production of a cannabinoid or a cannabinoid derivative in an amount of from about 1 mg/L culture medium to about 5 mg/L culture medium, from about 5 mg/L culture medium to about 10 mg/L culture medium, from about 10 mg/L culture medium to about 25 mg/L culture medium, from about 25 mg/L culture medium to about 50 mg/L culture medium, from about 50 mg/L culture medium to about 75 mg/L culture medium, or from about 75 mg/L culture medium to about 100 mg/L culture medium, or from about 100 mg/L culture medium to about lg/L culture medium, or from about 1 g/L culture medium to about 5 g/L culture medium, or from about 5 g/L culture medium to about 10 g/L culture medium, or from about 10 g/L culture medium to about 20 g/L culture medium. In some embodiments, a method of the present disclosure provides for production of a cannabinoid or a cannabinoid derivative in an amount of from about 100 mg/L culture medium to about 150 mg/L culture medium, from about 150 mg/L culture medium to about 200 mg/L culture medium, from about 200 mg/L culture medium to about 250 mg/L culture medium, from about 250 mg/L culture medium to about 500 mg/L culture medium, from about 500 mg/L culture medium to about 750 mg/L culture medium, or from about 750 mg/L culture medium to about 1 g/L culture medium, or from about 1 g/L culture medium to about 5 g/L culture medium, or from about 5 g/L culture medium to about 10 g/L culture medium, or from about 10 g/L culture medium to about 20 g/L culture medium. In some embodiments, a method of the present disclosure provides for production of a cannabinoid or a cannabinoid derivative in an amount of from about from about 50 mg/L culture medium to about 100 mg/L culture medium, 50 mg/L culture medium to about 150 mg/L culture medium, from about 50 mg/L culture medium to about 200 mg/L culture medium, from about 50 mg/L culture medium to about 250 mg/L culture medium, from about 50 mg/L culture medium to about 500 mg/L culture medium, or from about 50 mg/L culture medium to about 750 mg/L culture medium.

In some embodiments, a method of the present disclosure provides for production of a cannabinoid or a cannabinoid derivative, such as those disclosed herein, in an amount of from about 50 mg/L culture medium to about 100 g/L culture medium, or more than 100 g/L culture medium. In some embodiments, a method of the present disclosure provides for production of a cannabinoid or a cannabinoid derivative, such as those disclosed herein, in an amount of from about 50 mg/L culture medium to about 500 g/L culture medium, or more than 500 g/L culture medium. In some embodiments, a method of the present disclosure provides for production of a cannabinoid or a cannabinoid derivative, such as those disclosed herein, in an amount of from about 50 mg/L culture medium to about 100 mg/L culture medium, or more than 100 mg/L culture medium. In some embodiments, a method of the present disclosure provides for production of a cannabinoid or a cannabinoid derivative, such as those disclosed herein, in an amount of more than 50 mg/L culture medium. In some embodiments, a method of the present disclosure provides for production of a cannabinoid or a cannabinoid derivative, such as those disclosed herein, in an amount of more than 100 mg/L culture medium. In some embodiments, a method of the present disclosure provides for production of a cannabinoid or a cannabinoid derivative in an amount of from about 100 mg/L culture medium to about 500 mg/L culture medium, or more than 500 mg/L culture medium. In some embodiments, a method of the present disclosure provides for production of a cannabinoid or a cannabinoid derivative in an amount of from about 500 mg/L culture medium to about 1 g/L culture medium, or more than 1 g/L culture medium. In some embodiments, a method of the present disclosure provides for production of a cannabinoid or a cannabinoid derivative in an amount of from about 1 g/L culture medium to about 10 g/L culture medium, or more than 10 g/L culture medium. In some embodiments, a method of the present disclosure provides for production of a cannabinoid or a cannabinoid derivative in an amount of from about 10 g/L culture medium to about 100 g/L culture medium, or more than 100 g/L culture medium. In some embodiments, a method of the present disclosure provides for production of a cannabinoid or a cannabinoid derivative in an amount of from about 1 g/L culture medium to about 20 g/L culture medium, or more than 20 g/L culture medium. In some embodiments, a method of the present disclosure provides for production of a cannabinoid or a cannabinoid derivative in an amount of from about 1 g/L culture medium to about 30 g/L culture medium, or more than 30 g/L culture medium. In some embodiments, a method of the present disclosure provides for production of a cannabinoid or a cannabinoid derivative in an amount of from about 1 g/L culture medium to about 40 g/L culture medium, or more than 40 g/L culture medium. In some embodiments, a method of the present disclosure provides for production of a cannabinoid or a cannabinoid derivative in an amount of from about 1 g/L culture medium to about 50 g/L culture medium, or more than 50 g/L culture medium. In some embodiments, a method of the present disclosure provides for production of a cannabinoid or a cannabinoid derivative in an amount of from about 1 g/L culture medium to about 60 g/L culture medium, or more than 60 g/L culture medium. In some embodiments, a method of the present disclosure provides for production of a cannabinoid or a cannabinoid derivative in an amount of from about 1 g/L culture medium to about 70 g/L culture medium, or more than 70 g/L culture medium. In some embodiments, a method of the present disclosure provides for production of a cannabinoid or a cannabinoid derivative in an amount of from about 1 g/L culture medium to about 80 g/L culture medium, or more than 80 g/L culture medium. In some embodiments, a method of the present disclosure provides for production of a cannabinoid or a cannabinoid derivative in an amount of from about 1 g/L culture medium to about 90 g/L culture medium, or more than 90 g/L culture medium. In some embodiments, a method of the present disclosure provides for production of a cannabinoid or a cannabinoid derivative in an amount of from about 10 g/L culture medium to about 20 g/L culture medium, or more than 20 g/L culture medium. In some embodiments, a method of the present disclosure provides for production of a cannabinoid or a cannabinoid derivative in an amount of from about 10 g/L culture medium to about 30 g/L culture medium, or more than 30 g/L culture medium. In some embodiments, a method of the present disclosure provides for production of a cannabinoid or a cannabinoid derivative in an amount of from about 10 g/L culture medium to about 40 g/L culture medium, or more than 40 g/L culture medium. In some embodiments, a method of the present disclosure provides for production of a cannabinoid or a cannabinoid derivative in an amount of from about 10 g/L culture medium to about 50 g/L culture medium, or more than 50 g/L culture medium. In some embodiments, a method of the present disclosure provides for production of a cannabinoid or a cannabinoid derivative in an amount of from about 10 g/L culture medium to about 60 g/L culture medium, or more than 60 g/L culture medium. In some embodiments, a method of the present disclosure provides for production of a cannabinoid or a cannabinoid derivative in an amount of from about 10 g/L culture medium to about 70 g/L culture medium, or more than 70 g/L culture medium. In some embodiments, a method of the present disclosure provides for production of a cannabinoid or a cannabinoid derivative in an amount of from about 10 g/L culture medium to about 80 g/L culture medium, or more than 80 g/L culture medium. In some embodiments, a method of the present disclosure provides for production of a cannabinoid or a cannabinoid derivative in an amount of from about 10 g/L culture medium to about 90 g/L culture medium, or more than 90 g/L culture medium. In some embodiments, a method of the present disclosure provides for production of a cannabinoid or a cannabinoid derivative in an amount of from about 50 g/L culture medium to about 100 g/L culture medium, or more than 100 g/L culture medium. In some embodiments, a method of the present disclosure provides for production of a cannabinoid or a cannabinoid derivative in an amount of from about 50 g/L culture medium to about 60 g/L culture medium, or more than 60 g/L culture medium. In some embodiments, a method of the present disclosure provides for production of a cannabinoid or a cannabinoid derivative in an amount of from about 50 g/L culture medium to about 70 g/L culture medium, or more than 70 g/L culture medium. In some embodiments, a method of the present disclosure provides for production of a cannabinoid or a cannabinoid derivative in an amount of from about 50 g/L culture medium to about 80 g/L culture medium, or more than 80 g/L culture medium. In some embodiments, a method of the present disclosure provides for production of a cannabinoid or a cannabinoid derivative in an amount of from about 50 g/L culture medium to about 90 g/L culture medium, or more than 90 g/L culture medium. In some embodiments, a method of the present disclosure provides for production of a cannabinoid or a cannabinoid derivative in an amount of from about 20 g/L culture medium to about 100 g/L culture medium, or more than 100 g/L culture medium. In some embodiments, a method of the present disclosure provides for production of a cannabinoid or a cannabinoid derivative in an amount of from about 20 g/L culture medium to about 30 g/L culture medium, or more than 30 g/L culture medium. In some embodiments, a method of the present disclosure provides for production of a cannabinoid or a cannabinoid derivative in an amount of from about 20 g/L culture medium to about 40 g/L culture medium, or more than 40 g/L culture medium. In some embodiments, a method of the present disclosure provides for production of a cannabinoid or a cannabinoid derivative in an amount of from about 20 g/L culture medium to about 50 g/L culture medium, or more than 50 g/L culture medium. In some embodiments, a method of the present disclosure provides for production of a cannabinoid or a cannabinoid derivative in an amount of from about 20 g/L culture medium to about 60 g/L culture medium, or more than 60 g/L culture medium. In some embodiments, a method of the present disclosure provides for production of a cannabinoid or a cannabinoid derivative in an amount of from about 20 g/L culture medium to about 70 g/L culture medium, or more than 70 g/L culture medium. In some embodiments, a method of the present disclosure provides for production of a cannabinoid or a cannabinoid derivative in an amount of from about 20 g/L culture medium to about 80 g/L culture medium, or more than 80 g/L culture medium. In some embodiments, a method of the present disclosure provides for production of a cannabinoid or a cannabinoid derivative in an amount of from about 20 g/L culture medium to about 90 g/L culture medium, or more than 90 g/L culture medium. In some embodiments, a method of the present disclosure provides for production of a cannabinoid or a cannabinoid derivative in an amount of from about 20 g/L culture medium to about 500 g/L culture medium, or more than 500 g/L culture medium.

In some embodiments, a method of the present disclosure provides for increased production of a cannabinoid or a cannabinoid derivative, such as those disclosed herein. In certain such embodiments, culturing of the modified host cell in a culture medium provides for synthesis of a cannabinoid or a cannabinoid derivative in an increased amount compared to an unmodified host cell cultured under similar conditions. The production of a cannabinoid or a cannabinoid derivative by the modified host cells may be increased by about 5% to about 1,000,000 folds compared to an unmodified host cell cultured under similar conditions. The production of a cannabinoid or a cannabinoid derivative by the modified host cells may be increased by about 10% to about 1,000,000 folds (e.g., about 50% to about 1,000,000 folds, about 1 to about 500,000 folds, about 1 to about 50,000 folds, about 1 to about 5,000 folds, about 1 to about 1,000 folds, about 1 to about 500 folds, about 1 to about 100 folds, about 1 to about 50 folds, about 5 to about 100,000 folds, about 5 to about 10,000 folds, about 5 to about 1,000 folds, about 5 to about 500 folds, about 5 to about 100 folds, about 10 to about 50,000 folds, about 50 to about 10,000 folds, about 100 to about 5,000 folds, about 200 to about 1,000 folds, about 50 to about 500 folds, or about 50 to about 200 folds) compared to the production of a cannabinoid or a cannabinoid derivative by unmodified host cells cultured under similar conditions. The production of a cannabinoid or a cannabinoid derivative by modified host cells may also be increased by at least about any of 10%, 20%, 30%, 40%, 50%, 60%, 70%, 80%, 90%, 100%, 1 fold, 2 folds, 5 folds, 10 folds, 20 folds, 50 folds, 100 folds, 200 folds, 500 folds, 1000 folds, 2000 folds, 5000 folds, 10,000 folds, 20,000 folds, 50,000 folds, 100,000 folds, 200,000 folds, 500,000 folds, or 1,000,000 folds or more compared to the production of a cannabinoid or a cannabinoid derivative by unmodified host cells cultured under similar conditions.

In some embodiments, the production of a cannabinoid or a cannabinoid derivative, such as those disclosed herein, by modified host cells may also be increased by at least about any of 10%, 20%, 30%, 40%, 50%, 60%, 70%, 80%, 90%, or 100% compared to the production of a cannabinoid or a cannabinoid derivative by unmodified host cells cultured under similar conditions.

In some embodiments, the production of a cannabinoid or a cannabinoid derivative by modified host cells may also be increased by at least about any of 1-20%, 2-20%, 5-20%, 10-20%, 15-20%, 1-15%, 1-10%, 2-15%, 2-10%, 5-15%, 10-15%, 1-50%, 10-50%, 20-50%, 30-50%, 40-50%, 50-100%, 50-60%, 50-70%, 50-80%, 50-90%, or 50-100% compared to the production of a cannabinoid or a cannabinoid derivative by unmodified host cells cultured under similar conditions.

In some embodiments, production of a cannabinoid or a cannabinoid derivative by modified host cells is determined by LC-MS analysis. In certain such embodiments, each cannabinoid or cannabinoid derivative is identified by retention time, determined from an authentic standard, and multiple reaction monitoring (MRM) transition.

In some embodiments, the modified host cell is yeast cell. In certain such embodiments, the modified host cell is cultured in a bioreactor. In some embodiments, the modified host cell is cultured in a culture medium supplemented with unsubstituted or substituted hexanoic acid, a carboxylic acid other than unsubstituted or substituted hexanoic acid, olivetolic acid, or an olivetolic acid derivative. In some embodiments, the modified yeast cell is a modified S. cerevisiae.

In some embodiments, the cannabinoid or cannabinoid derivative produced using the modified host cells or in the methods or preparations of the disclosure is in the form of a salt. In certain such embodiments, the salt is a pharmaceutically acceptable salt. In some embodiments, the salt is an acceptable salt. In some embodiments, the cannabinoid or cannabinoid derivative recovered in the methods or preparations of the disclosure is not in the form of a salt.

### Formulations of the Disclosure

The cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), may be used on their own but will generally be administered in the form of a GRAS formulations, for example a pharmaceutical formulation, in which one or more disclosed cannabinoid or cannabinoid derivative preparations is in association with an acceptable carrier, for example a pharmaceutically acceptable carrier or a carrier generally regarded as safe (GRAS). Conventional procedures for the selection and preparation of suitable GRAS formulations are described in, for example, “Pharmaceuticals—The Science of Dosage Form Designs,” M. E. Aulton, Churchill Livingstone, 1988, which is hereby incorporated by reference in its entirety. The formulations described herein (e.g., pharmaceutical formulations and GRAS formulations) may be used in connection with the methods of treatment, uses, and medicaments described herein.

The term “carrier,” as used in this disclosure, may encompass carriers, excipients, and diluents and may mean a material, composition or vehicle, such as a liquid or solid filler, diluent, excipient, solvent or encapsulating material, involved in carrying or transporting an agent, such as one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) from one organ, or portion of the body, to another organ, or portion of the body of a subject. Carriers should be selected on the basis of compatibility and the release profile properties of the desired dosage form. Carriers may also be selected because they are GRAS. Exemplary carrier materials may include, e.g., binders, suspending agents, disintegration agents, filling agents, surfactants, solubilizers, stabilizers, lubricants, wetting agents, diluents, spray-dried dispersions, and the like. See, e.g., Hoover, John E., Remington's Pharmaceutical Sciences, Mack Publishing Co., Easton, Pa. 1975.

In some embodiments, the disclosure provides for a GRAS formulation comprising a cannabinoid or cannabinoid derivative preparation disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) and an acceptable carrier. In some embodiments, the disclosure provides for a GRAS formulation comprising a cannabinoid or cannabinoid derivative preparation disclosed herein (e.g., a preparation comprising an acidic cannabinoid, an acidic cannabinoid derivative, a neutral cannabinoid, or a neutral cannabinoid derivative, or an acceptable or pharmaceutically acceptable salt of any of the foregoing) and an acceptable carrier. In some embodiments, the disclosure provides for a GRAS formulation comprising two or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) and an acceptable carrier. In some embodiments, the disclosure provides for a GRAS formulation comprising three or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) and an acceptable carrier. In some embodiments, the disclosure provides for a GRAS formulation comprising four or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) and an acceptable carrier. In some embodiments, the disclosure provides for a GRAS formulation comprising five or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) and an acceptable carrier.

In some embodiments, the disclosure provides for a pharmaceutical formulation comprising one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) and a pharmaceutically acceptable carrier. In some embodiments, the disclosure provides for a pharmaceutical formulation comprising a cannabinoid or cannabinoid derivative preparation disclosed herein (e.g., a preparation comprising an acidic cannabinoid, an acidic cannabinoid derivative, a neutral cannabinoid, or a neutral cannabinoid derivative, or a pharmaceutically acceptable salt of any of the foregoing) and a pharmaceutically acceptable carrier. In some embodiments, the disclosure provides for a pharmaceutical formulation comprising two or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) and a pharmaceutically acceptable carrier. In some embodiments, the disclosure provides for a pharmaceutical formulation comprising three or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) and a pharmaceutically acceptable carrier.

In some embodiments, the disclosure provides for a pharmaceutical formulation comprising four or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) and a pharmaceutically acceptable carrier. In some embodiments, the disclosure provides for a pharmaceutical formulation comprising five or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) and a pharmaceutically acceptable carrier.

Depending on the mode of administration, the GRAS formulation, or pharmaceutical formulation will comprise from about 0.05 to about 99 wt % (percent by weight), more particularly from about 0.05 to about 80 wt %, still more particularly from about 0.10 to about 70 wt %, and even more particularly from about 0.10 to about 50 wt % of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), all percentages by weight being based on total formulation.

The present disclosure provides a process for the preparation of a GRAS formulation of the present disclosure which comprises mixing one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) with an acceptable carrier. GRAS formulations of the disclosure can also be prepared according to conventional mixing, granulating, or coating methods.

The present disclosure provides a process for the preparation of a pharmaceutical formulation of the present disclosure which comprises mixing one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) with a pharmaceutically acceptable carrier. Pharmaceutical formulations of the disclosure can also be prepared according to conventional mixing, granulating or coating methods.

GRAS formulations or pharmaceutical formulations of the present disclosure may comprise one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) formulated together with one or more acceptable carriers or pharmaceutically acceptable carriers. In some embodiments, the one or more cannabinoid or cannabinoid derivative preparations are present in the GRAS formulations or pharmaceutical formulations in a therapeutically effective amount. Some examples of materials that can serve as acceptable carriers or pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil, and soybean oil; glycols such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the GRAS formulation or pharmaceutical formulation, according to the judgment of the formulator.

Depending on the intended mode of administration, the disclosed GRAS formulations or pharmaceutical formulations can be in solid, semi-solid or liquid dosage form, such as, for example, injectables, tablets, suppositories, pills, softgels, time-release capsules, elixirs, tinctures, oils, extracts, creams, lotions, emulsions, syrups, powders, liquids, suspensions, or the like, sometimes in unit dosages and consistent with conventional practices. These modes may include systemic or local administration such as oral, nasal, parenteral (as by intravenous (both bolus and infusion), intramuscular, or subcutaneous injection), transdermal, vaginal, buccal, rectal or topical (as by powders, ointments, or drops) administration modes. These modes may also include intracisternally, intraperitoneally, as an oral or nasal spray, or as a liquid aerosol or dry powder formulation or pharmaceutical formulation for inhalation.

Solid dosage forms for oral administration may include capsules, softgels, tablets, pills, powders, crystals, and granules. In such solid dosage forms, one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) may be mixed with at least one inert, acceptable or pharmaceutically acceptable carriers such as a diluent, fillers or extenders, binders, humectants, disintegrating agents, solution retarding agents, wetting agents, lubricants, an emulsifier or dispersing agent, or buffering agents.

Solid GRAS formulations or solid pharmaceutical formulations of a similar type may also be employed as fillers in soft and hard-filled gelatin capsules using such excipients as lactose or milk sugar as well as high molecular weight polyethylene glycols and the like.

The one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) can also be in micro-encapsulated form with one or more excipients as noted herein. The solid dosage forms of tablets, dragees, softgels, capsules, pills, crystals, and granules can be prepared with coatings and shells such as enteric coatings, release controlling coatings and other coatings well known in the art. In such solid dosage forms, one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, softgels, tablets and pills, the dosage forms may also comprise buffering agents. They may optionally contain opacifying agents and can also be of a formulation that they release the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), only, or preferentially, in a certain part of the intestinal tract, optionally, in a delayed manner. Examples of embedding compositions that can be used may include polymeric substances and waxes.

Liquid dosage forms for oral administration may include emulsions, microemulsions, solutions, suspensions, syrups, tinctures, oils, extracts, and elixirs. In addition to the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral GRAS formulations or oral pharmaceutical formulations can also include adjuvants such as wetting agents, emulsifying and suspending agents, sweetening, flavoring, and perfuming agents.

Injectable pharmaceutical formulations, for example, sterile injectable aqueous or oleaginous suspensions comprising one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) may be formulated according to the known art using suitable dispersing or wetting agents and suspending agents. The sterile injectable pharmaceutical formulation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, 1% lidocaine, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the formulation of injectables.

The injectable pharmaceutical formulations can be sterilized, for example, by filtration through a bacterial-retaining filter, or by incorporating sterilizing agents in the form of sterile solid pharmaceutical formulations that can be dissolved or dispersed in sterile water or other sterile injectable medium prior to use.

In order to prolong the effect of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing), it may desirable to slow the absorption of the one or more cannabinoid or cannabinoid derivative pharmaceutical formulations from subcutaneous or intramuscular injection. This may be accomplished by the use of a liquid suspension of crystalline or amorphous material with poor water solubility. The rate of absorption of the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) then depends upon its rate of dissolution that, in turn, may depend upon crystal size and crystalline form. Alternatively, delayed absorption of a parenterally administered one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) may be accomplished by dissolving or suspending the one or more cannabinoid or cannabinoid derivative preparations in an oil vehicle. Injectable depot forms are made by forming microencapsule matrices of the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) in biodegradable polymers such as polylactide-polyglycolide. Depending upon the ratio of the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) to polymer and the nature of the particular polymer employed, the rate of release for the one or more cannabinoid or cannabinoid derivative preparations can be controlled. Examples of other biodegradable polymers include poly(orthoesters) and poly(anhydrides). Depot injectable pharmaceutical formulations may also be prepared by entrapping the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) in liposomes or microemulsions that are compatible with body tissues.

GRAS formulations or pharmaceutical formulations for rectal or vaginal administration may be suppositories that can be prepared by mixing the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) with suitable non-irritating carriers such as cocoa butter, polyethylene glycol or a suppository wax which are solid at ambient temperature but liquid at body temperature and therefore melt in the rectum or vaginal cavity and release the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing).

The one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) can be also formulated as a suppository that can be prepared from fatty emulsions or suspensions; using polyalkylene glycols such as propylene glycol, as the carrier.

Dosage forms for topical or transdermal administration of one or more cannabinoid or cannabinoid derivative formulations disclosed herein (e.g., formulations comprising an acidic cannabinoid, an acidic cannabinoid derivative, a neutral cannabinoid, or a neutral cannabinoid derivative, or an acceptable or pharmaceutically acceptable salt of any of the foregoing) include ointments, pastes, creams, lotions, gels, powders, solutions, sprays, inhalants, oils, or patches. The one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) are admixed under sterile conditions with an acceptable or pharmaceutically acceptable carrier and any needed preservatives or buffers as may be required. Ophthalmic GRAS formulations or ophthalmic pharmaceutical formulations, ear drops, and the like are also contemplated as being within the scope of this disclosure.

The ointments, pastes, creams, lotions, gels, solutions, inhalants, or oils may contain, in addition to one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), excipients such as animal and vegetable fats, oils, waxes, paraffins, starch, tragacanth, cellulose derivatives, polyethylene glycols, silicones, bentonites, silicic acid, talc and zinc oxide, or mixtures thereof.

One or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) may also be formulated for use as topical powders and sprays that can contain, in addition to one or more cannabinoid or cannabinoid derivative preparations, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) to the body. Such dosage forms can be made by dissolving or dispensing the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) in the proper medium. Absorption enhancers can also be used to increase the flux of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) across the skin. The rate can be controlled by either providing a rate controlling membrane or by dispersing the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) in a polymer matrix or gel.

GRAS formulations or pharmaceutical formulations of the disclosure may also be formulated for delivery as a liquid aerosol or inhalable dry powder. Liquid aerosol GRAS formulations or liquid aerosol pharmaceutical formulations may be nebulized predominantly into particle sizes that can be delivered to the terminal and respiratory bronchioles. Liquid aerosol and inhalable dry powder GRAS formulations or pharmaceutical formulations may be delivered throughout the endobronchial tree to the terminal bronchioles and eventually to the parenchymal tissue.

Aerosolized GRAS formulations or aerosolized pharmaceutical formulations of the disclosure may be delivered using an aerosol forming device, such as a jet, vibrating porous plate or ultrasonic nebulizer, selected to allow the formation of an aerosol particles having with a mass medium average diameter predominantly between 1 to 5 μm. Further, the GRAS formulations or pharmaceutical formulation may have balanced osmolarity ionic strength and chloride concentration, and the smallest aerosolizable volume able to deliver a dose of the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing). Additionally, the aerosolized GRAS formulation or aerosolized pharmaceutical formulation may not impair negatively the functionality of the airways and does not cause undesirable side effects.

Aerosolization devices suitable for administration of aerosol GRAS formulations or aerosol pharmaceutical formulations of the disclosure include, for example, jet, vibrating porous plate, ultrasonic nebulizers and energized dry powder inhalers, that are able to nebulize the GRAS formulations or pharmaceutical formulations of the disclosure into aerosol particle size predominantly in the size range from 1-5 μm. Predominantly in this application means that at least 70% but optionally more than 90% of all generated aerosol particles are 1 to 5 μm range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AeroNeb and AeroDose vibrating porous plate nebulizers (AeroGen, Inc., Sunnyvale, Calif.), Sidestream7 nebulizers (Medic-Aid Ltd., West Sussex, England), Pari LC₇ and Pari LC Star7 jet nebulizers (Pari Respiratory Equipment, Inc., Richmond, Va.), and Aerosonic (DeVilbiss Medizinische Produkte (Deutschland) GmbH, Heiden, Germany) and μLtraAire7 (Omron Healthcare, Inc., Vernon Hills, Ill.) ultrasonic nebulizers.

GRAS formulations or pharmaceutical formulations of the disclosure may also be formulated into a vaporizable formulation. In certain such embodiments, the GRAS formulations or pharmaceutical formulations of the disclosure may be delivered via direct vaporization or via an electronic inhalation device.

In some embodiments, the GRAS formulation of the disclosure is an edible formulation comprising one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing). “Edible formulation” may refer to a composition suitable for consumption, typically via the oral cavity (although consumption may occur via non-oral means such as inhalation). Edible formulations may be present in any form including, but not limited to, liquids, solids, semi-solids, tablets, lozenges, powders, gels, gums, pastes, slurries, syrups, aerosols, and sprays. As used herein, edible formulations may include food products, pharmaceutical formulations, and consumer products. Edible formulations may also refer to, for example, dietary and nutritional supplements. As used herein, edible formulations may also include formulations that are placed within the oral cavity but not swallowed.

“Food product” may refer to any formulations comprising one or more processed foodstuff. Food products include, but are not limited to, confectionaries, bakery products, ice creams, dairy products, cheeses, sweet and savory snacks, snack foods, beverages (including, but not limited to, hot and cold beverages, beverage mixes, concentrates, juices, carbonated beverages, non-carbonated beverages, alcoholic beverages, non-alcoholic beverages, soft drinks, sports drinks, isotonic drinks, coffees, teas, bottled waters, and beverages prepared from botanicals and botanical extracts), snack bars, meal replacement products, ready meals (including, but not limited to canned meals, preserved meals, frozen meals, dried meals, chilled meals, dinner mixes, and prepared salads), soups, broth, prepared foods (including, but not limited to, dried, canned, or jarred sauces and soups), canned foods, frozen foods, dried foods, chilled foods, oils and fats, sauces, jellies, jams, preserves, honey, puddings, recipe mixes, syrups, icings, fillings, infused foods, and condiments. In some embodiments, the food product is animal feed. For example, the food product may be a pet food product, i.e. a food product for consumption by a household pet. In other embodiments, the food product is a livestock food product, i.e. a food product for consumption by livestock.

“Foodstuff” may refer to an unprocessed ingredient or a basic nutrient or flavor containing element used to prepare a food product. Non-limiting examples of foodstuffs include: fruits, vegetables, meats, fishes, grains, milks, eggs, tubers, sugars, sweeteners, oils, herbs, snacks, sauces, spices and salts.

In some embodiments, the formulation of the disclosure comprising one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) may be a consumer product. “Consumer products” may refer to health, beauty, and general wellness products for the personal use and/or consumption by a subject. Consumer products may be present in any form including, but not limited to, liquids, solids, semi-solids, tablets, capsules, lozenges, strips, powders, gels, gums, pastes, slurries, syrups, aerosols and sprays. Non-limiting examples of consumer products include nutraceuticals, nutritional supplements, cosmetics, sunscreens, lotions, creams, wipes, lipsticks, lip balms, soaps, shampoos, gums, dissolvable films, adhesives (e.g., dental adhesives), toothpastes, breath fresheners, mouthwashes, and other dentifrices.

### Methods of Treatment and Uses of Cannabinoid and Cannabinoid Derivative Preparations and Formulations

The cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) and the pharmaceutical formulations of the disclosure may be useful as pharmaceuticals, as discussed herein.

In some embodiments, one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) are useful as pharmaceuticals.

In some embodiments, the pharmaceutical formulation useful as a pharmaceutical comprises one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing).

The present disclosure provides a method for treating a disorder in a subject including the step of administering to a subject in need thereof a therapeutically effective amount of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing).

The present disclosure provides a method for treating a disorder in a subject including the step of administering to a subject in need thereof a therapeutically effective amount of one or more pharmaceutical formulations of the present disclosure. In some embodiments, the present disclosure provides a method for treating a disorder in a subject including the step of administering to a subject in need thereof a therapeutically effective amount of one or more pharmaceutical formulations comprising one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing).

A “subject” may encompass both mammals and non-mammals. Examples of mammals may include, but are not limited to, any member of the class Mammalia: humans; non-human primates such as chimpanzees, monkeys, baboons, or rhesus monkeys, as well as other apes and monkey species; livestock or farm animals such as cattle, horses, sheep, goats, and swine; household pets or companion animals such as rabbits, dogs, and cats; laboratory animals including rodents, such as rats, mice and guinea pigs; and the like. Examples of non-mammals include, but are not limited to, birds, fish, and the like. “Subject” may include both humans and animals. In some embodiments, the subject is a human.

The terms “effective amount” or “therapeutically effective amount” when used in connection with one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) or pharmaceutical formulations disclosed herein may refer to a sufficient amount of the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) or pharmaceutical formulations disclosed herein to provide the desired biological result. That result can be reduction and/or alleviation of the signs, symptoms, or causes of a disorder, or any other desired alteration of a biological system. For example, a “therapeutically effective amount” or “effective amount” for therapeutic use may be the amount of the pharmaceutical formulation comprising one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) required to provide a clinically significant decrease in a disorder. An appropriate “therapeutically effective amount” or “effective amount” in any individual case may be determined by one of ordinary skill in the art using routine experimentation.

As used herein, the terms “treat” or “treatment” may be meant to indicate a postponement of development of disorders; and/or reducing severity of such symptoms that will or are expected to develop. Thus, these terms may include ameliorating existing disorder symptoms; preventing additional symptoms; ameliorating or preventing the underlying causes of symptoms; inhibiting the disorder, e.g., arresting the development of the disorder; relieving the disorder; causing regression of the disorder; relieving a symptom caused by the disorder; or stopping or alleviating the symptoms of the disorder.

The terms “administered,” “administration,” or “administering” as used in this disclosure may refer to either directly administering one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) or formulations of the disclosure to a subject.

The present disclosure provides for use of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) in the manufacture of a medicament for treating a disorder in a subject in need thereof.

The present disclosure provides for use of one or more pharmaceutical formulations of the present disclosure in the manufacture of a medicament for treating a disorder in a subject in need thereof. In some embodiments, the present disclosure provides for use of one or more pharmaceutical formulations comprising one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) in the manufacture of a medicament for treating a disorder in a subject in need thereof.

The present disclosure provides for use of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) for treating a disorder in a subject in need thereof.

The present disclosure provides for use of one or more pharmaceutical formulations of the present disclosure for treating a disorder in a subject in need thereof. In some embodiments, the present disclosure provides for use of one or more pharmaceutical formulations comprising one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) for treating a disorder in a subject in need thereof.

The present disclosure provides one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), for use in a method of treating a disorder in a subject in need thereof.

The present disclosure provides one or more pharmaceutical formulations of the present disclosure for use in a method of treating a disorder in a subject in need thereof. In some embodiments, the present disclosure provides one or more pharmaceutical formulations comprising one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or a pharmaceutically acceptable salt of any of the foregoing) for use in a method of treating a disorder in a subject in need thereof.

In some embodiments, the disorder of the methods, uses, preparations for use, medicaments, or GRAS formulations or pharmaceutical formulations for use of the disclosure is mediated by cannabinoid receptor type 1 or cannabinoid receptor type 2. In some embodiments, the disorder is mediated by cannabinoid receptor type 1. In some embodiments, the disorder is mediated by cannabinoid receptor type 2.

In some embodiments, the disorder of the methods, uses, preparations for use, medicaments, or GRAS formulations or pharmaceutical formulations for use of the disclosure is chronic pain, multiple sclerosis, cancer-associated nausea and vomiting, weight loss, appetite loss, spasticity, or seizures. In some embodiments, the disorder is chronic pain. In some embodiments, the disorder is multiple sclerosis. In some embodiments, the disorder is cancer-associated nausea and vomiting. In some embodiments, the disorder is weight loss. In some embodiments, the disorder is appetite loss. In some embodiments, the disorder is spasticity. In some embodiments, the disorder is seizures.

In some embodiments, the disorder of the methods, uses, preparations for use, medicaments, or GRAS formulations or pharmaceutical formulations for use of the disclosure is arthritis, depression, rheumatoid arthritis, osteoarthritis, nausea, alcohol use disorders, dermatitis, eczema, acne vulgaris, asthma, angina, cardiovascular disease, autoimmune disease, immunodeficiency disease, an inflammatory disorder, gastritis, epilepsy, encephalopathy, neuropathic pain, cancer (e.g., colorectal), psoriasis, Huntington's disease, anorexia, bladder dysfunction, anxiety, opioid use disorder, dry skin syndrome, autism, Parkinson's disease, obesity, diabetes, or inflammatory bowel disease.

For the therapeutic methods, uses, preparations for use, medicaments, and GRAS formulations or pharmaceutical formulations for use mentioned herein, the dosage administered will, of course, vary with the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) or GRAS formulations or pharmaceutical formulations of the disclosure employed, the mode of administration, the treatment desired and the disorder indicated.

It will be understood, however, that the total daily usage of the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) or GRAS formulations or pharmaceutical formulations of the present disclosure may be decided by the attending physician or veterinarian within the scope of sound medical judgment. The specific therapeutically effective dose level for any particular subject may depend upon a variety of factors including the disorder being treated and the severity of the disorder; the activity of the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) or GRAS formulations or pharmaceutical formulations of the present disclosure employed; the specific GRAS or pharmaceutical formulation employed; the age, body weight, general health, sex and diet of the subject; the time of administration, route of administration, and rate of excretion of the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) or GRAS formulations or pharmaceutical formulations of the present disclosure employed; the duration of the treatment; drugs used in combination or coincidental with the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), or GRAS formulations or pharmaceutical formulations of the present disclosure employed; and like factors well known in the medical arts. A physician or veterinarian of ordinary skill in the art can readily determine and prescribe the therapeutically effective amount of the one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing) or GRAS formulations or pharmaceutical formulations disclosed herein required to treat, counter, or arrest the progress of the disorder.

In some embodiments, one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), or formulations (including GRAS and/or pharmaceutical formulations) described herein, are useful as a nutraceutical. In some embodiments, the nutraceutical is useful for sport or athletic recovery. As used herein, “sport or athletic recovery” may include, but is not limited to, muscle repair and recovery and alleviation or treatment of soreness, swelling, pain, and inflammation associated with participation in sports, athletics, training, and/or exercise.

The present disclosure provides for use of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), or formulations (including GRAS and/or pharmaceutical formulations) described herein, in the manufacture of a nutraceutical. In some embodiments, the nutraceutical is useful for sport or athletic recovery.

The present disclosure provides for use of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), or formulations (including GRAS and/or pharmaceutical formulations) described herein, as a nutraceutical. In some embodiments, the nutraceutical is useful for sport or athletic recovery.

The present disclosure provides for use of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), or formulations (including GRAS and/or pharmaceutical formulations) described herein, in the manufacture of an edible formulation. In certain such embodiments, the edible formulation is a food product or a consumer product. In some embodiments, the food product or consumer product is useful for sport or athletic recovery.

The present disclosure provides for use of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), or formulations (including GRAS and/or pharmaceutical formulations) described herein, in an edible formulation. In certain such embodiments, the edible formulation is a food product or consumer product. In some embodiments, the food product or a consumer product is useful for sport or athletic recovery.

The present disclosure provides for use of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), or formulations (including GRAS and/or pharmaceutical formulations) described herein, in the manufacture of a consumer product. In some embodiments, the consumer product is useful for sport or athletic recovery.

The present disclosure provides for use of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), or formulations (including GRAS and/or pharmaceutical formulations) described herein, in a consumer product. In some embodiments, the consumer product is useful for sport or athletic recovery.

The present disclosure provides for use of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), or formulations (including GRAS and/or pharmaceutical formulations) described herein, in the manufacture of a food product. In some embodiments, the food product is useful for sport or athletic recovery.

The present disclosure provides for use of one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), or formulations (including GRAS and/or pharmaceutical formulations) described herein, in a food product. In some embodiments, the food product is useful for sport or athletic recovery.

In some embodiments, the methods, uses, preparations for use, medicaments, or GRAS formulations or pharmaceutical formulations for use of the disclosure comprise one or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing). In some embodiments, the methods, uses, preparations for use, medicaments, or GRAS formulations or pharmaceutical formulations for use of the disclosure comprise a cannabinoid or cannabinoid derivative preparation disclosed herein (e.g., a preparation comprising an acidic cannabinoid, an acidic cannabinoid derivative, a neutral cannabinoid, or a neutral cannabinoid derivative, or an acceptable or pharmaceutically acceptable salt of any of the foregoing). In some embodiments, the methods, uses, preparations for use, medicaments, or GRAS formulations or pharmaceutical formulations for use of the disclosure comprise two or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing). In some embodiments, the methods, uses, preparations for use, medicaments, or GRAS formulations or pharmaceutical formulations for use of the disclosure comprise three or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing). In some embodiments, the methods, uses, preparations for use, medicaments, or GRAS formulations or pharmaceutical formulations for use of the disclosure comprise four or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing). In some embodiments, the methods, uses, preparations for use, medicaments, or GRAS formulations or pharmaceutical formulations for use of the disclosure comprise five or more cannabinoid or cannabinoid derivative preparations disclosed herein (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing).

### Combination Therapy

In some embodiments, one or more cannabinoid or cannabinoid derivative preparations (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), GRAS formulations, or pharmaceutical formulations described herein may be used alone or together or conjointly administered, or used in combination, with a known therapeutic agent, GRAS formulation, or pharmaceutical formulation. Conjoint administration or used in combination may refer to any form of administration of two or more different therapeutic agents, preparations, GRAS formulations, or pharmaceutical formulations such that the second therapeutic agent, preparation, GRAS formulation, or pharmaceutical formulation is administered while the previously administered preparation, GRAS formulation, or pharmaceutical formulation is still active in the body. For example, the different therapeutic agents, preparations, GRAS formulations, or pharmaceutical formulations can be administered either in the same formulation or in a separate formulation, either simultaneously, sequentially, or by separate dosing of the individual components of the treatment. In some embodiments, the different therapeutic agents, preparations, GRAS formulations, or pharmaceutical formulations can be administered within about one hour, about 12 hours, about 24 hours, about 36 hours, about 48 hours, about 72 hours, or about a week of one another. Thus, a subject who receives such treatment can benefit from a combined effect of different therapeutic agents, preparations, GRAS formulations, or pharmaceutical formulations.

In some embodiments, one or more cannabinoid or cannabinoid derivative preparations (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), GRAS formulations, or pharmaceutical formulations of the disclosure are used in combination with one or more other cannabinoid or cannabinoid derivative preparations (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), GRAS formulations, or pharmaceutical formulations of the disclosure in the methods, uses, preparations for use, medicaments, or GRAS formulations or pharmaceutical formulations for use of the disclosure. In certain such embodiments, the combination of one or more other cannabinoid or cannabinoid derivative preparations (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), GRAS formulations, or pharmaceutical formulations of the disclosure is used in a method for treating one or more of the disorders listed herein.

In some embodiments, combinations of one or more cannabinoid or cannabinoid derivative preparations (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), GRAS formulations, or pharmaceutical formulations provided herein, or combinations of other known agents, GRAS formulations, or pharmaceutical formulations and one or more cannabinoid or cannabinoid derivative preparations (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), GRAS formulations, or pharmaceutical formulations provided herein, are formulated into GRAS formulations, pharmaceutical formulations, and medicaments that are useful in the methods, uses, preparations for use, medicaments, or GRAS formulations or pharmaceutical formulations for use of the disclosure. The disclosure also provides for use of such combinations in treating one or more of the disorders listed herein.

In some embodiments of the disclosure, one or more cannabinoid or cannabinoid derivative preparations (e.g., preparations comprising an acidic cannabinoid, an acidic cannabinoid derivative, a neutral cannabinoid, or a neutral cannabinoid derivative, or an acceptable or pharmaceutically acceptable salt of any of the foregoing), GRAS formulations, or pharmaceutical formulations of the disclosure are administered at a sub-therapeutic dose, wherein a subtherapeutic dose is a dose that would be insufficient to treat one of the disorders listed herein if administered alone.

### Examples of Non-Limiting Embodiments of the Disclosure

Embodiments of the present subject matter disclosed herein may be beneficial alone or in combination with one or more other embodiments. Without limiting the foregoing description, certain non-limiting embodiments of the disclosure, numbered 1 to 65, are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below.

Embodiment 1. A cannabinoid or cannabinoid derivative preparation prepared from a fermentation broth, the cannabinoid or cannabinoid derivative preparation comprising at least 85 weight % of a cannabinoid or cannabinoid derivative.

Embodiment 2. The cannabinoid or cannabinoid derivative preparation of embodiment 1, the cannabinoid or cannabinoid derivative preparation comprising at least 90 weight % of a cannabinoid or cannabinoid derivative.

Embodiment 3. The cannabinoid or cannabinoid derivative preparation of embodiment 1, the cannabinoid or cannabinoid derivative preparation comprising at least 95 weight % of a cannabinoid or cannabinoid derivative.

Embodiment 4. The cannabinoid or cannabinoid derivative preparation of any one of embodiments 1-3 substantially free of impurities.

Embodiment 5. The cannabinoid or cannabinoid derivative preparation of embodiment 4, wherein the cannabinoid or cannabinoid derivative preparation is substantially free of one or more of pentyldiacetic acid lactone (PDAL), hexanoyl triacetic acid lactone (HTAL), olivetol, olivetolic acid, and hexanoic acid.

Embodiment 6. The cannabinoid or cannabinoid derivative preparation of embodiment 4, wherein the cannabinoid or cannabinoid derivative preparation contains less than 5 weight % total of the combination of one or more of pentyldiacetic acid lactone (PDAL), hexanoyl triacetic acid lactone (HTAL), olivetol, olivetolic acid, and hexanoic acid.

Embodiment 7. The cannabinoid or cannabinoid derivative preparation of any one of embodiments 1-6, wherein the cannabinoid is a neutral cannabinoid, wherein the neutral cannabinoid is tetrahydrocannabinol (THC), cannabidiol (CBD), or cannabigerol (CBG).

Embodiment 8. The cannabinoid or cannabinoid derivative preparation of any one of embodiments 1-6, wherein the cannabinoid is an acidic cannabinoid, wherein the acidic cannabinoid is tetrahydrocannabinolic acid (THCA), cannabidiolic acid (CBDA), or cannabigerolic acid (CBGA).

Embodiment 9. The cannabinoid or cannabinoid derivative preparation of any one of embodiments 1-8, wherein the cannabinoid or cannabinoid derivative preparation is a white crystalline solid at about 25° C.

Embodiment 10. The cannabinoid or cannabinoid derivative preparation of any one of embodiments 1-9, wherein the fermentation broth comprises yeast cells, a culture medium, or both yeast cells and culture medium.

Embodiment 11. A method of preparing a cannabinoid or cannabinoid derivative preparation, said method comprising the steps of:

1) extracting an acidic cannabinoid or acidic cannabinoid derivative from a fermentation broth into an emollient phase;

2) extracting the acidic cannabinoid or acidic cannabinoid derivative in emollient into an aqueous phase;

3) decarboxylating the acidic cannabinoid or acidic cannabinoid derivative in the aqueous phase to afford a neutral cannabinoid or neutral cannabinoid derivative;

4) crystallizing the neutral cannabinoid or neutral cannabinoid derivative; and

5) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 12. The method of embodiment 11, said method comprising a step of washing the fermentation broth before extracting the acidic cannabinoid or acidic cannabinoid derivative into the emollient phase.

Embodiment 13. The method of embodiment 11 or 12, said method comprising a step of solubilizing the crystallized neutral cannabinoid or neutral cannabinoid derivative and recrystallizing the neutral cannabinoid or neutral cannabinoid derivative.

Embodiment 14. A method of preparing a cannabinoid or cannabinoid derivative preparation, said method comprising: extracting an acidic cannabinoid or acidic cannabinoid derivative from a fermentation broth using an emollient phase and recovering the resulting cannabinoid or cannabinoid derivative preparation, wherein the cannabinoid or cannabinoid derivative preparation comprises at least 85 weight % of a cannabinoid or cannabinoid derivative, wherein the cannabinoid or cannabinoid derivative is a neutral cannabinoid, a neutral cannabinoid derivative, the acidic cannabinoid, or the acidic cannabinoid derivative.

Embodiment 15. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative in the aqueous phase is subjected to chromatography (e.g. HPLC); and/or 6) the cannabinoid or cannabinoid derivative is precipitated from an aqueous layer.

Embodiment 16. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative in the aqueous phase is subjected to chromatography (e.g. HPLC); 6) the cannabinoid or cannabinoid derivative is precipitated from an aqueous layer; and/or 7) the cannabinoid or cannabinoid derivative is crystallized.

Embodiment 17. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative in the aqueous phase is subjected to chromatography (e.g. HPLC); and/or 6) the cannabinoid or cannabinoid derivative is crystallized.

Embodiment 18. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative is precipitated from an aqueous layer; and/or 6) the cannabinoid or cannabinoid derivative is crystallized.

Embodiment 19. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; and/or 6) the cannabinoid or cannabinoid derivative is precipitated from an aqueous layer.

Embodiment 20. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; and/or 5) the cannabinoid or cannabinoid derivative is crystallized.

Embodiment 21. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative in the aqueous phase is subjected to chromatography (e.g. HPLC); and/or 7) the cannabinoid or cannabinoid derivative is precipitated from an aqueous layer.

Embodiment 22. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative in the aqueous phase is subjected to chromatography (e.g. HPLC); 7) the cannabinoid or cannabinoid derivative is precipitated from an aqueous layer; and/or 8) the cannabinoid or cannabinoid derivative is crystallized.

Embodiment 23. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative in the aqueous phase is subjected to chromatography (e.g. HPLC); and/or 7) the cannabinoid or cannabinoid derivative is crystallized.

Embodiment 24. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative in the aqueous phase is subjected to chromatography (e.g. HPLC); and/or 5) the cannabinoid or cannabinoid derivative is precipitated from an aqueous layer.

Embodiment 25. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative in the aqueous phase is subjected to chromatography (e.g. HPLC); 5) the cannabinoid or cannabinoid derivative is precipitated from an aqueous layer; and/or 6) the cannabinoid or cannabinoid derivative is crystallized.

Embodiment 26. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative in the aqueous phase is subjected to chromatography (e.g. HPLC); and/or 5) the cannabinoid or cannabinoid derivative is crystallized.

Embodiment 27. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative is precipitated from an aqueous layer; and/or 5) the cannabinoid or cannabinoid derivative is crystallized.

Embodiment 28. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; and/or 4) the cannabinoid or cannabinoid derivative is precipitated from an aqueous layer.

Embodiment 29. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; and/or 4) the cannabinoid or cannabinoid derivative is crystallized.

Embodiment 30. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 5) the cannabinoid or cannabinoid derivative in the aqueous phase is subjected to chromatography (e.g. HPLC); and/or 6) the cannabinoid or cannabinoid derivative is precipitated from an aqueous layer.

Embodiment 31. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 5) the cannabinoid or cannabinoid derivative in the aqueous phase is subjected to chromatography (e.g. HPLC); 6) the cannabinoid or cannabinoid derivative is precipitated from an aqueous layer; and/or 7) the cannabinoid or cannabinoid derivative is crystallized.

Embodiment 32. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 5) the cannabinoid or cannabinoid derivative in the aqueous phase is subjected to chromatography (e.g. HPLC); and/or 6) the cannabinoid or cannabinoid derivative is crystallized.

Embodiment 33. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative in the aqueous phase is subjected to chromatography (e.g. HPLC); and/or 6) the cannabinoid or cannabinoid derivative is precipitated from an aqueous layer.

Embodiment 34. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative in the aqueous phase is subjected to chromatography (e.g. HPLC); 6) the cannabinoid or cannabinoid derivative is precipitated from an aqueous layer; and/or 7) the cannabinoid or cannabinoid derivative is crystallized; and 8) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 35. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative in the aqueous phase is subjected to chromatography (e.g. HPLC); and/or 6) the cannabinoid or cannabinoid derivative is crystallized; and 7) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 36. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) the cannabinoid or cannabinoid derivative is precipitated from an aqueous layer; and/or 6) the cannabinoid or cannabinoid derivative is crystallized; and 7) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 37. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; and/or 5) the cannabinoid or cannabinoid derivative is precipitated from an aqueous layer; and 6) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 38. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; and/or 5) the cannabinoid or cannabinoid derivative is crystallized; and 6) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 39. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative in the aqueous phase is subjected to chromatography (e.g. HPLC); and/or 7) the cannabinoid or cannabinoid derivative is precipitated from an aqueous layer; and 8) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 40. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative in the aqueous phase is subjected to chromatography (e.g. HPLC); 7) the cannabinoid or cannabinoid derivative is precipitated from an aqueous layer; and/or 8) the cannabinoid or cannabinoid derivative is crystallized; and 9) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 41. A method of preparing a cannabinoid or cannabinoid derivative preparation, said method comprising: 1) modified host cells in fermentation broth are washed to solubilize and separate impurities from the fermentation broth; 2) the washed fermentation broth is centrifuged to separate the biomass from the fermentation broth; 3) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 4) the cannabinoid or cannabinoid derivative is extracted into a second aqueous phase; 5) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 6) the cannabinoid or cannabinoid derivative in the aqueous phase is subjected to chromatography (e.g. HPLC); and/or 7) the cannabinoid or cannabinoid derivative is crystallized; and 8) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 42. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative in the aqueous phase is subjected to chromatography (e.g. HPLC); and/or 5) the cannabinoid or cannabinoid derivative is precipitated from an aqueous layer; and 6) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 43. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative in the aqueous phase is subjected to chromatography (e.g. HPLC); 5) the cannabinoid or cannabinoid derivative is precipitated from an aqueous layer; and/or 6) the cannabinoid or cannabinoid derivative is crystallized; and 7) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 44. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative in the aqueous phase is subjected to chromatography (e.g. HPLC); and/or 5) the cannabinoid or cannabinoid derivative is crystallized; and 6) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 45. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) the cannabinoid or cannabinoid derivative is precipitated from an aqueous layer; and/or 5) the cannabinoid or cannabinoid derivative is crystallized; and 6) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 46. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; and/or 4) the cannabinoid or cannabinoid derivative is precipitated from an aqueous layer; and 5) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 47. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; and/or 4) the cannabinoid or cannabinoid derivative is crystallized; and 5) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 48. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 5) the cannabinoid or cannabinoid derivative in the aqueous phase is subjected to chromatography (e.g. HPLC); and/or 6) the cannabinoid or cannabinoid derivative is precipitated from an aqueous layer; and 7) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 49. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 5) the cannabinoid or cannabinoid derivative in the aqueous phase is subjected to chromatography (e.g. HPLC); 6) the cannabinoid or cannabinoid derivative is precipitated from an aqueous layer; and/or 7) the cannabinoid or cannabinoid derivative is crystallized; and 8) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 50. A method of preparing a cannabinoid or cannabinoid derivative preparation, said method comprising: 1) the fermentation broth containing the modified host cells of the disclosure is centrifuged to separate the biomass from the fermentation broth; 2) a cannabinoid or cannabinoid derivative (e.g., an acidic cannabinoid, acidic cannabinoid derivative, neutral cannabinoid, or neutral cannabinoid derivative) produced by the modified host cells is extracted into an emollient phase; 3) the cannabinoid or cannabinoid derivative is extracted into a second aqueous phase; 4) if the cannabinoid or cannabinoid derivative is an acidic cannabinoid or acidic cannabinoid derivative, the acidic cannabinoid or acidic cannabinoid derivative may be decarboxylated to afford a neutral cannabinoid or neutral cannabinoid derivative (this step can be omitted if the acidic cannabinoid or acidic cannabinoid derivative is the desired product); 5) the cannabinoid or cannabinoid derivative in the aqueous phase is subjected to chromatography (e.g. HPLC); and/or 6) the cannabinoid or cannabinoid derivative is crystallized; and 7) recovering the resulting cannabinoid or cannabinoid derivative preparation, wherein the cannabinoid or cannabinoid derivative preparation comprises at least 85 weight % of the neutral cannabinoid or neutral cannabinoid derivative.

Embodiment 51. The method of any of Embodiments 11-50, the steps include a measurement of purification and/or yield before any one step.

Embodiment 52. The method of any of Embodiments 11-51, wherein the steps include a measurement of purification and/or yield before one or more steps.

Embodiment 53. The method of any of Embodiments 11-52, wherein the steps include a measurement of purification and/or yield before each step.

Embodiment 54. The method of any one of embodiments 11-53, wherein the pH of the fermentation broth is between about 4.0 and about 10.0.

Embodiment 55. The method of embodiment 54, wherein the pH of the fermentation broth is between about 5.0 and about 9.0.

Embodiment 56. The method of embodiment 55, wherein the pH of the fermentation broth is between about 7.0 and about 8.0.

Embodiment 57. The method of embodiment 56, wherein the pH of the fermentation broth is about 7.67.

Embodiment 58. The method of any one of embodiments 11-57, wherein the extraction of the acidic cannabinoid or acidic cannabinoid derivative from the fermentation broth into the emollient phase is performed at a temperature between about 20° C. and about 50° C.

Embodiment 59. The method of any one of embodiments 11-57, wherein the extraction of the acidic cannabinoid or acidic cannabinoid derivative from the fermentation broth into the emollient phase is performed at a temperature between about 30° C. and about 50° C.

Embodiment 60. The method of embodiment 58, wherein the extraction of the acidic cannabinoid or acidic cannabinoid derivative from the fermentation broth into the emollient phase is performed at a temperature of about 30° C.

Embodiment 61. The method of any one of embodiments 11-20, wherein the extraction of the acidic cannabinoid or acidic cannabinoid derivative from the fermentation broth into the emollient phase is performed using about 10% v/v to about 20% v/v emollient.

Embodiment 62. The method of any one of embodiments 11-61, wherein at least about 50% of the acidic cannabinoid or acidic cannabinoid derivative present in the fermentation broth is extracted into the emollient phase.

Embodiment 63. The method of embodiment 62, wherein at least about 90% of the acidic cannabinoid or acidic cannabinoid derivative present in the fermentation broth is extracted into the emollient phase.

Embodiment 64. The method of embodiment 63, wherein at least about 95% of the acidic cannabinoid or acidic cannabinoid derivative present in the fermentation broth is extracted into the emollient phase.

Embodiment 65. The method of any one of embodiments 11-64, wherein one or more impurities present in the fermentation broth are not significantly extracted into the emollient phase with the acidic cannabinoid or acidic cannabinoid derivative.

Embodiment 66. The method of embodiments 65, wherein the one or more impurities comprise olivetolic acid, PDAL, or HTAL, or a combination of any of the foregoing.

Embodiment 67. The method of any one of embodiments 14-66, wherein the method comprises a step of extracting the acidic cannabinoid or acidic cannabinoid derivative in an emollient phase into an aqueous phase.

Embodiment 68. The method of any one of embodiments 11-67, wherein the extraction of the acidic cannabinoid or acidic cannabinoid derivative in the emollient phase into the aqueous phase is performed at a ratio of water:emollient phase between about 1:1 and about 5:1.

Embodiment 69. The method of embodiment 68, wherein the extraction of the acidic cannabinoid or acidic cannabinoid derivative in the emollient phase into the aqueous phase is performed at a ratio of water: emollient phase of about 4:1

Embodiment 70. The method of embodiment 68 or 71, wherein the acidic cannabinoid or acidic cannabinoid derivative in the emollient phase is extracted into the aqueous phase at a pH of about 11.5.

Embodiment 71. The method of any one of embodiments 11-70, wherein the extraction of the acidic cannabinoid or acidic cannabinoid derivative in the emollient phase into the aqueous phase is performed by:

a) mild agitation; or

b) vigorous mixing to hydrolyze the emollient phase.

Embodiment 72. The method of any one of embodiments 11-71, wherein at least about 50% of the acidic cannabinoid or acidic cannabinoid derivative present in the emollient phase is extracted into the aqueous phase.

Embodiment 73. The method of embodiment 72, wherein at least 90% of the acidic cannabinoid or acidic cannabinoid derivative present in the emollient phase is extracted into the aqueous phase.

Embodiment 74. The method of any of embodiments 11-73, wherein the emollient phase comprises an oil, solvent, toluene, methyl isobutyl ketone (MIBK), heptanes, ethanol, methanol, isopropanol, isopropyl myristate (IPM), or any combination thereof.

Embodiment 75. The method of embodiment 74, wherein the emollient phase comprises IPM.

Embodiment 76. The method of any one of embodiments 14-75, wherein the cannabinoid or cannabinoid derivative is the neutral cannabinoid or neutral cannabinoid derivative.

Embodiment 77. The method of embodiment 76, wherein the method comprises a step of decarboxylating the acidic cannabinoid or acidic cannabinoid derivative to afford the neutral cannabinoid or neutral cannabinoid derivative.

Embodiment 78. The method of any one of embodiments 11-27, wherein decarboxylation of the acidic cannabinoid or acidic cannabinoid derivative is performed at temperatures greater than about 70° C.

Embodiment 79. The method of embodiment 78, wherein decarboxylation of the acidic cannabinoid or acidic cannabinoid derivative is performed at a temperature between about 80° C. to about 140° C.

Embodiment 80. The method of embodiment 79, wherein decarboxylation of the acidic cannabinoid or acidic cannabinoid derivative is performed at a temperature between about 90° C. to about 130° C.

Embodiment 81. The method of any one of embodiments 11-80, wherein decarboxylation of the acidic cannabinoid or acidic cannabinoid derivative is performed for about 5 minutes to about 20 hours.

Embodiment 82. The method of embodiment 81, wherein decarboxylation of the acidic cannabinoid or acidic cannabinoid derivative is performed for about 14 hours.

Embodiment 83. The method of embodiment 81, wherein decarboxylation of the acidic cannabinoid or acidic cannabinoid derivative is performed for about 20 minutes.

Embodiment 84. The method of any one of embodiments 11-83, wherein the yield of the neutral cannabinoid or neutral cannabinoid derivative afforded by decarboxylation of the acidic cannabinoid or acidic cannabinoid derivative is at least about 50%.

Embodiment 85. The method of embodiment 84, wherein the yield of the neutral cannabinoid or neutral cannabinoid derivative afforded by decarboxylation of the acidic cannabinoid or acidic cannabinoid derivative is at least about 60%.

Embodiment 86. The method of embodiment 85, wherein the yield of the neutral cannabinoid or neutral cannabinoid derivative afforded by decarboxylation of the acidic cannabinoid or acidic cannabinoid derivative is between about 60% and about 100%.

Embodiment 87. The method of any one of embodiments 76-86, wherein the method comprises a step of crystallizing the neutral cannabinoid or neutral cannabinoid derivative.

Embodiment 88. The method of any one of embodiments 11-87, wherein crystallization of the neutral cannabinoid or neutral cannabinoid derivative is performed by slowly cooling an aqueous phase comprising the neutral cannabinoid or neutral cannabinoid derivative afforded by decarboxylation of the acidic cannabinoid or acidic cannabinoid derivative to about 25° C. or below.

Embodiment 89. The method of embodiment 88, wherein crystallization of the neutral cannabinoid or neutral cannabinoid derivative occurs at a pH of about 1.0 to about 10.0.

Embodiment 90. The method of embodiment 88, wherein crystallization of the neutral cannabinoid or neutral cannabinoid derivative occurs at a pH of below about 12.

Embodiment 91. The method of embodiment 88, wherein crystallization of the neutral cannabinoid or neutral cannabinoid derivative occurs at a pH of below about 2.

Embodiment 92. The method of any one of embodiments 11-91, wherein the yield of the cannabinoid or cannabinoid derivative preparation recovered after crystallization of the neutral cannabinoid or neutral cannabinoid derivative is at least 50%.

Embodiment 93. The method of embodiment 92, wherein the yield of the cannabinoid or cannabinoid derivative preparation recovered after crystallization of the neutral cannabinoid or neutral cannabinoid derivative is at least 95%.

Embodiment 94. The method of any one of embodiments 11-93, wherein the neutral cannabinoid is tetrahydrocannabinol (THC), cannabidiol (CBD), or cannabigerol (CBG).

Embodiment 95. The method of any one of embodiments 11-94, wherein the cannabinoid or cannabinoid derivative is the acidic cannabinoid or acidic cannabinoid derivative.

Embodiment 96. The method of embodiment 95, wherein the method comprises a step of crystallizing the acidic cannabinoid or acidic cannabinoid derivative.

Embodiment 97. The method of embodiment 95, wherein the yield of the cannabinoid or cannabinoid derivative preparation recovered after crystallization of the acidic cannabinoid or acidic cannabinoid derivative is at least 50%.

Embodiment 98. The method of embodiment 97, wherein the yield of the cannabinoid or cannabinoid derivative is at least 95%.

Embodiment 99. The method of any one of embodiments 95-98, wherein the acidic cannabinoid is tetrahydrocannabinolic acid (THCA), cannabidiolic acid (CBDA), or cannabigerolic acid (CBGA).

Embodiment 100. The method of any one of embodiments 11-99, wherein the fermentation broth comprises yeast cells, a culture medium, or both yeast cells and a culture medium.

Embodiment 101. A cannabinoid or cannabinoid derivative preparation preparable by the method of any one of embodiments 11-100.

Embodiment 102. A cannabinoid or cannabinoid derivative preparation prepared by the method of any one of embodiments 11-100.

Embodiment 103. The cannabinoid or cannabinoid derivative preparation of any one of embodiments 1-10 and 101-102 or the methods of any one of embodiments 11-100, wherein the cannabinoid or cannabinoid derivative is a salt form.

Embodiment 104. The cannabinoid or cannabinoid derivative preparation of any one of embodiments 1-10 and 101-102 or the methods of any one of embodiments 11-100, wherein the cannabinoid or cannabinoid derivative is not a salt form.

### EXAMPLES

### Example 1

### Extraction and Purification of CBG from Recombinant Yeast

Genetically modified yeast are grown in fermentation broth as described in WO2018/200888 filed Apr. 27, 2018 and WO 2020/069214 filed Sep. 26, 2019, and PCT/US2020/051261 filed Sep. 17, 2020, which are herein incorporated by reference in their entirety. In addition to the CBGA, the fermentation broth contains unwanted compounds, such as HTAL, PDAL, olivetol, olivetolic acid, hexanoic acid, and/or CBGVA. The overall process comprises the steps of 1) IPM extraction; 2) extraction into aqueous phase; 3) decarboxylation: and 4) precipitation. Before the IPM extraction, the process may begin with washing of the fermentation broth to solubilize and separate impurities from the fermentation broth. An overview of the process, along with the CBGA/CBG yield at each step is depicted in FIG. 1.

Washing of the Fermentation Broth (Optional)

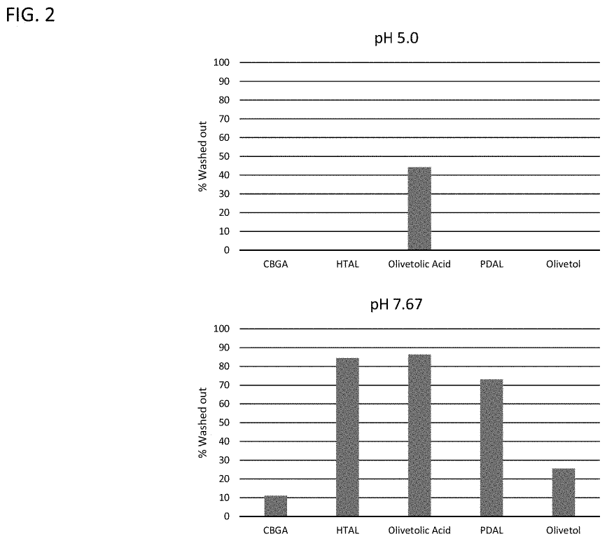

FIG. 2 shows the amounts of cannabigerolic acid (CBGA), hexanoyl triacetic acid lactone (HTAL), olivetolic acid, pentyl diacetic acid lactone (PDAL), and olivetol washed out of the fermentation broth at pH 5.0 and pH 7.67. At pH 7.67, 10% of the CBGA, 85% of the HTAL and olivetolic acid, 75% of PDAL, and 25% olivetol was lost.

Extraction of CBGA from Fermentation Broth into IPM

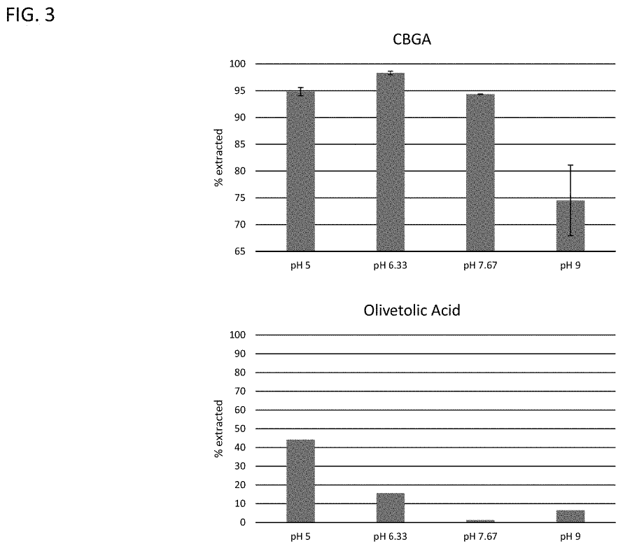

IPM was used to extract CBGA from the fermentation broth. IPM extracts CBGA and olivetol, among other compounds, from the fermentation broth. This extraction leaves behind common impurities and/or byproducts such as olivetolic acid, PDAL, and HTAL. 10% v/v IPM was added to the fermentation broth at the end of fermentation, and the two phases were mixed at 1000 rpm (pH 7.67, 30° C.) for 30 minutes. After mixing, IPM and fermentation broth were run through a three-phase centrifugation, and the insoluble fraction (mostly cells) was separated from the IPM and aqueous phase, and the IPM was separated from the aqueous phase. Approximately 95% of the CBGA from the fermentation broth was extracted into the IPM. (FIG. 3) Importantly, extraction into IPM at 7.67 also resulted in minimal (e.g., below LOQ) extraction for the impurities and/or byproducts olivetolic acid (see FIG. 3), PDAL, and HTAL (data not shown).

Extraction of CBGA in IPM into an Aqueous Phase

The CBGA in IPM obtained through the process described above was extracted from the IPM into a fresh aqueous phase (water). Water was added to the IPM until the solution reached a ratio of water:IPM of 4:1. The pH was brought to 11.5 using 25% KOH. At this pH, the CBGA became soluble and migrated into the aqueous phase. Extraction of the CBGA out of the IPM and into the aqueous phase was performed at mild agitation to avoid emulsification of the IPM and the water. The phases were then separated using a liquid:liquid separator. The liquid:liquid separator takes advantage of the different densities of the two phases: IPM has a density of 850 kg/m³ and the aqueous phase (water) has a density of 997 kg/m³. After the separator mixes the two liquids, it spins them at high speed, and separates them based on gravity.

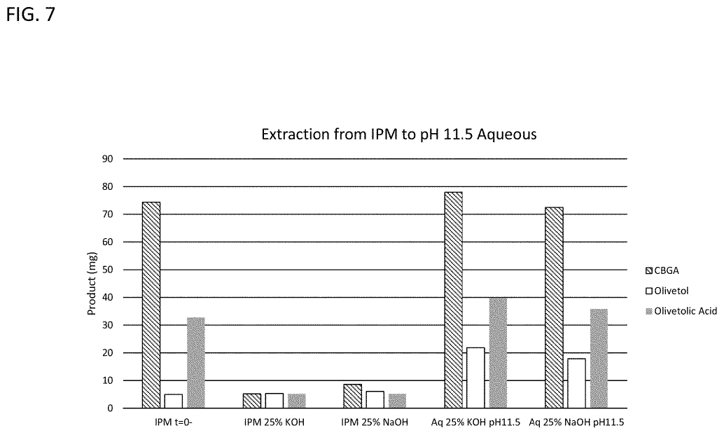

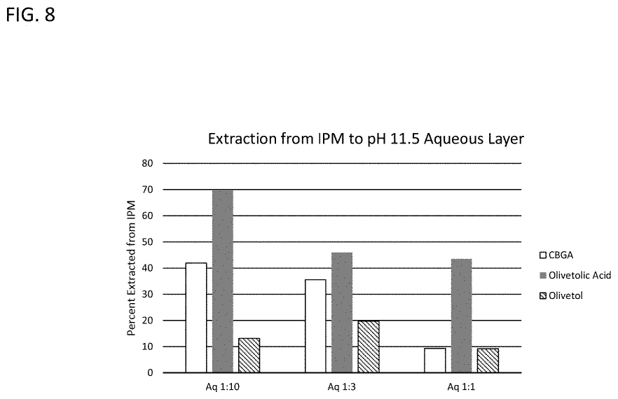

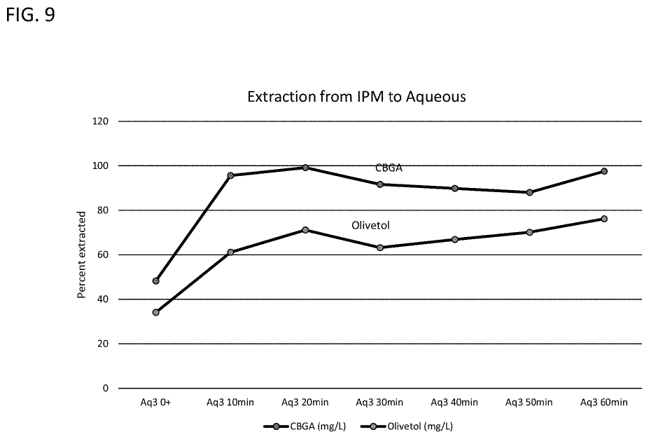

FIGS. 7-9 show the extraction from IPM to pH 11.5 aqueous solution. Aqueous solutions pH adjusted to 11.5 using either 25% KOH or 25% NaOH have similar extraction yield from IPM. However, a fluffy interface layer was seen in the 25% NaOH solution, which is not ideal for clean phase separation.

Decarboxylation

Decarboxylation is a chemical reaction that removes a carboxyl-group from CBGA and releases carbon dioxide to produce CBG. (FIGS. 4-6) The rate of decarboxylation is dependent on heat. Here, the CBGA in the aqueous solution was covered with an N2 overlay to prevent oxidation and unwanted chemical reactions of oxygen with other compounds present in the aqueous phase, and decarboxylation occurred at 90° C. over a period of 14 hours.

Crystallization of CBG

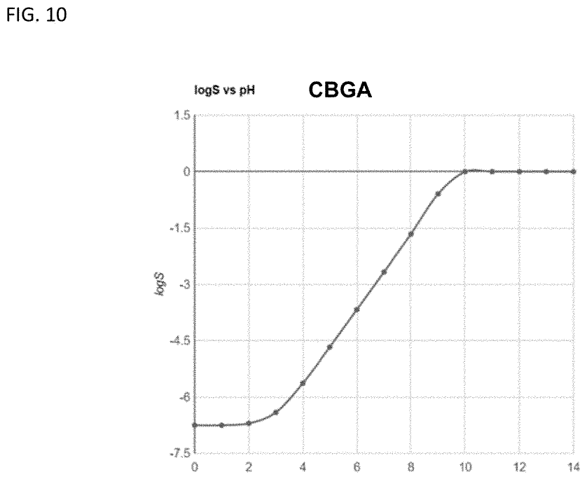

The final step for purifying CGB from a fermentation broth is crystallization of the CBG from the aqueous layer via acidification. Crystallization occurs spontaneously in time when the decarboxylated aqueous phase is cooled slowly to about 25° C. or below. Different molecules fall out of solution at different pHs. For example, CBGA is insoluble in water at pH <2 and CBG is insoluble at pH <10. See FIG. 10. Crystals formed were then recovered using filtration. These can be further purified by washing the crystals using water and/or recrystallization.

In some embodiments, the crystals comprise a substantially pure cannabinoid or cannabinoid derivative. In some embodiments, the purity of the cannabinoid or cannabinoid derivative crystal is determined by chromatographic purity (by area normalization). In some embodiments, the cannabinoid or cannabinoid derivative crystal has a chromatographic purity of about 50% to about 100%, or about 80% to about 99.9%, or about 90% to about 99.9%, or about 95 to about 99.9%. In some embodiments, the cannabinoid or cannabinoid derivative crystal has a chromatographic purity of about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about, 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or about 100% by area normalization.

### Incorporation by Reference

All patents and publications referenced herein are hereby incorporated by reference in their entireties, including the publications disclosed below.

The publications discussed herein are provided solely for their disclosure prior to the filing date of the present application. Nothing herein is to be construed as an admission that the present disclosure is not entitled to antedate such publication by virtue of prior disclosure.

This application incorporates by reference the following publications in their entireties for all purposes: WO2018/200888 filed Apr. 27, 2018; WO 2020/069214 filed Sep. 26, 2019; WO 2020/069142 filed Sep. 26, 2019; WO 2020/236789 filed May 19, 2020; and PCT/US2020/051261 filed Sep. 17, 2020.

1. A cannabinoid or cannabinoid derivative preparation prepared from a fermentation broth, the cannabinoid or cannabinoid derivative preparation comprising at least 85 weight % of a cannabinoid or cannabinoid derivative.

2. The cannabinoid or cannabinoid derivative preparation of claim 1, the cannabinoid or cannabinoid derivative preparation comprising at least 90 weight % of a cannabinoid or cannabinoid derivative.

3. The cannabinoid or cannabinoid derivative preparation of claim 1, the cannabinoid or cannabinoid derivative preparation comprising at least 95 weight % of a cannabinoid or cannabinoid derivative.

4. The cannabinoid or cannabinoid derivative preparation of any one of claims 1-3 substantially free of impurities.

5. The cannabinoid or cannabinoid derivative preparation of claim 4, wherein the cannabinoid or cannabinoid derivative preparation is substantially free of one or more of pentyldiacetic acid lactone (PDAL), hexanoyl triacetic acid lactone (HTAL), olivetol, olivetolic acid, and hexanoic acid.

6. The cannabinoid or cannabinoid derivative preparation of claim 4, wherein the cannabinoid or cannabinoid derivative preparation contains less than 5 weight % total of the combination of one or more of pentyldiacetic acid lactone (PDAL), hexanoyl triacetic acid lactone (HTAL), olivetol, olivetolic acid, and hexanoic acid.

7. The cannabinoid or cannabinoid derivative preparation of any one of claims 1-6, wherein the cannabinoid is a neutral cannabinoid, wherein the neutral cannabinoid is tetrahydrocannabinol (THC), cannabidiol (CBD), or cannabigerol (CBG).

8. The cannabinoid or cannabinoid derivative preparation of any one of claims 1-6, wherein the cannabinoid is an acidic cannabinoid, wherein the acidic cannabinoid is tetrahydrocannabinolic acid (THCA), cannabidiolic acid (CBDA), or cannabigerolic acid (CBGA).

9. The cannabinoid or cannabinoid derivative preparation of any one of claims 1-8, wherein the cannabinoid or cannabinoid derivative preparation is a white crystalline solid at about 25° C.

10. The cannabinoid or cannabinoid derivative preparation of any one of claims 1-9, wherein the fermentation broth comprises yeast cells, a culture medium, or both yeast cells and culture medium.

11. A method of preparing a cannabinoid or cannabinoid derivative preparation, said method comprising the steps of:
1) extracting an acidic cannabinoid or acidic cannabinoid derivative from a fermentation broth into an emollient phase;
2) extracting the acidic cannabinoid or acidic cannabinoid derivative in emollient into an aqueous phase;
3) decarboxylating the acidic cannabinoid or acidic cannabinoid derivative in the aqueous phase to afford a neutral cannabinoid or neutral cannabinoid derivative;
4) crystallizing the neutral cannabinoid or neutral cannabinoid derivative; and
5) recovering the resulting cannabinoid or cannabinoid derivative preparation, wherein the cannabinoid or cannabinoid derivative preparation comprises at least 85 weight % of the neutral cannabinoid or neutral cannabinoid derivative.

12. The method of claim 11, said method comprising a step of washing the fermentation broth before extracting the acidic cannabinoid or acidic cannabinoid derivative into the emollient phase.

13. The method of claim 11 or 12, said method comprising a step of solubilizing the crystallized neutral cannabinoid or neutral cannabinoid derivative and recrystallizing the neutral cannabinoid or neutral cannabinoid derivative.

14. A method of preparing a cannabinoid or cannabinoid derivative preparation, said method comprising: extracting an acidic cannabinoid or acidic cannabinoid derivative from a fermentation broth using an emollient phase and recovering the resulting cannabinoid or cannabinoid derivative preparation, wherein the cannabinoid or cannabinoid derivative preparation comprises at least 85 weight % of a cannabinoid or cannabinoid derivative, wherein the cannabinoid or cannabinoid derivative is a neutral cannabinoid, a neutral cannabinoid derivative, the acidic cannabinoid, or the acidic cannabinoid derivative.

15. The method of any one of claims 11-14, wherein the pH of the fermentation broth is between about 4.0 and about 10.0.

16. The method of claim 15, wherein the pH of the fermentation broth is between about 5.0 and about 9.0.

17. The method of claim 16, wherein the pH of the fermentation broth is between about 7.0 and about 8.0.

18. The method of claim 17, wherein the pH of the fermentation broth is about 7.67.

19. The method of any one of claims 11-18, wherein the extraction of the acidic cannabinoid or acidic cannabinoid derivative from the fermentation broth into the emollient phase is performed at a temperature between about 20° C. and about 50° C.

20. The method of any one of claims 11-18, wherein the extraction of the acidic cannabinoid or acidic cannabinoid derivative from the fermentation broth into the emollient phase is performed at a temperature between about 30° C. and about 50° C.

21. The method of claim 19, wherein the extraction of the acidic cannabinoid or acidic cannabinoid derivative from the fermentation broth into the emollient phase is performed at a temperature of about 30° C.

22. The method of any one of claims 11-21, wherein the extraction of the acidic cannabinoid or acidic cannabinoid derivative from the fermentation broth into the emollient phase is performed using about 10% v/v to about 20% v/v emollient.

23. The method of any one of claims 11-22, wherein at least about 50% of the acidic cannabinoid or acidic cannabinoid derivative present in the fermentation broth is extracted into the emollient phase.

24. The method of claim 23, wherein at least about 90% of the acidic cannabinoid or acidic cannabinoid derivative present in the fermentation broth is extracted into the emollient phase.

25. The method of claim 24, wherein at least about 95% of the acidic cannabinoid or acidic cannabinoid derivative present in the fermentation broth is extracted into the emollient phase.

26. The method of any one of claims 11-25, wherein one or more impurities present in the fermentation broth are not significantly extracted into the emollient phase with the acidic cannabinoid or acidic cannabinoid derivative.

27. The method of claim 26, wherein the one or more impurities comprise olivetolic acid, PDAL, or HTAL, or a combination of any of the foregoing.

28. The method of any one of claims 14-27, wherein the method comprises a step of extracting the acidic cannabinoid or acidic cannabinoid derivative in an emollient phase into an aqueous phase.

29. The method of any one of claims 11-13 and 28, wherein the extraction of the acidic cannabinoid or acidic cannabinoid derivative in the emollient phase into the aqueous phase is performed at a ratio of water:emollient phase between about 1:1 and about 5:1.

30. The method of claim 29, wherein the extraction of the acidic cannabinoid or acidic cannabinoid derivative in the emollient phase into the aqueous phase is performed at a ratio of water:emollient phase of about 4:1.

31. The method of claim 29 or 30, wherein the acidic cannabinoid or acidic cannabinoid derivative in the emollient phase is extracted into the aqueous phase at a pH of about 11.5.

32. The method of any one of claims 11-13 and 28-31, wherein the extraction of the acidic cannabinoid or acidic cannabinoid derivative in the emollient phase into the aqueous phase is performed by:
a) mild agitation; or
b) vigorous mixing to hydrolyze the emollient phase.

33. The method of any one of claims 11-13 and 28-32, wherein at least about 50% of the acidic cannabinoid or acidic cannabinoid derivative present in the emollient phase is extracted into the aqueous phase.

34. The method of claim 33, wherein at least 90% of the acidic cannabinoid or acidic cannabinoid derivative present in the emollient phase is extracted into the aqueous phase.

35. The method of any of claims 11-34, wherein the emollient phase comprises an oil, solvent, toluene, methyl isobutyl ketone (MIBK), heptanes, ethanol, methanol, isopropanol, isopropyl myristate (IPM), or any combination thereof.

36. The method of claim 35, wherein the emollient phase comprises IPM.

37. The method of any one of claims 14-36, wherein the cannabinoid or cannabinoid derivative is the neutral cannabinoid or neutral cannabinoid derivative.

38. The method of claim 37, wherein the method comprises a step of decarboxylating the acidic cannabinoid or acidic cannabinoid derivative to afford the neutral cannabinoid or neutral cannabinoid derivative.

39. The method of any one of claims 11-13 and 38, wherein decarboxylation of the acidic cannabinoid or acidic cannabinoid derivative is performed at temperatures greater than about 70° C.

40. The method of claim 39, wherein decarboxylation of the acidic cannabinoid or acidic cannabinoid derivative is performed at a temperature between about 80° C. to about 140° C.

41. The method of claim 40, wherein decarboxylation of the acidic cannabinoid or acidic cannabinoid derivative is performed at a temperature between about 90° C. to about 130° C.

42. The method of any one of claims 11-13 and 38-41, wherein decarboxylation of the acidic cannabinoid or acidic cannabinoid derivative is performed for about 5 minutes to about 20 hours.

43. The method of claim 42, wherein decarboxylation of the acidic cannabinoid or acidic cannabinoid derivative is performed for about 14 hours.

44. The method of claim 42, wherein decarboxylation of the acidic cannabinoid or acidic cannabinoid derivative is performed for about 20 minutes.

45. The method of any one of claims 11-13 and 38-44, wherein the yield of the neutral cannabinoid or neutral cannabinoid derivative afforded by decarboxylation of the acidic cannabinoid or acidic cannabinoid derivative is at least about 50%.

46. The method of claim 45, wherein the yield of the neutral cannabinoid or neutral cannabinoid derivative afforded by decarboxylation of the acidic cannabinoid or acidic cannabinoid derivative is at least about 60%.

47. The method of claim 46, wherein the yield of the neutral cannabinoid or neutral cannabinoid derivative afforded by decarboxylation of the acidic cannabinoid or acidic cannabinoid derivative is between about 60% and about 100%.

48. The method of any one of claims 37-47, wherein the method comprises a step of crystallizing the neutral cannabinoid or neutral cannabinoid derivative.

49. The method of any one of claims 11-13 and 48, wherein crystallization of the neutral cannabinoid or neutral cannabinoid derivative is performed by slowly cooling an aqueous phase comprising the neutral cannabinoid or neutral cannabinoid derivative afforded by decarboxylation of the acidic cannabinoid or acidic cannabinoid derivative to about 25° C. or below.

50. The method of claim 49, wherein crystallization of the neutral cannabinoid or neutral cannabinoid derivative occurs at a pH of about 1.0 to about 10.0.

51. The method of claim 49, wherein crystallization of the neutral cannabinoid or neutral cannabinoid derivative occurs at a pH of below about 12.

52. The method of claim 49, wherein crystallization of the neutral cannabinoid or neutral cannabinoid derivative occurs at a pH of below about 2.

53. The method of any one of claims 11-13 and 48-52, wherein the yield of the cannabinoid or cannabinoid derivative preparation recovered after crystallization of the neutral cannabinoid or neutral cannabinoid derivative is at least 50%.

54. The method of claim 53, wherein the yield of the cannabinoid or cannabinoid derivative preparation recovered after crystallization of the neutral cannabinoid or neutral cannabinoid derivative is at least 95%.

55. The method of any one of claims 11-13 and 37-54, wherein the neutral cannabinoid is tetrahydrocannabinol (THC), cannabidiol (CBD), or cannabigerol (CBG).

56. The method of any one of claims 14-36, wherein the cannabinoid or cannabinoid derivative is the acidic cannabinoid or acidic cannabinoid derivative.

57. The method of claim 56, wherein the method comprises a step of crystallizing the acidic cannabinoid or acidic cannabinoid derivative.

58. The method of claim 56, wherein the yield of the cannabinoid or cannabinoid derivative preparation recovered after crystallization of the acidic cannabinoid or acidic cannabinoid derivative is at least 50%.

59. The method of claim 58, wherein the yield of the cannabinoid or cannabinoid derivative is at least 95%.

60. The method of any one of claims 56-59, wherein the acidic cannabinoid is tetrahydrocannabinolic acid (THCA), cannabidiolic acid (CBDA), or cannabigerolic acid (CBGA).

61. The method of any one of claims 11-60, wherein the fermentation broth comprises yeast cells, a culture medium, or both yeast cells and a culture medium.

62. A cannabinoid or cannabinoid derivative preparation preparable by the method of any one of claims 11-61.

63. A cannabinoid or cannabinoid derivative preparation prepared by the method of any one of claims 11-61.

64. The cannabinoid or cannabinoid derivative preparation of any one of claims 1-10 and 62-63 or the methods of any one of claims 11-61, wherein the cannabinoid or cannabinoid derivative is a salt form.

65. The cannabinoid or cannabinoid derivative preparation of any one of claims 1-10 and 62-63 or the methods of any one of claims 11-61, wherein the cannabinoid or cannabinoid derivative is not a salt form.
